(19) United States
(12) Patent Application Publication
(10) Pub. No.: US 2022/0031962 A1
(43) Pub. Date: Feb. 3, 2022

(54) Hypodermic Interface Assembly

(71) Applicant: Neogen Corporation, Lansing, MI (US)

(72) Inventors: Gregory S. Hastings, Richmond, KY (US); Bradley E. Galbreath, Lexington, KY (US); Nicholas J. Wagner, Lexington, KY (US); Taylor Kopacka Leigh, Alpharetta, GA (US); Peter Wyndham Shipp, JR., Woodstock, GA (US); Jason Lye, Atlanta, GA (US); Randall M. Bachtel, Lawrenceville, GA (US); Dexter E. Jacobs, Austin, TX (US); Michael Trovato, Portland, OR (US)

(21) Appl. No.: 16/947,306

(22) Filed: Jul. 28, 2020

(51) Int. Cl. A61M 5/32; A61M 5/34; A61M 5/31

(52) U.S. Cl. CPC A61M 5/3293; A61M 2005/3142; A61M 5/3134; A61M 5/349

(57) ABSTRACT

A hypodermic interface assembly includes a cannula and a hub. The hub is defined by a first portion and a second portion. The second portion of the hub is breakably-connected to the first portion of the hub. The cannula is joined to the second portion of the hub. The second portion of the hub is defined by an outer head surface portion having a first geometry. The second portion of the hub also includes an outer neck or groove surface portion having a second geometry. The first geometry of the outer head surface portion is greater than the second geometry of the outer neck or groove surface portion.

### TECHNICAL FIELD

The disclosure relates generally to hypodermic interface assemblies.

### BACKGROUND

This section provides background information related to the present disclosure and is not necessarily prior art.

While known hypodermic interface assemblies have proven to be acceptable for various applications, such hypodermic interface assemblies are nevertheless susceptible to improvements that may enhance their overall performance and cost. Therefore, a need exists to develop hypodermic interface assemblies that advance the art.

### SUMMARY

This section provides a general summary of the disclosure, and is not a comprehensive disclosure of its full scope or all of its features.

An embodiment of the present invention is a hypodermic interface assembly including a cannula and a hub, wherein the hub is defined by a first portion and a second portion, the second portion of the hub is breakably-connected to the first portion of the hub, the cannula is joined to the second portion of the hub, the second portion of the hub is defined by an outer head surface portion having a first geometry, the second portion of the hub is defined by an outer neck surface portion having a second geometry, and the first geometry of the outer head surface portion is greater than the second geometry of the outer neck or groove surface portion. Further, the outer neck surface portion may define a substantially cylindrical neck portion, the outer neck surface portion may define a substantially conical neck portion, and/or the outer neck surface portion may define a first surface portion that contributes to forming a V-shaped groove.

In another embodiment of the hypodermic interface assembly the cannula may be disposed within a hub passage extending through the first portion and the second portion of the hub; and the hub passage may include a first hub passage portion extending through the first portion of the hub, and a second hub passage portion extending through the second portion of the hub, an outer side surface of the cannula may be secured to an inner surface portion of an inner surface of the hub that defines the hub passage, and the inner surface portion that is defined by the inner surface of the hub may extend through the second portion of the hub.

In other embodiments of the hypodermic interface assembly (a) a proximal end of the cannula may be arranged within and extend into the first hub passage portion extending through the first portion of the hub, (b) all of the second hub passage portion extending through the second portion of the hub may contain a portion of a length of the cannula that extends from a proximal end of the cannula, and/or (c) a portion of the second hub passage portion extending through the second portion of the hub may contain a portion of a length of the cannula that extends from a proximal end of the cannula.

An embodiment of the present invention is a hypodermic interface assembly that includes: (a) a first hypodermic interface assembly portion that is defined by a first portion of a hub body of a hub; and (b) a second hypodermic interface assembly portion that is frangibly-connected to and separable from the first hypodermic interface assembly portion, wherein the second hypodermic interface assembly portion is defined by (i) a cannula and (ii) a second portion of the hub body of the hub, and the cannula is joined to the second portion of the hub body of the hub.

In yet other embodiments of the hypodermic interface assembly, the second portion of the hub body of the hub may be defined by an outer head surface portion having a first geometry and an outer neck surface portion having a second geometry. Further, (a) the first geometry of the outer head surface portion may be greater than the second geometry of the outer neck or groove surface portion; (b) the outer neck surface portion may define a substantially cylindrical neck portion; (c) the outer neck surface portion may define a substantially conical neck portion; or (d) the outer neck surface portion may define a first surface portion that contributes to forming a V-shaped groove. Yet even further, a length of the outer neck surface portion may be defined by a ratio ranging between approximately 0.10 and 4 times the first geometry of the outer head surface portion; or a thickness of a neck of the hub may be defined by a ratio ranging between approximately 0.10 and 1.50 times the first geometry of the outer head surface portion.

In another embodiment, the cannula may be disposed within a hub passage extending through the hub body of the hub, and the hub passage may include: (a) a first hub passage portion extending through the first portion of the hub body of the hub; and (b) a second hub passage portion extending through the second portion of the hub body of the hub, an outer side surface of the cannula may be secured to an inner surface portion of an inner surface of the hub body of the hub that defines the hub passage, and the inner surface portion that is defined by the inner surface of the hub body of the hub may extend through the second portion of the hub body of the hub.

In a further embodiment: a proximal end of the cannula may arranged within and extend into the first hub passage portion extending through the first portion of the hub body of the hub; all of the second hub passage portion extending through the second portion of the hub body of the hub may contain a portion of a length of the cannula that extends from a proximal end of the cannula; or a portion of the second hub passage portion extending through the second portion of the hub body of the hub may contain a portion of a length of the cannula that extends from a proximal of the cannula.

A further embodiment of the invention is a hypodermic interface assembly including a cannula and a hub, wherein the hub may be defined by a first portion and a second portion, the second portion of the hub may be breakably-connected to the first portion of the hub, the cannula may be joined to the second portion of the hub, the second portion of the hub may be defined by an outer head surface portion having a first geometry, and the second portion of the hub may be separated from the a first portion of the hub by a groove surface portion having a second geometry that is less than a portion of the first geometry of the outer head surface portion. Further, a depth of the groove surface portion may be defined by a ratio ranging between approximately 0.15 and 0.95 times the first geometry of the outer head surface portion.

Another aspect of the invention is a method that includes: (a) providing a hub that may be defined by a first portion and a second portion, wherein the second portion of the hub may be breakably-connected to the first portion of the hub; and (b) non-separably joining a cannula to the second portion of the hub. In another aspect, the method may also include separably joining the first portion of the hub to an injection gun; and inserting the cannula into the flesh of a subject. Further, one or both of the cannula and the hub may be subjected to one or more radial forces relative to a central axis extending through the cannula and the hub for mechanically-separating the first portion of the hub and the second portion of the hub; the first portion of the hub may remain separably joined to the injection gun; the cannula may be removably disposed within the flesh of the subject; and the second portion of the hub may be disposed adjacent an outer surface of the flesh of the subject.

Yet another aspect of the inventive method includes locating the second portion of the hub) that is disposed adjacent the outer surface of the flesh of the subject, grasping the second portion of the hub that is disposed adjacent the outer surface of the flesh of the subject, and applying a force to the second portion of the hub to remove the cannula from the flesh of the subject. A further method step may include separating the first portion of the hub from the injection gun.

The details of one or more implementations of the disclosure are set forth in the accompanying drawings and the description below. Other aspects, features, and advantages will be apparent from the description and drawings, and from the claims.

### DESCRIPTION OF DRAWINGS

The drawings described herein are for illustrative purposes only of selected configurations and not all possible implementations, and are not intended to limit the scope of the present disclosure.

FIG. 1 is an exploded perspective view of an exemplary hypodermic interface assembly.

FIG. 2 is a perspective view of an exemplary cannula of the hypodermic interface assembly of FIG. 1.

FIG. 3 is a front perspective view of an exemplary hub of the hypodermic interface assembly of FIG. 1.

FIG. 4 is a rear perspective view of the hub of FIG. 2.

FIG. 5 is another front perspective view of the hub of FIG. 2.

FIG. 6 is a side view of the hub of FIG. 2.

FIG. 7 is a cross-sectional view of the hub according to line 7-7 of FIG. 6.

FIG. 8A is another side view of the hub of FIG. 2.

FIG. 8B is a top view of the hub according to arrow 8B of FIG. 8A.

FIG. 9A is another side view of the hub of FIG. 2.

FIG. 9B is a top view of the hub according to arrow 9B of FIG. 9A.

FIG. 10 is a bottom view of the hub according to arrow 10 of FIG. 8A or 9A.

FIG. 11A is a cross-sectional view of a partially assembled hypodermic interface assembly arranged in a first partially assembly state according to line 11-11 of FIG. 1.

FIG. 11B is a cross-sectional view of a partially assembled hypodermic interface assembly arranged in a second partially assembly state according to line 19-19 of FIG. 1.

FIG. 11C is a cross-sectional view of an assembled hypodermic interface assembly according to line 11′-11′ of any of FIG. 12.

FIG. 12 is an assembled front perspective view of the hypodermic interface assembly of FIG. 1.

FIG. 13 is a top view of the hypodermic interface assembly according to arrow 13 of FIG. 12.

FIG. 14 is a bottom view of the hypodermic interface assembly according to arrow 14 of FIG. 12.

FIG. 15 is an assembled rear perspective view of the hypodermic interface assembly of FIG. 1.

FIG. 16 is another assembled front perspective view of the hypodermic interface assembly of FIG. 1.

FIG. 17 is a side view of the hypodermic interface assembly of FIG. 1.

FIG. 18A is perspective cross-sectional view according to line 18-18 of the front perspective view of the hypodermic interface assembly of FIG. 16 that is arranged in an at-rest orientation.

FIG. 18B is side cross-sectional view of the assembled hypodermic interface assembly according to arrow 18B of FIG. 18A.

FIG. 19A is another perspective cross-sectional view according to FIG. 18A that is arranged in a biased orientation.

FIG. 19B is side cross-sectional view of the assembled hypodermic interface assembly according to arrow 19B of FIG. 19A.

FIG. 20A is another perspective cross-sectional view according to FIG. 19A that is arranged in a separated orientation.

FIG. 20B is side cross-sectional view of the assembled hypodermic interface assembly according to arrow 20B of FIG. 20A.

FIG. 21A is an enlarged view of the assembled hypodermic interface assembly according to line 21A of FIG. 18B.

FIG. 21B is a reduced size view of another assembled hypodermic interface assembly according to FIG. 21A.

FIG. 21C is a reduced size view of another assembled hypodermic interface assembly according to FIG. 21A.

FIG. 21D is a reduced size view of another assembled hypodermic interface assembly according to FIG. 21A.

FIG. 21E is a reduced size view of another assembled hypodermic interface assembly according to FIG. 21A.

FIG. 21F is a reduced size view of another assembled hypodermic interface assembly according to FIG. 21A.

FIG. 21G is a reduced size view of another assembled hypodermic interface assembly according to FIG. 21A.

FIG. 22 is an exploded perspective view of an exemplary hypodermic interface assembly.

FIG. 23 is a front perspective view of an exemplary hub of the hypodermic interface assembly of FIG. 22.

FIG. 24 is a rear perspective view of the hub of FIG. 23.

FIG. 25 is a side view of the hub of FIG. 23.

FIG. 26 is a cross-sectional view of the hub according to line 26-26 of FIG. 25.

FIG. 27 is an assembled front perspective view of the hypodermic interface assembly of FIG. 22.

FIG. 28 is perspective cross-sectional view according to line 28-28 of the assembled front perspective view of the hypodermic interface assembly of FIG. 27 that is arranged in an at-rest orientation.

FIG. 29 is perspective cross-sectional view according to FIG. 28 that is arranged in a separated orientation.

FIG. 30A is an enlarged view of the assembled hypodermic interface assembly of FIG. 28 that is arranged in the at-rest orientation.

FIG. 30B is a reduced size view of another assembled hypodermic interface assembly according to FIG. 30A.

FIG. 30C is a reduced size view of another assembled hypodermic interface assembly according to FIG. 30A.

FIG. 30D is a reduced size view of another assembled hypodermic interface assembly according to FIG. 30A.

FIG. 30E is a reduced size view of another assembled hypodermic interface assembly according to FIG. 30A.

FIG. 30F is a reduced size view of another assembled hypodermic interface assembly according to FIG. 30A.

FIG. 30G is a reduced size view of another assembled hypodermic interface assembly according to FIG. 30A.

FIG. 31 is an exploded perspective view of an exemplary hypodermic interface assembly.

FIG. 32 is a front perspective view of an exemplary hub of the hypodermic interface assembly of FIG. 31.

FIG. 33 is a rear perspective view of the hub of FIG. 32.

FIG. 34 is a side view of the hub of FIG. 32.

FIG. 35 is a cross-sectional view of the hub according to line 35-35 of FIG. 34.

FIG. 36 is an assembled front perspective view of the hypodermic interface assembly of FIG. 31.

FIG. 37 is perspective cross-sectional view according to line 37-37 of the assembled front perspective view of the hypodermic interface assembly of FIG. 36 that is arranged in an at-rest orientation.

FIG. 38 is perspective cross-sectional view according to FIG. 37 that is arranged in a separated orientation.

FIG. 39A is an enlarged view of the assembled hypodermic interface assembly of FIG. 37 that is arranged in the at-rest orientation.

FIG. 39B is a reduced size view of another assembled hypodermic interface assembly according to FIG. 39A.

FIG. 39C is a reduced size view of another assembled hypodermic interface assembly according to FIG. 39A.

FIG. 39D is a reduced size view of another assembled hypodermic interface assembly according to FIG. 39A.

FIG. 39E is a reduced size view of another assembled hypodermic interface assembly according to FIG. 39A.

FIG. 39F is a reduced size view of another assembled hypodermic interface assembly according to FIG. 39A.

FIG. 39G is a reduced size view of another assembled hypodermic interface assembly according to FIG. 39A.

FIG. 40 is an exploded perspective view of an exemplary hypodermic interface assembly.

FIG. 41 is a front perspective view of an exemplary hub of the hypodermic interface assembly of FIG. 40.

FIG. 42 is a rear perspective view of the hub of FIG. 41.

FIG. 43 is a side view of the hub of FIG. 41.

FIG. 44 is a cross-sectional view of the hub according to line 44-44 of FIG. 43.

FIG. 45 is an assembled front perspective view of the hypodermic interface assembly of FIG. 40.

FIG. 46 is perspective cross-sectional view according to line 46-46 of the assembled front perspective view of the hypodermic interface assembly of FIG. 45 that is arranged in an at-rest orientation.

FIG. 47 is perspective cross-sectional view according to FIG. 46 that is arranged in a separated orientation.

FIG. 48A is an enlarged view of the assembled hypodermic interface assembly of FIG. 46 that is arranged in the at-rest orientation.

FIG. 48B is a reduced size view of another assembled hypodermic interface assembly according to FIG. 48A.

FIG. 48C is a reduced size view of another assembled hypodermic interface assembly according to FIG. 48A.

FIG. 48D is a reduced size view of another assembled hypodermic interface assembly according to FIG. 48A.

FIG. 48E is a reduced size view of another assembled hypodermic interface assembly according to FIG. 48A.

FIG. 48F is a reduced size view of another assembled hypodermic interface assembly according to FIG. 48A.

FIG. 48G is a reduced size view of another assembled hypodermic interface assembly according to FIG. 48A.

FIG. 49 is an exploded perspective view of an exemplary hypodermic interface assembly.

FIG. 50 is a front perspective view of an exemplary hub of the hypodermic interface assembly of FIG. 49.

FIG. 51 is a rear perspective view of the hub of FIG. 50.

FIG. 52 is a side view of the hub of FIG. 50.

FIG. 53 is a cross-sectional view of the hub according to line 53-53 of FIG. 52.

FIG. 54 is an assembled front perspective view of the hypodermic interface assembly of FIG. 49.

FIG. 55 is perspective cross-sectional view according to line 55-55 of the assembled front perspective view of the hypodermic interface assembly of FIG. 54 that is arranged in an at-rest orientation.

FIG. 56 is perspective cross-sectional view according to FIG. 55 that is arranged in a separated orientation.

FIG. 57A is an enlarged view of the assembled hypodermic interface assembly of FIG. 55 that is arranged in the at-rest orientation.

FIG. 57B is a reduced size view of another assembled hypodermic interface assembly according to FIG. 57A.

FIG. 57C is a reduced size view of another assembled hypodermic interface assembly according to FIG. 57A.

FIG. 57D is a reduced size view of another assembled hypodermic interface assembly according to FIG. 57A.

FIG. 57E is a reduced size view of another assembled hypodermic interface assembly according to FIG. 57A.

FIG. 57F is a reduced size view of another assembled hypodermic interface assembly according to FIG. 57A.

FIG. 57G is a reduced size view of another assembled hypodermic interface assembly according to FIG. 57A.

FIG. 58 is a view of a hypodermic interface assembly arranged proximate animalia.

FIG. 59A is a side view of the hypodermic interface assembly and a cross-sectional view of a portion of the animalia of FIG. 58 arranged in a spaced-apart relationship.

FIG. 59B is another side view of the hypodermic interface assembly and another cross-sectional view of a portion of the animalia according to FIG. 59A arranged in a pierced relationship.

FIG. 59C is another side view of the hypodermic interface assembly and another cross-sectional view of a portion of the animalia according to FIG. 59B arranged in a pierced relationship while, optionally, the hypodermic interface assembly is utilized for injecting a fluid into the animalia.

FIG. 59D is another side view of the hypodermic interface assembly and another cross-sectional view of a portion of the animalia according to FIG. 59B arranged in a pierced-and-torqued relationship.

FIG. 59E is another side view of the hypodermic interface assembly and another cross-sectional view of a portion of the animalia according to FIG. 59D arranged in a separated-after-pierced relationship defining a first portion of the hypodermic interface assembly attached to an injection gun and a second portion of the hypodermic interface assembly impaled within flesh of the animalia.

FIG. 59F is another side view of the according to FIG. 59E illustrating a user grasping the second portion of the hypodermic interface assembly that is impaled within flesh of the animalia.

FIG. 59G is another side view of the according to FIG. 59F illustrating the user removing the second portion of the hypodermic interface assembly that was impaled within flesh of the animalia.

Corresponding reference numerals indicate corresponding parts throughout the drawings.

### DETAILED DESCRIPTION

Example configurations will now be described more fully with reference to the accompanying drawings. Example configurations are provided so that this disclosure will be thorough, and will fully convey the scope of the disclosure to those of ordinary skill in the art. Specific details are set forth such as examples of specific components, devices, and methods, to provide a thorough understanding of configurations of the present disclosure. It will be apparent to those of ordinary skill in the art that specific details need not be employed, that example configurations may be embodied in many different forms, and that the specific details and the example configurations should not be construed to limit the scope of the disclosure.

The figures illustrate exemplary implementations of hypodermic interface assemblies. Based on the foregoing, it is to be generally understood that the nomenclature used herein is simply for convenience and the terms used herein should be given the broadest meaning by one of ordinary skill in the art.

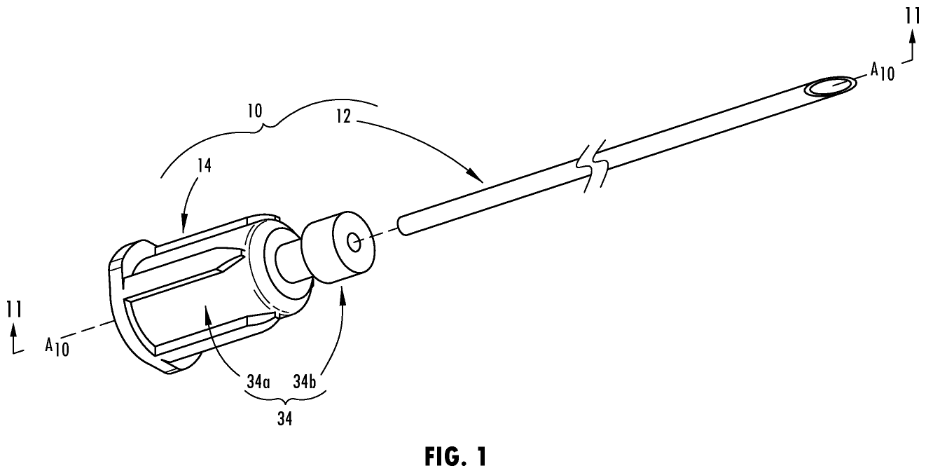

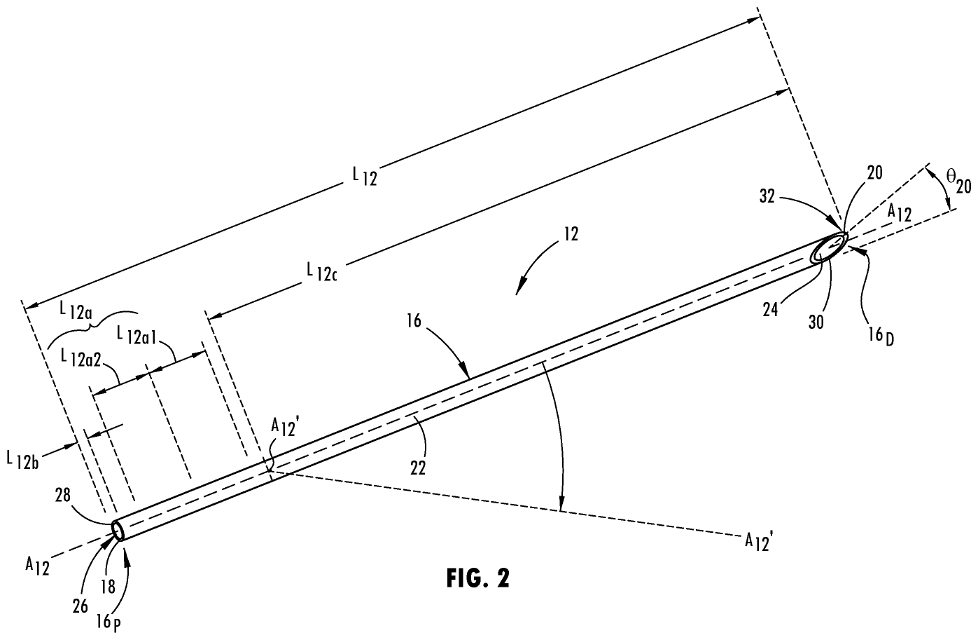

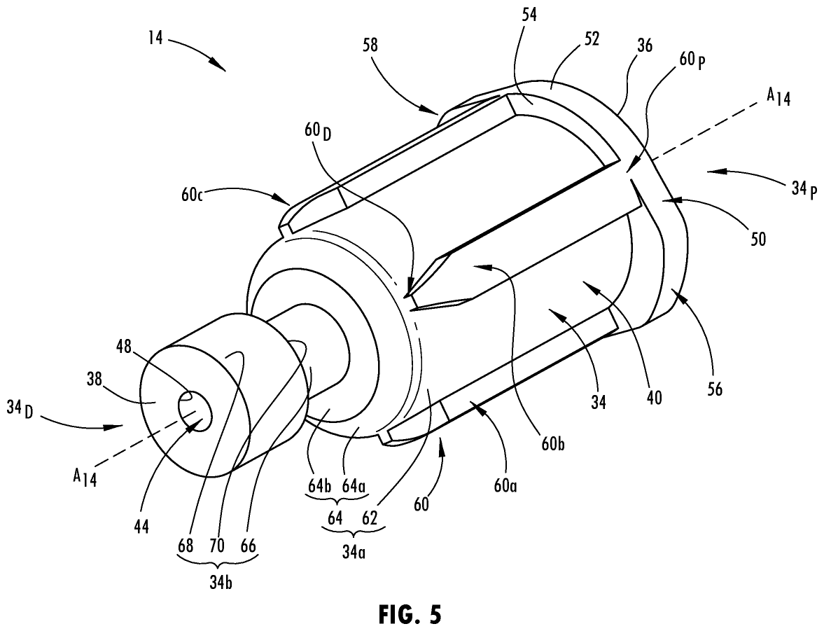

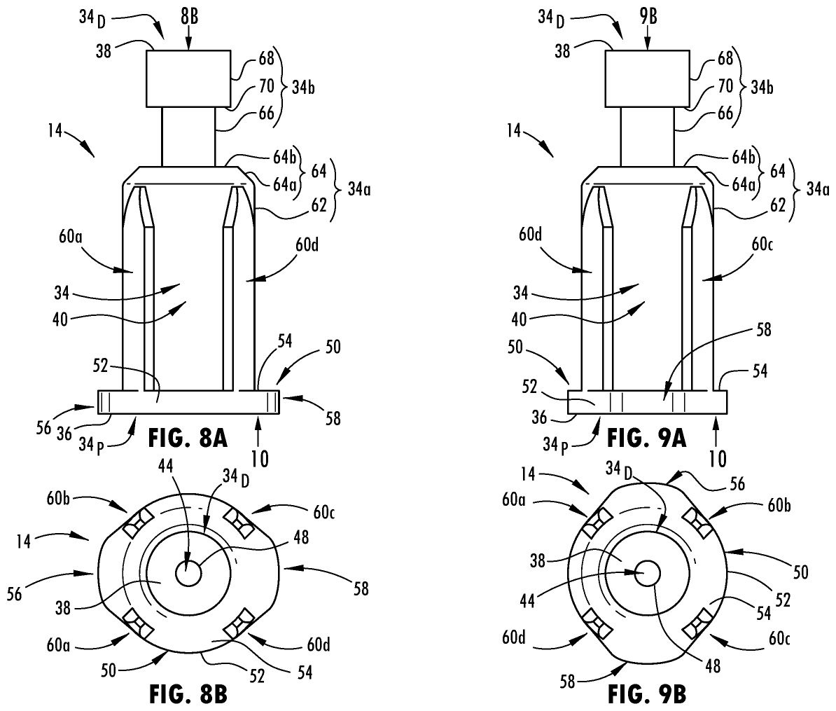

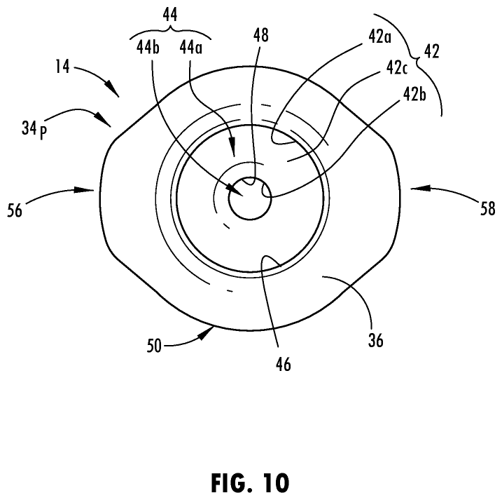

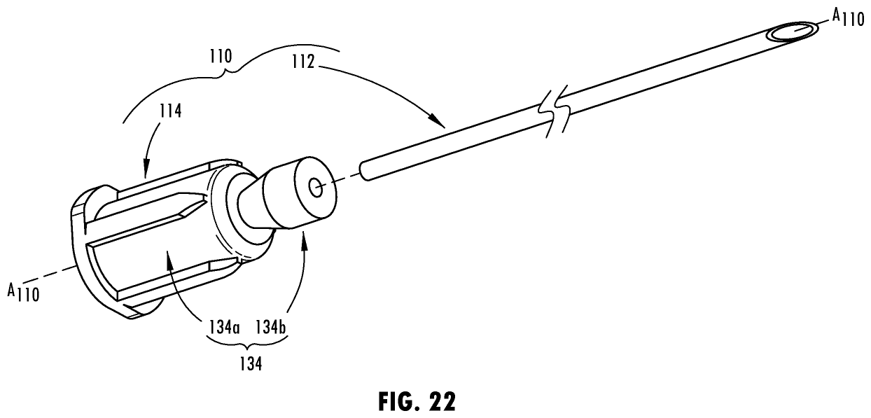

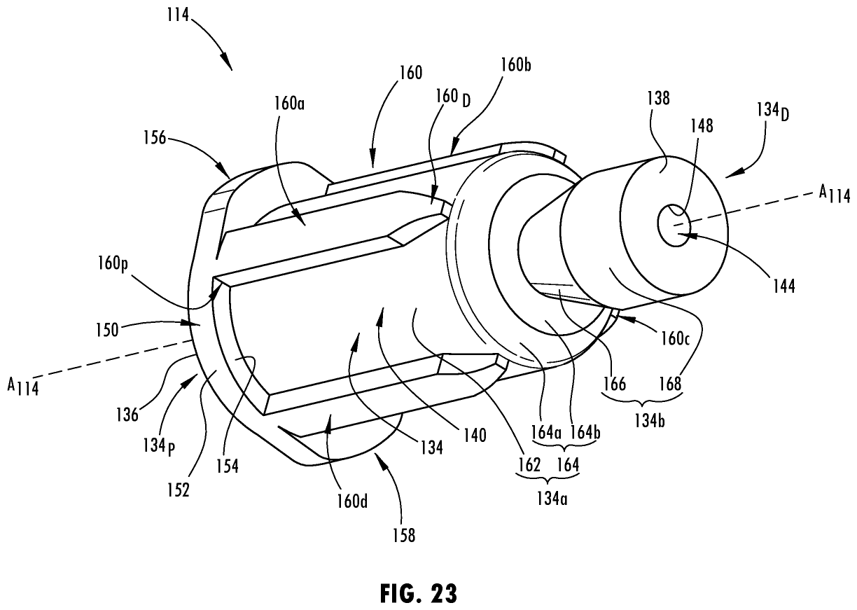

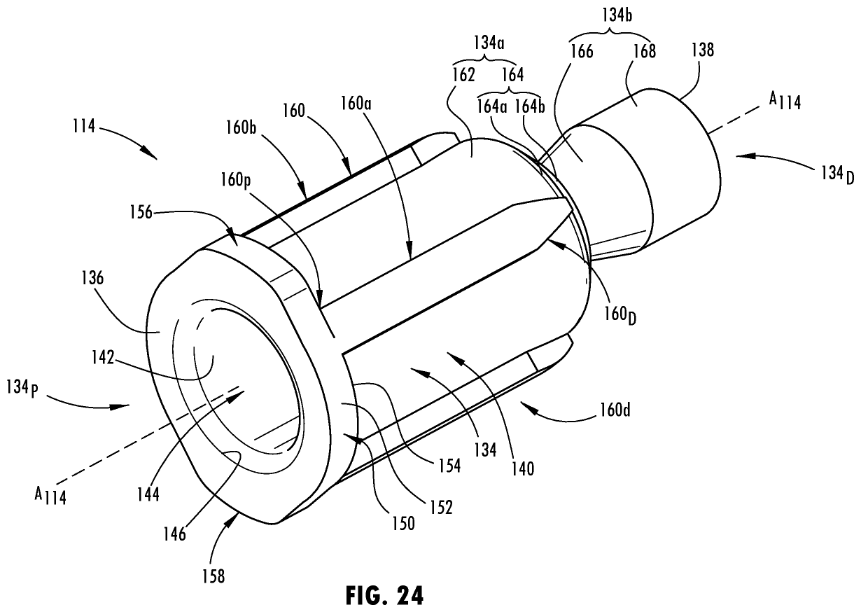

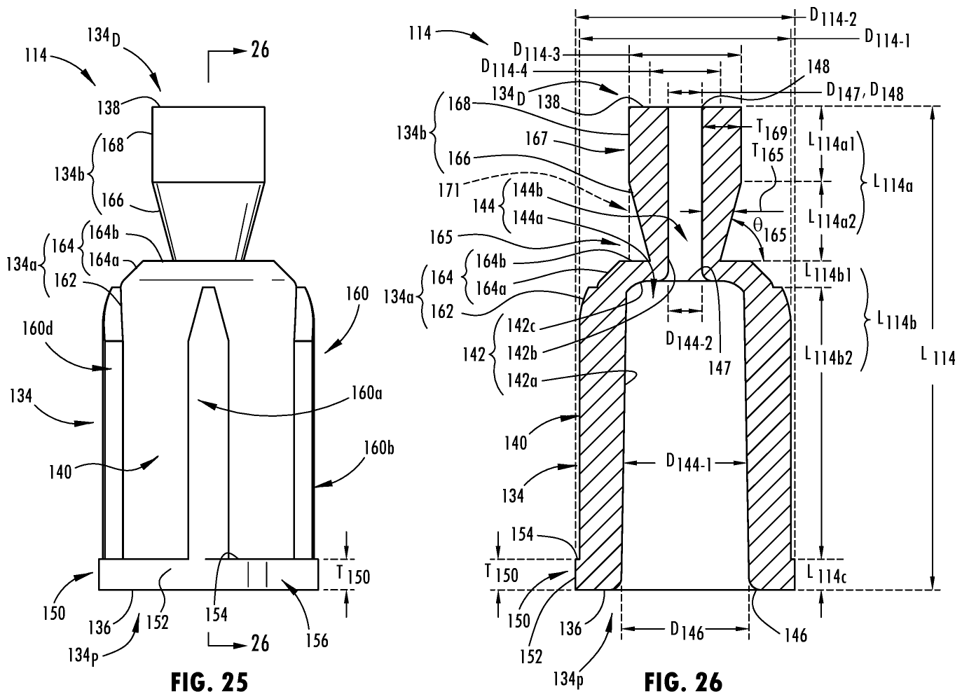

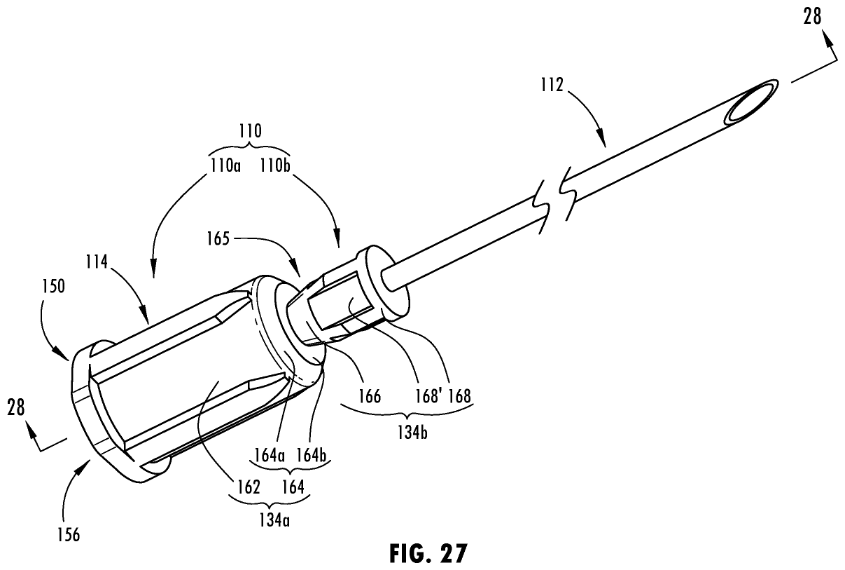

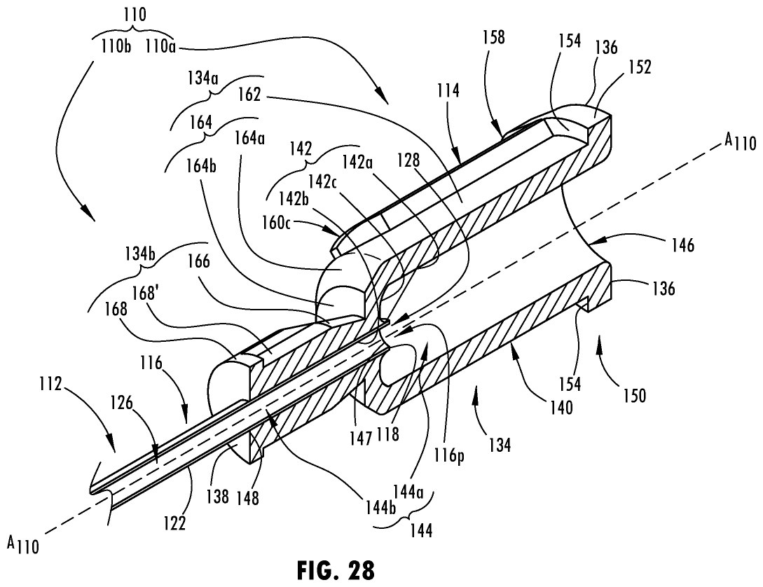

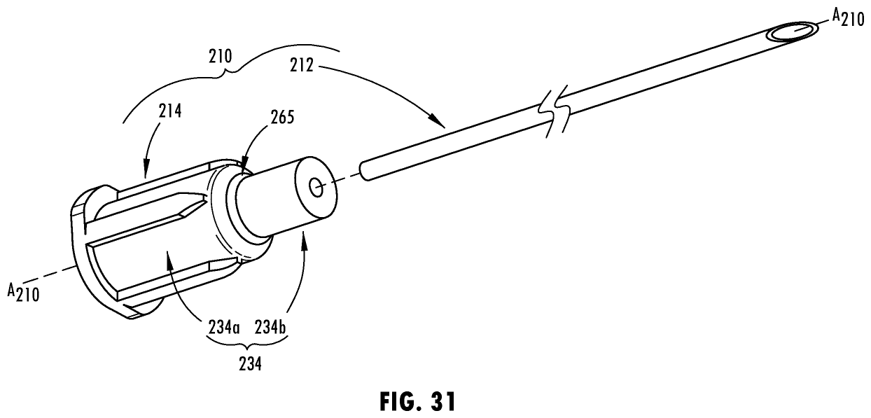

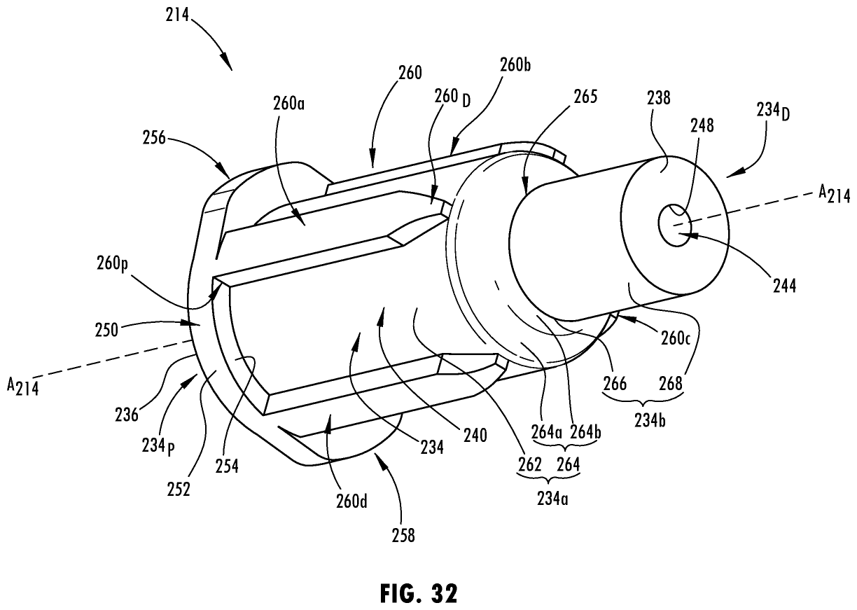

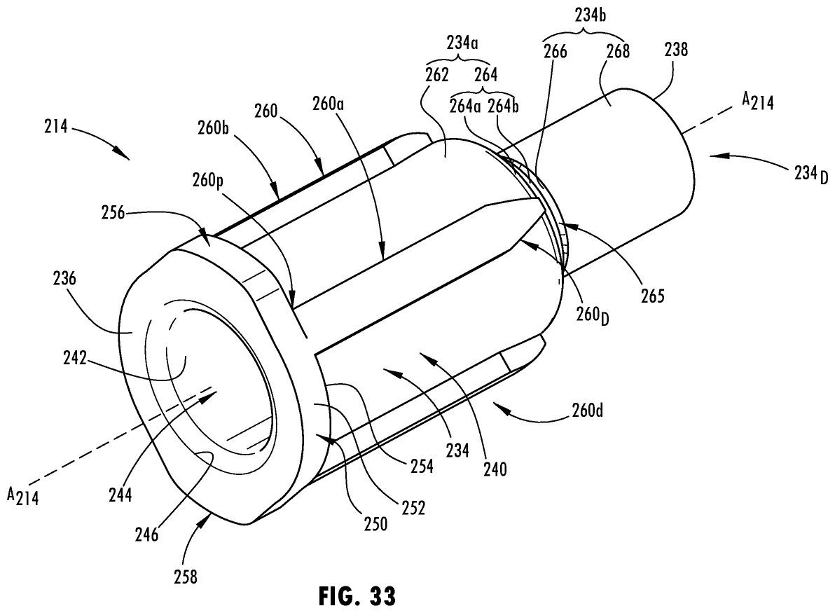

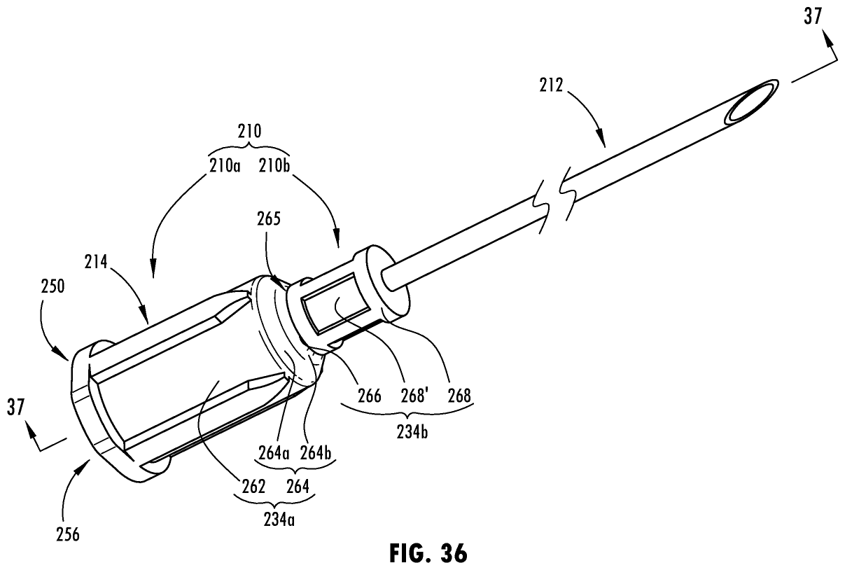

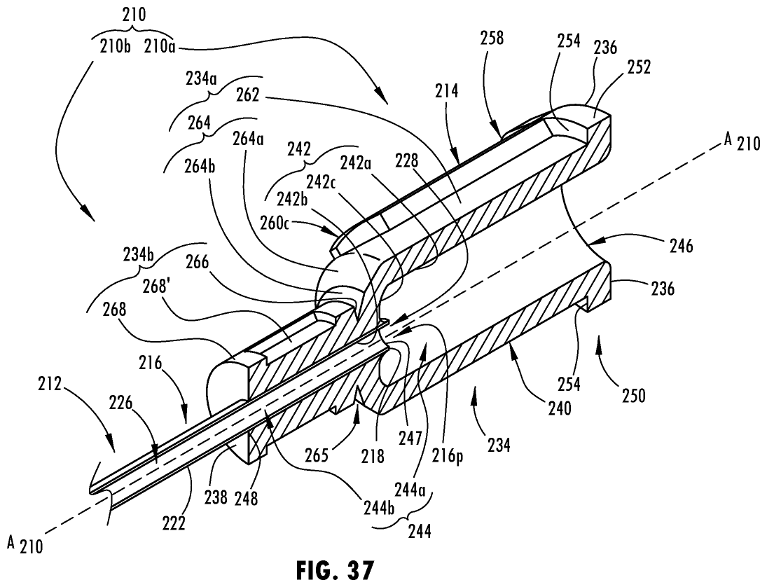

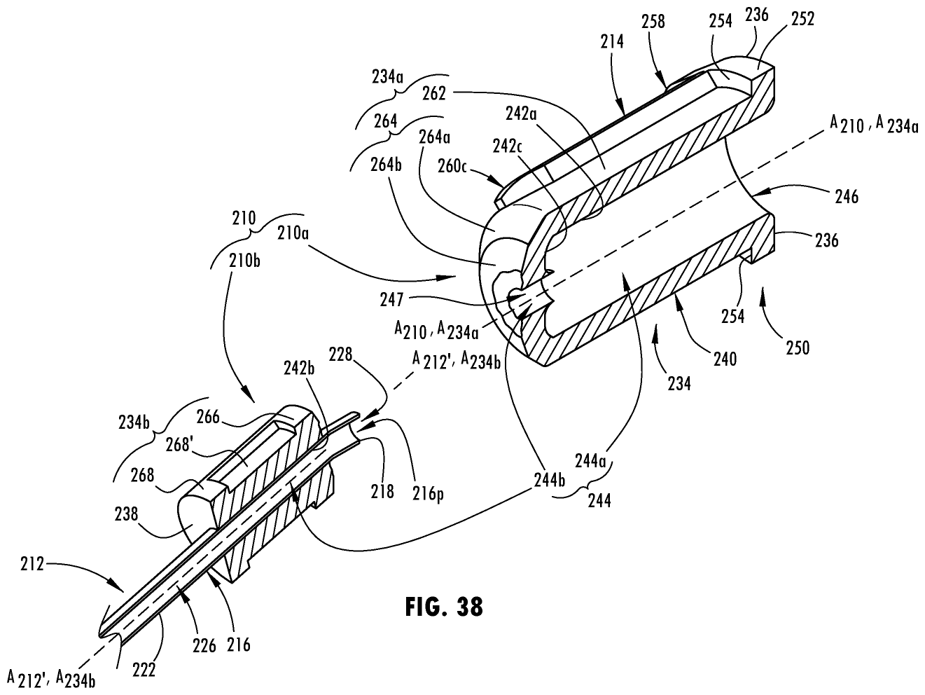

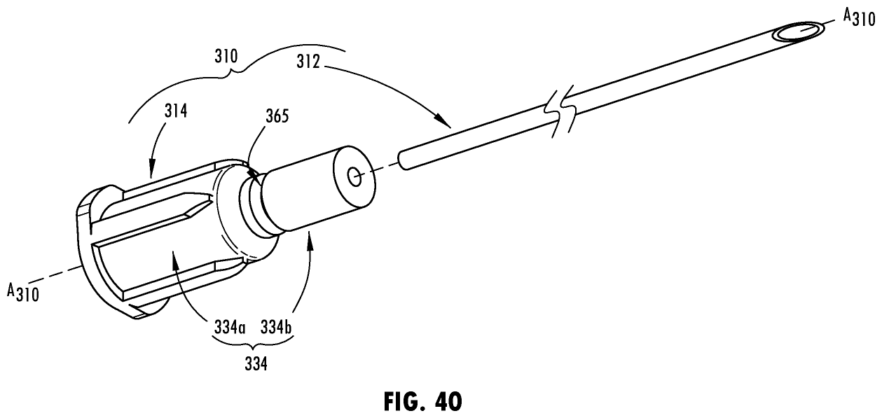

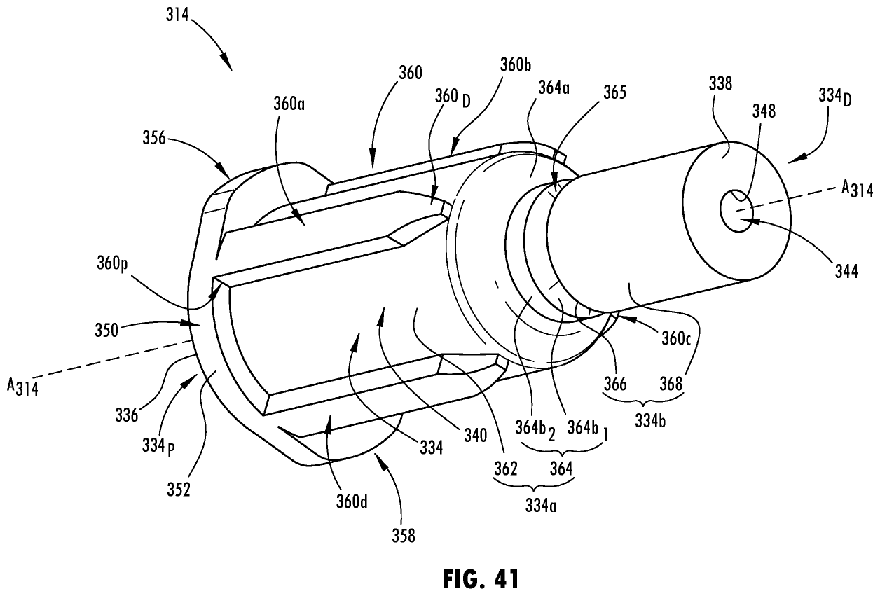

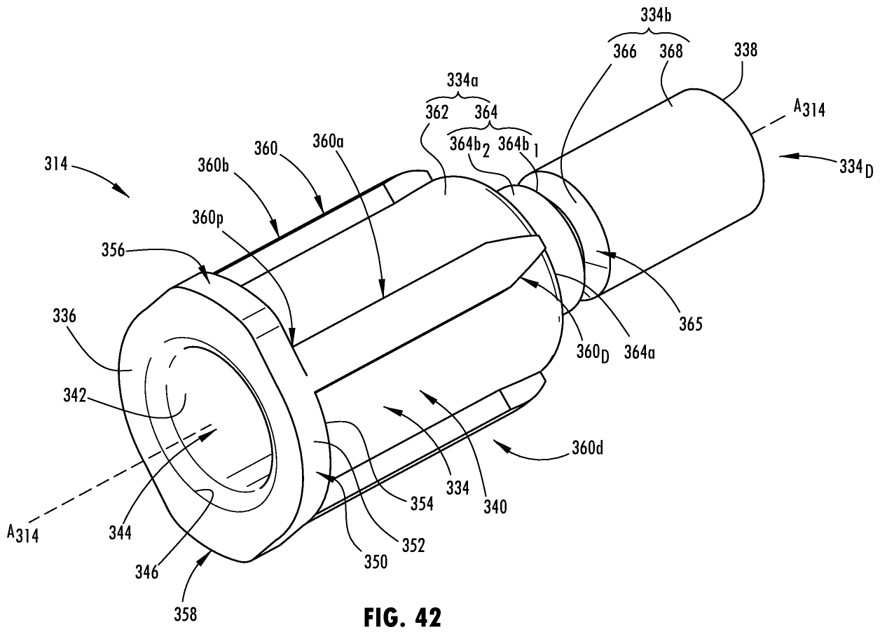

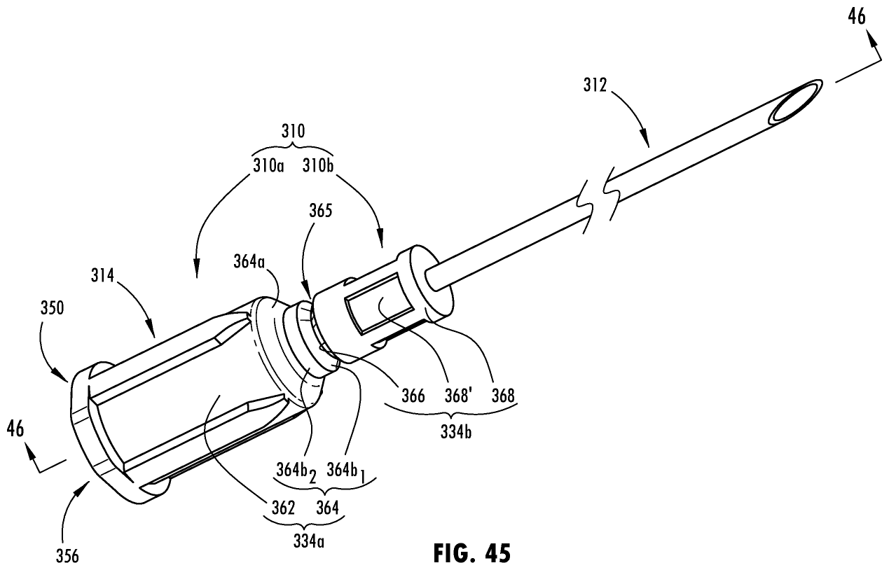

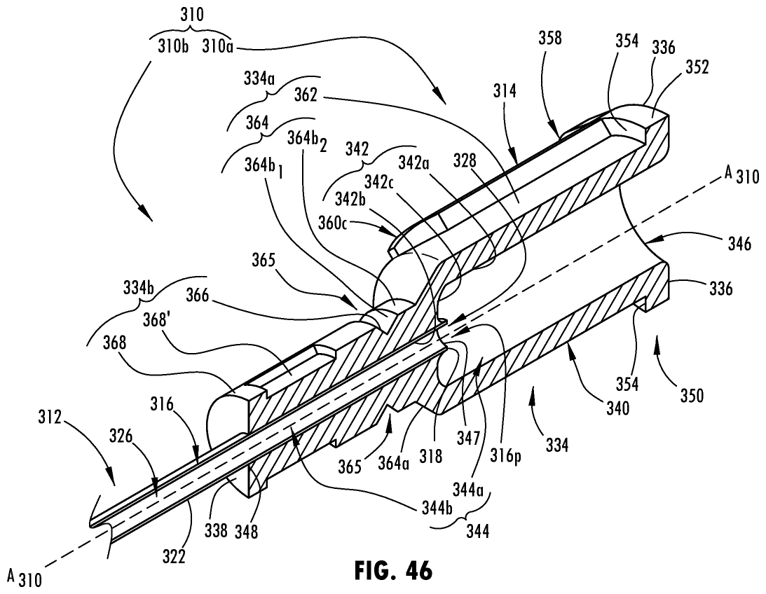

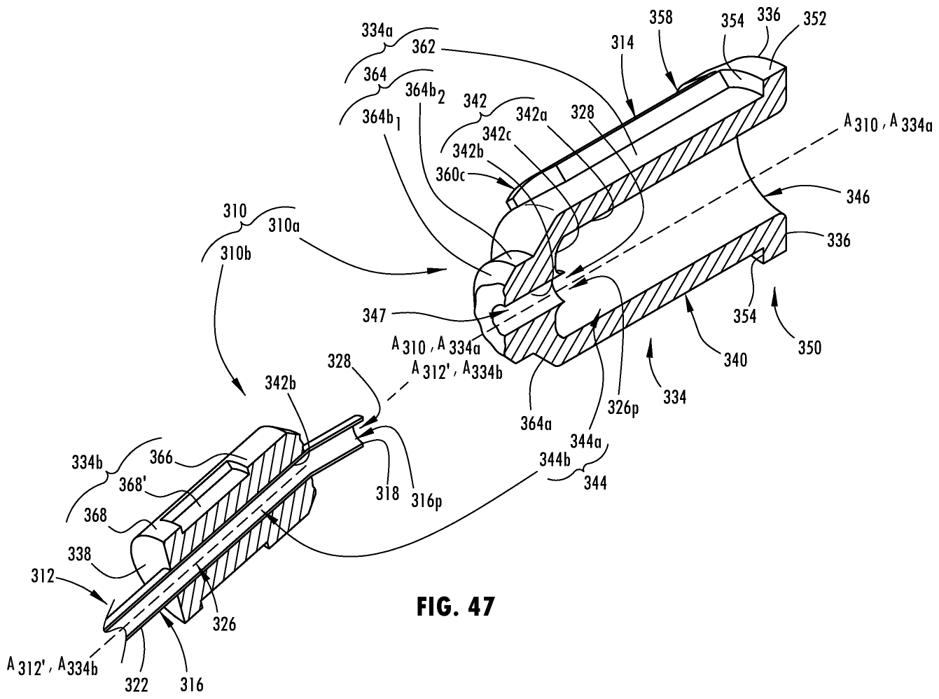

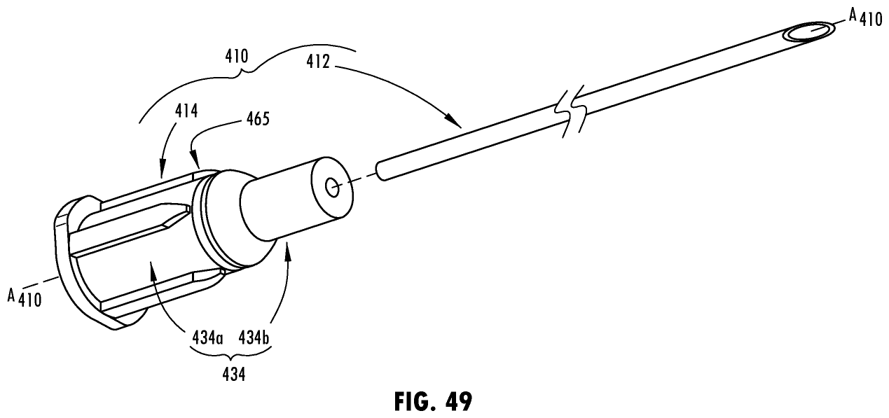

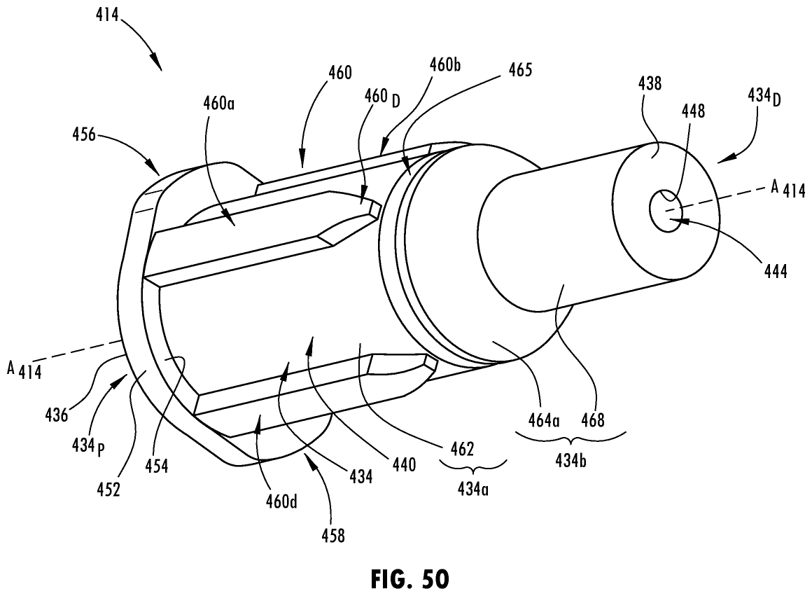

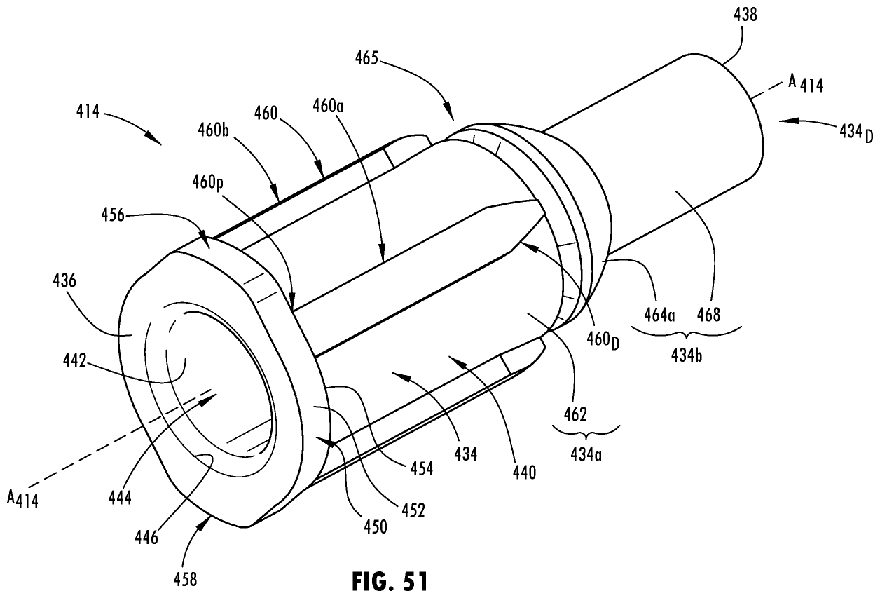

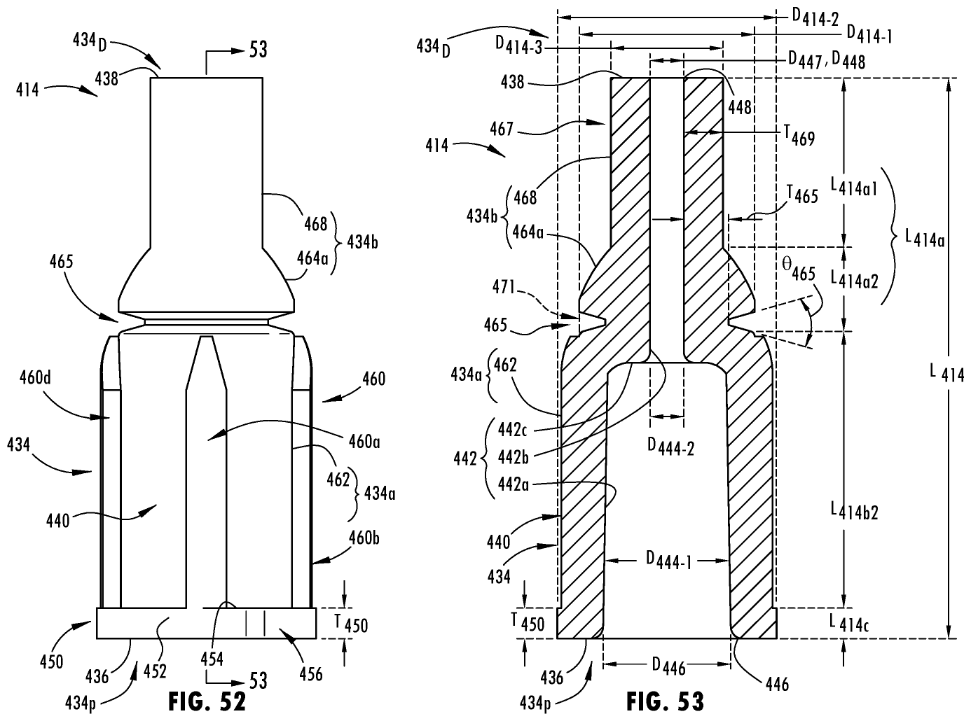

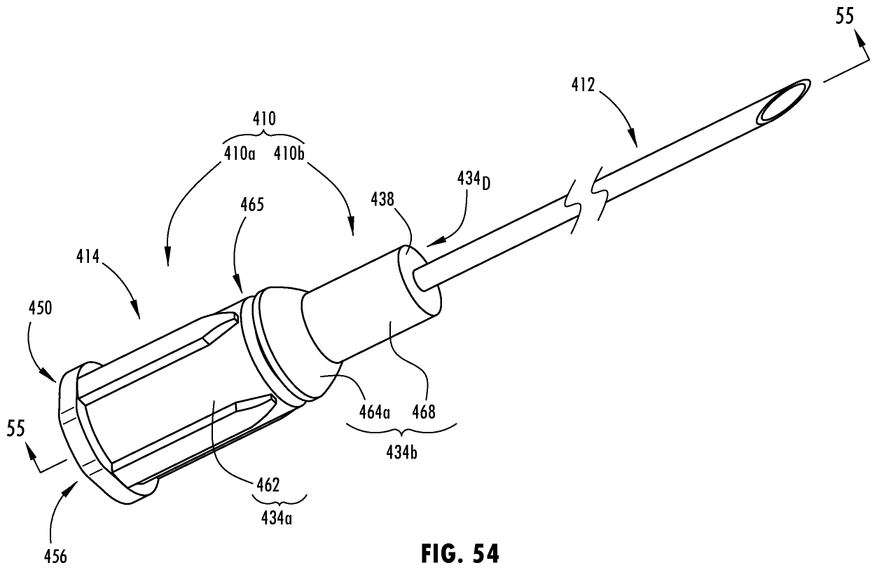

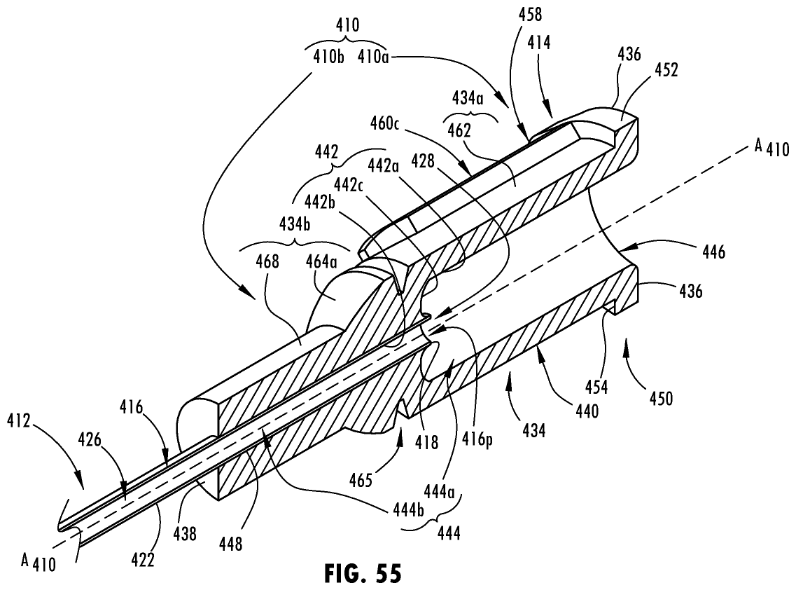

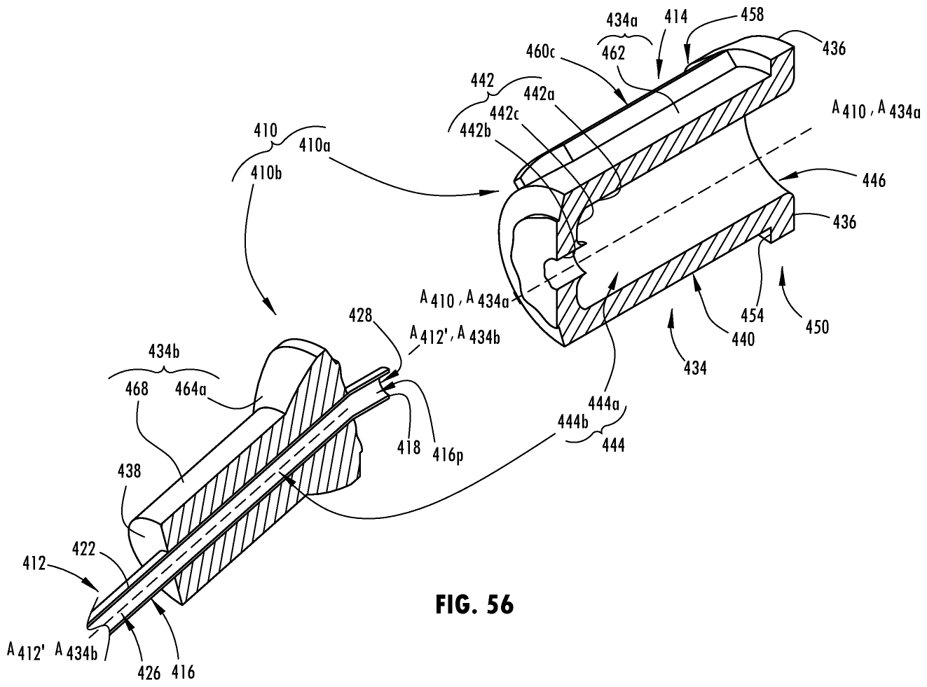

Referring to FIGS. 1,11C, and 12-21A, a hypodermic interface assembly including a cannula 12 (see, e.g., FIGS. 1-2) and a hub 14 (see, e.g., FIGS. 1 and 3-10) is shown generally at 10. A central axis that extends through an axial center of each component (i.e., the cannula 12 and the hub 14) of the hypodermic interface assembly 10 is shown generally at A₁₀-A₁₀. Alternative configurations of the hypodermic interface assembly 10 are shown generally at 110 (see, e.g., FIGS. 22-30), 210 (see, e.g., FIGS. 31-39), 310 (see, e.g., FIGS. 40-48), and 410 (see, e.g., FIGS. 49-57).

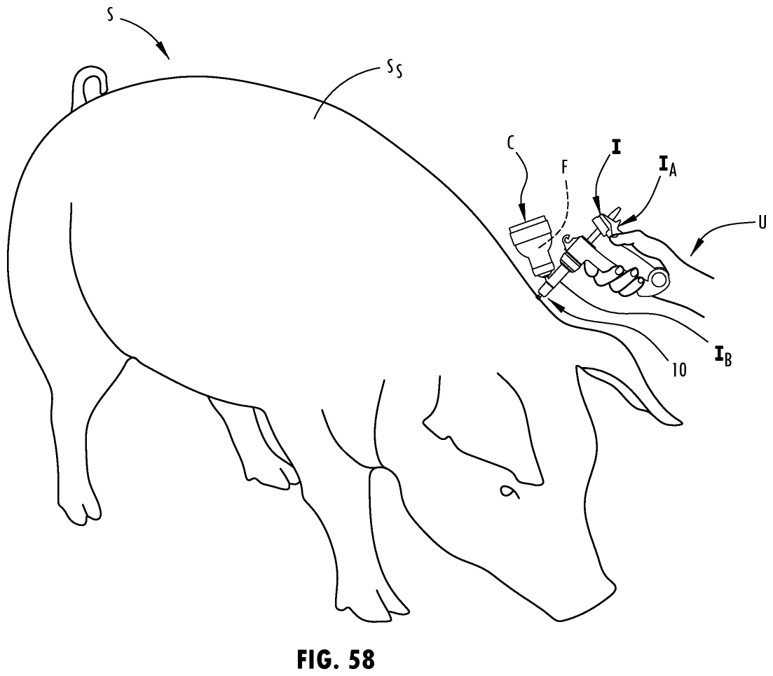

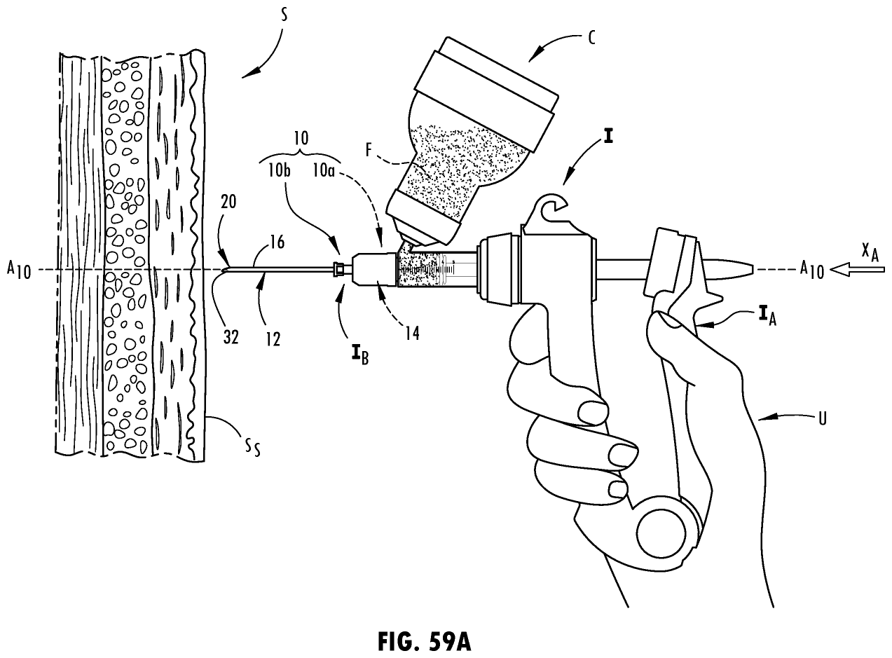

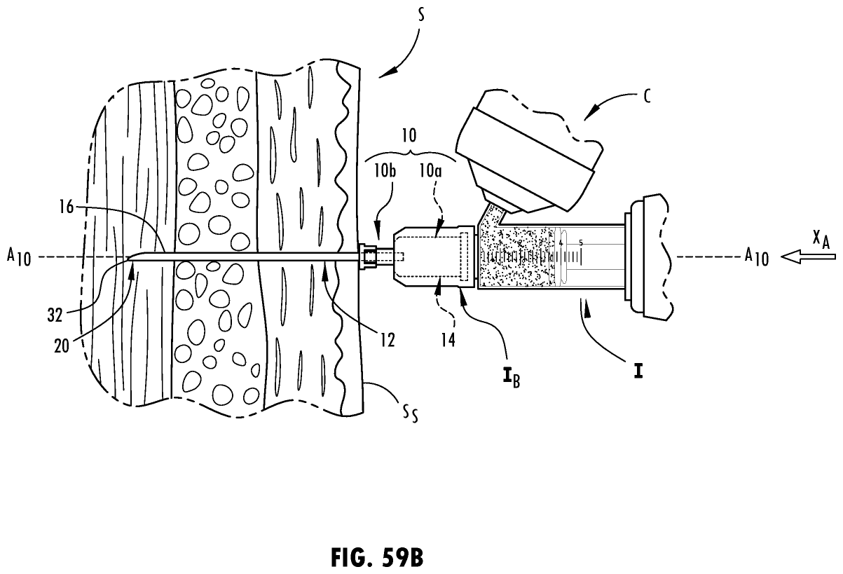

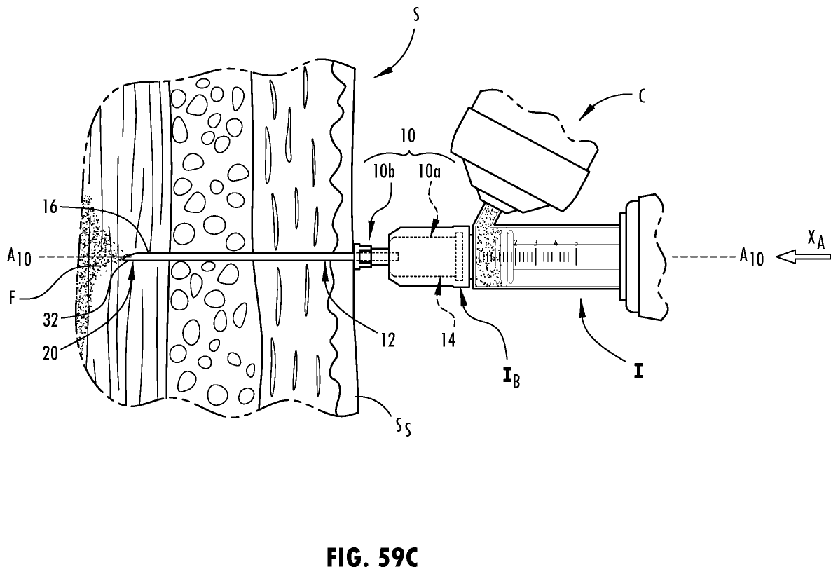

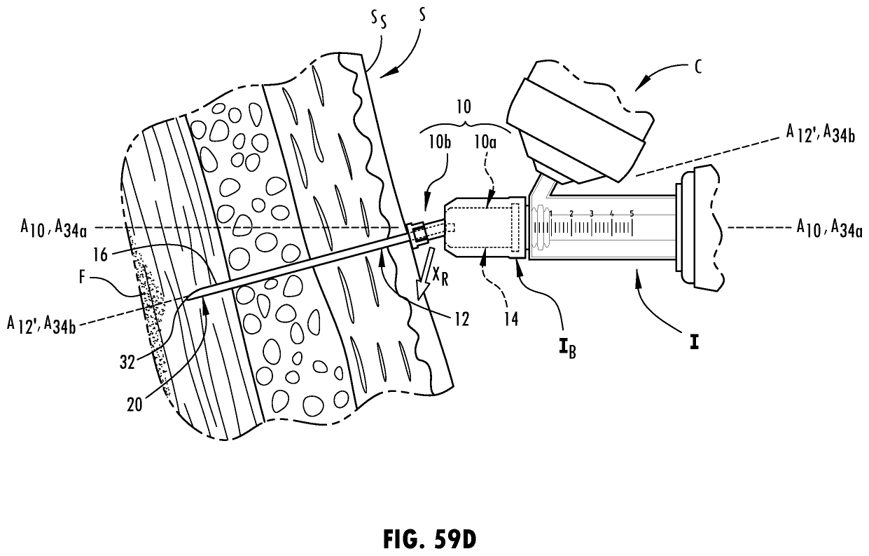

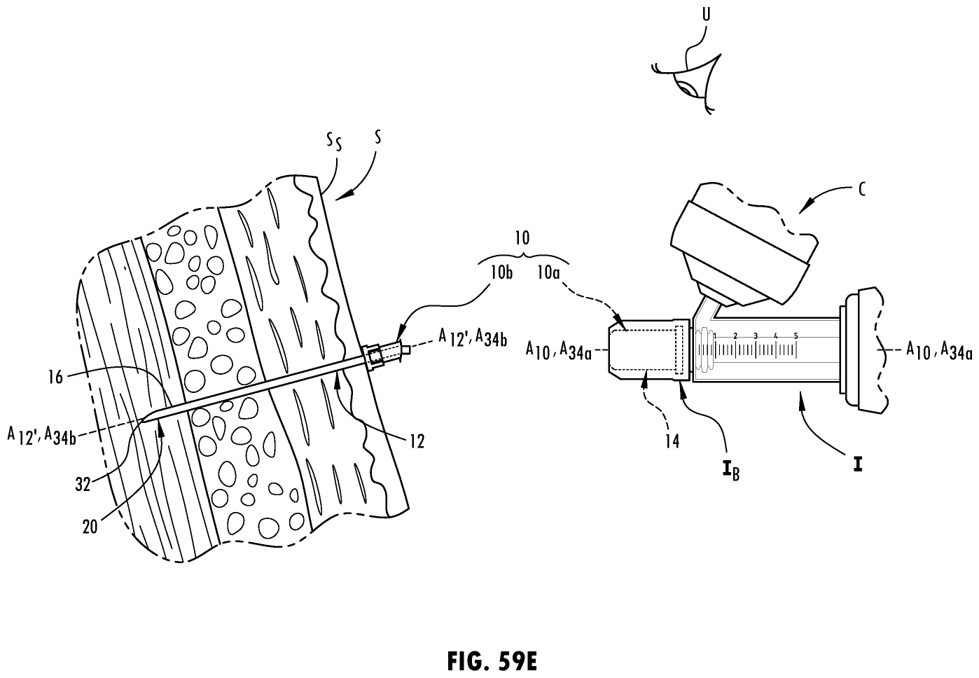

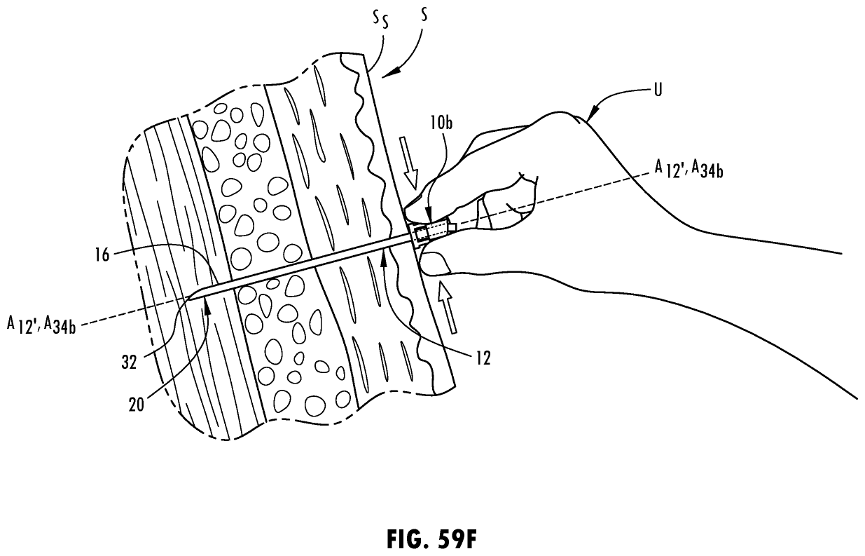

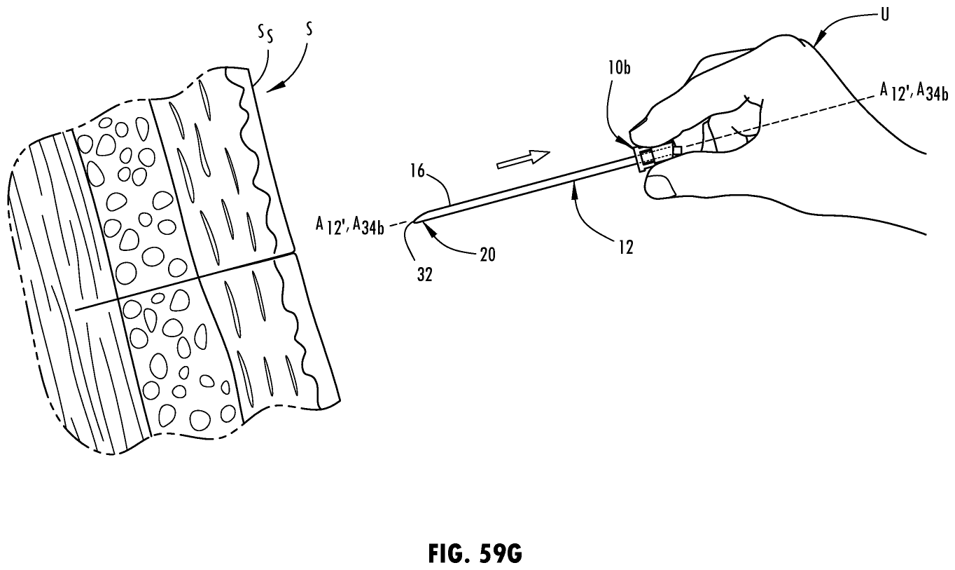

As seen at FIGS. 58 and 59A-59G, the cannula 12 is configured to pierce an outer surface S_{S} (e.g., the skin or hide) of a subject S (e.g., animalia, such as a human or non-human). The purpose of piercing the skin or hide S_{S} of the animalia S may be directed to injecting a fluid F (e.g., a medicament, a pharmaceutical, a vaccine, an anesthetic, or the like) into the animalia S as seen at, for example, FIG. 59C. In other examples, the purpose of piercing the skin or hide S_{S} of the animalia S may be directed to the purpose of drawing a fluid F (e.g., blood) from the animalia S. Accordingly, the cannula 12 may be referred to as a hypodermic cannula, and, as such, the assembly 10 may be referred to as a hypodermic interface assembly as a result of the cannula 12 being capable of injecting or drawing a fluid F into/from the animalia S.

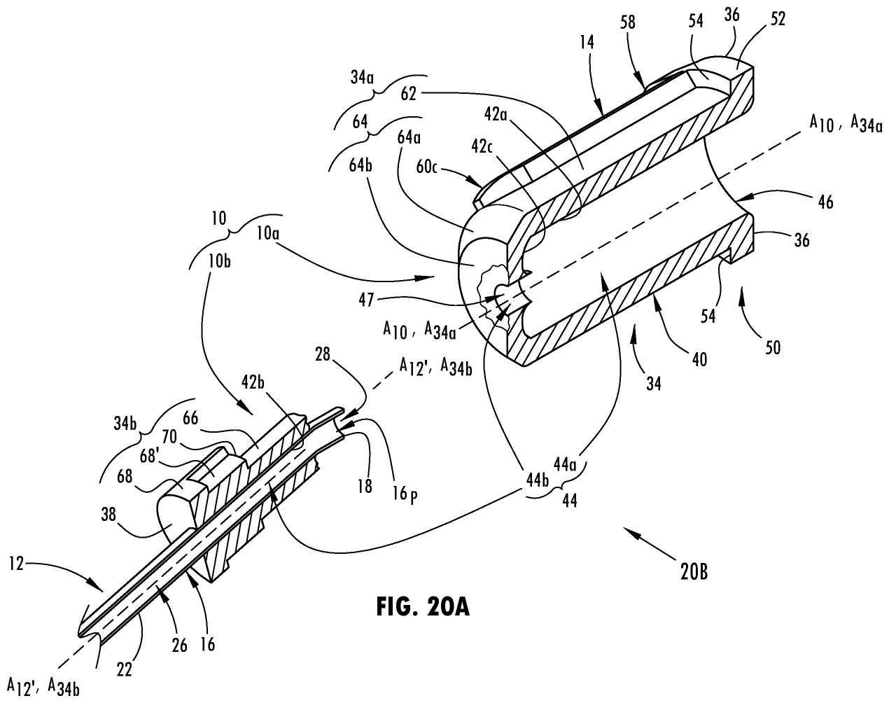

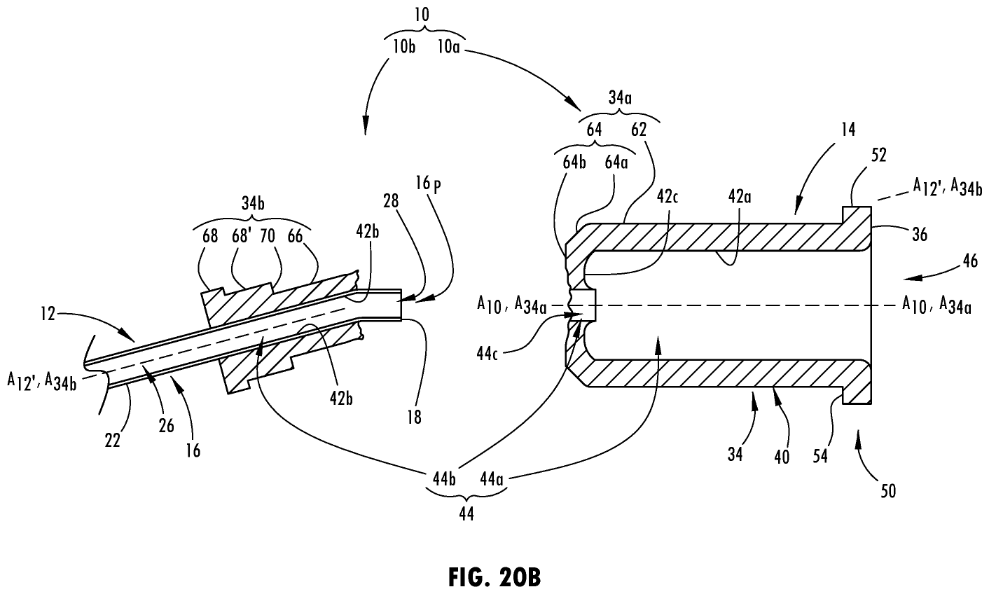

The design of the hypodermic interface assembly 10 (and also the other exemplary hypodermic interface assemblies 110, 210, 310, 410) provides for: (1) a first portion (see, e.g., a first portion 10a at FIGS. 20A-20B and 59E-59G) of the hypodermic interface assembly 10 that is configured to remain attached to an injection gun I after the cannula 12 is subjected to one or more radial forces X_{R} (see, e.g., FIG. 59D) relative to the central axis A₁₀-A₁₀ extending through the hypodermic interface assembly 10; and (2) a second portion (see, e.g., a second portion 10b at FIGS. 20A-20B and 59E-59G) of the hypodermic interface assembly 10 that is configured to predictably and controllably separate from the first portion 10a of the hypodermic interface assembly 10 after the cannula 12 is subjected to the one or more radial forces X_{R} relative to the central axis A₁₀-A₁₀ extending through the hypodermic interface assembly 10. As seen at FIGS. 59E-59Q in some configurations, the second portion 10b of the hypodermic interface assembly 10 includes the entirety of a length (see, e.g., L₁₂ in FIG. 2) of the cannula 12. In some instances, predictable and controlled separation of the second portion 10b of the hypodermic interface assembly 10 from the first portion 10a of the hypodermic interface assembly 10 may occur after the cannula 12 pierces the subject S (see, e.g., FIGS. 58 and 59B-59D). The subject S may be, for example, animalia, such as a human or non-human (i.e., an animal, such as a pig or swine). In other examples, the subject S may be an inanimate object. The predicable and controlled separation of the second portion 10b of the hypodermic interface assembly 10 from the first portion 10a of the hypodermic interface assembly 10 mitigates separation of the cannula 12 from a non-separated, non-broken, or unitary configuration of the hub 14, which may otherwise result in the cannula 12 being broken-off from the injection gun I and subsequently being lost within the flesh of the animalia.

As seen at FIG. 2, the cannula 12 is defined by a tube-shaped body 16 having a proximal end 16_{P} and a distal end 16_{D}. The cannula 12 is defined by a length L₁₂ extending between the proximal end 16_{P} of the tube-shaped body 16 and the distal end 16_{D} of the tube-shaped body 16. The length L₁₂ of the cannula 12 is defined by a plurality of sub-lengths L₁₂ₐ (including sub-length portions L₁₂ₐ₁ and L₁₂ₐ₂), L_{12b}, and L_{12c}, which will be further described in the following disclosure.

The cannula 12 may be formed using any desirable manufacturing procedure such as, for example: a drawing procedure, a molding procedure; a casting procedure; a machining procedure; a lathing procedure; or a combination thereof. The cannula 12 made from any desirable material such as, for example: a metallic material; a plastic material; or a combination thereof. In some examples, the cannula 12 may be made from a stainless steel material. In other instances, the cannula 12 may be made from an aluminum material. In yet other examples, the cannula 12 may be made from a detectable material such as, for example, a detectable alloy, a ferromagnetic alloy, a magnetically-detectable material, a magnetic resonance imaging (MRI) detectable material, a material that absorbs X-rays, or the like.

The cannula 12 may be defined in terms of ‘gauge size’ that takes into consideration skid/hide thickness of the subject S and/or a depth of injection of the subject S. The gauge size of the cannula 12 may be defined in a series of industry standard numbers in which, for example, the lower the number, the wider the diameter of the cannula. Furthermore, the series of industry standard numbers defining gauge size of the cannula 12 may be defined in a manner such that, for example, a higher gauge number indicates a smaller width of the cannula 12. In some instances, the industry standard gauge sizes of the cannula 12 may range from, for example: 14-Gauge; 16-Gauge; 18-Gauge; and 20-Gauge. Accordingly, in the range of exemplary industry standard numbers described above, a 14-Gauge cannula may be said to have a relatively largest diameter and highest strength (in terms of bendability/flexibility to a point where the cannula 12 could potentially break/fail) whereas a 20-Gauge cannula may be said to have a relatively smallest diameter and lowest strength (in terms of bendability/flexibility to a point where the cannula 12 could potentially break/fail).

A central axis A₁₂-A₁₂ extends through an axial center of the tube-shaped body 16 and along the length L₁₂ of the tube-shaped body 16. As will be described in the following disclosure at FIG. 59D and as seen at FIG. 2, a portion of the length L₁₂ of the tube-shaped body 16 (see, e.g., the sub-length portion L_{12c}) may bend, flex, or deviate from the central axis A₁₂-A₁₂ that extends through an axial center of the tube-shaped body 16. The sub-length portion L_{12c} of the length L₁₂ of the tube-shaped body 16 that may bend, flex, or deviate from the central axis A₁₂-A₁₂ extends generally along an axis A₁₂′-A₁₂′ that may be said to be not aligned with and deviate away from the central axis A₁₀-A₁₀ extending through the hypodermic interface assembly 10 when the cannula 12 is arranged as a component of the hypodermic interface assembly 10.

The tube-shaped body 16 is further defined by a proximal end surface 18 at the proximal end 16_{P} of the tube-shaped body 16 and a distal end surface 20 at distal end 16_{D} of the tube-shaped body 16. The tube-shaped body 16 is further defined by an outer surface 22 extending between the proximal end surface 18 and the distal end surface 20. The tube-shaped body 16 is further defined by an inner surface 24 extending between the proximal end surface 18 and the distal end surface 20. The inner surface 24 further defines a passage 26 extending through the tube-shaped body 16. The proximal end surface 18 defines a proximal opening 28 that is in fluid communication with the passage 26. The distal end surface 20 defines a distal opening 30 that is in fluid communication with the passage 26.

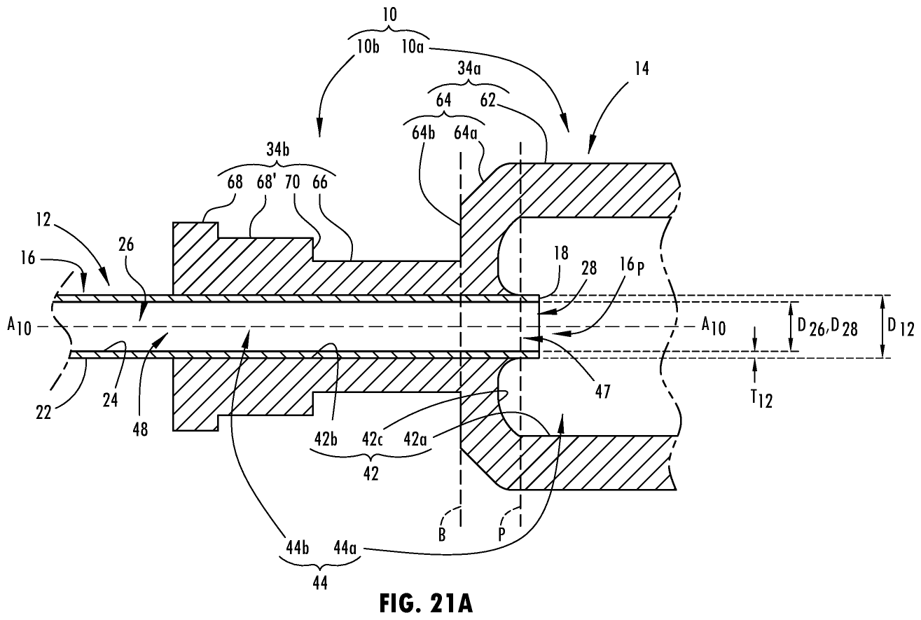

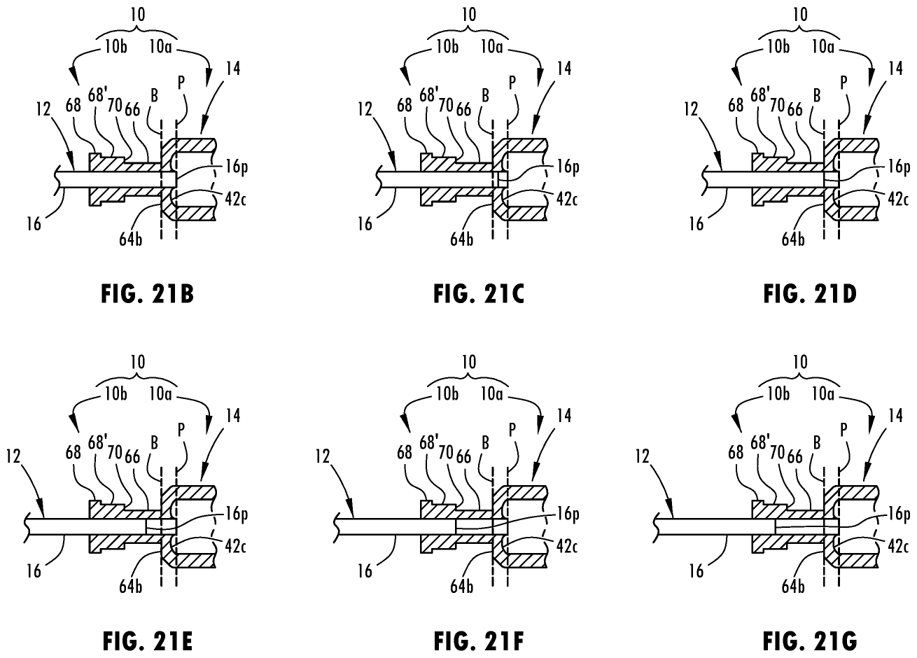

With reference to FIG. 21A, which illustrates an enlarged cross-section view of an exemplary hypodermic interface assembly 10, the body 16 of the cannula 12 is defined by a thickness T₁₂ extending between the outer surface 22 of the body 16 and the inner surface 24 of the body 16. The outer surface 22 further defines an outer diameter D₁₂ of the cannula 12 that is referenced from the central axis A₁₂-A₁₂, which may be coincident with respective central axes A₁₀-A₁₀ and A₁₄-A₁₄ of each of the hypodermic interface assembly 10 and the hub 14. The inner surface 24 further defines the passage 26 to have a passage diameter D₂₆. The passage 26 is in fluid communication with the proximal opening 28 and the distal opening 30 in order to permit: (1) passage of a fluid F (see, e.g., FIG. 59C) into the tube-shaped body 16 at the proximal opening 28; (2) through the passage 26 in a direction from the proximal end 16_{P} of the tube-shaped body 16 and towards the distal end 16_{D} of the tube-shaped body 16; and (3) out of the distal opening 30.

With reference to FIGS. 2 and 21A, the proximal end surface 18 extends from the outer surface 22 substantially perpendicularly, and, as such, defines the proximal end surface 18 to be blunted or non-sharpened. Furthermore, the proximal opening 28 formed by the proximal end surface 18 may define a substantially circular-shaped geometry that is defined by a proximal opening diameter D₂₈ that is substantially similar to the passage diameter D₂₆ of the passage 26.

With reference to FIG. 2, the distal end surface 20 extends from the outer surface 22 at a beveled angle θ₂₀, and, as such, the distal end surface 20 may be referred to as a beveled distal end surface that terminates at or defines a sharp piercing tip 32. The beveled distal end surface 20 may be defined by any desirable beveled angle θ₂₀ that forms, for example, a “standard bevel,” a “short bevel,” or a “true short bevel.” Because the beveled distal end surface 20 extends from the outer surface 22 at a beveled angle θ₂₀, the distal opening 30 may be defined by an oval-shaped geometry.

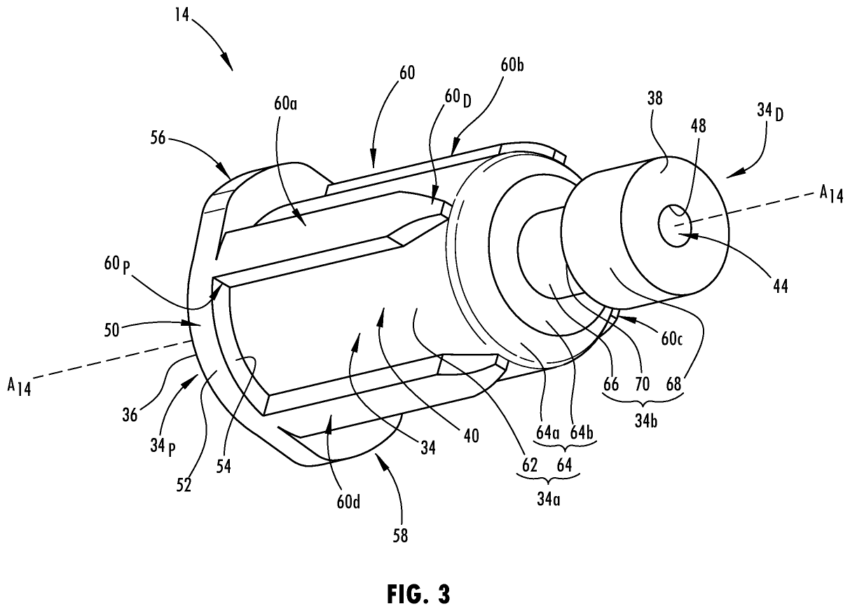

As seen at FIGS. 3-10, the hub 14 is defined by a substantially tube-shaped body 34 having a proximal end 34_{P} and a distal end 34D. The substantially tube-shaped body 34 is further defined by a neck portion 65 and a head portion 67, each of which generally circumscribes the central axis A₁₄-A₁₄ of the hub 14 (as seen at FIG. 7).

Even further, the hub 14 is defined by a length L₁₄ (see, e.g., FIG. 7) extending between the proximal end 34_{P} of the substantially tube-shaped body 34 and the distal end 34D of the substantially tube-shaped body 34. The length L₁₄ of the hub 14 is defined by a plurality of sub-lengths L₁₄ₐ (including sub-length portions L₁₄ₐ₁ and L₁₄ₐ₂), L_{14b} (including sub-length portions L_{14b1} and L_{14b2}), and L_{14c}, which will be further described in the following disclosure.

The hub 14 may be formed using any desirable manufacturing procedure such as, for example: a molding procedure; a casting procedure; a machining procedure; a lathing procedure; or a combination thereof. The hub 14 made from any desirable material such as, for example: a metallic material; a plastic material; or a combination thereof. In some examples, the hub 14 may be made from a stainless steel material. In other instances, the hub 14 may be made from an aluminum material, brass, steel, or alloys. In other examples, the hub 14 may be made from plastic materials including but not limited to polypropylene (PP), polyethylene terephthalate (PET), polyamides (e.g., nylon 6, nylon 6, 6, thermosetting plastics such as polyester resins, epoxy resins, acrylics), and the like. Furthermore, in some instances, the hub 14 may be finished with an anodization, a polishing, an electro-polishing, a coating, a paint or the like with, for example, a highly visible finish (e.g., a dye, a fluorescent coating, a phosphorescent coating, a bright gloss, matt color finish, or the like).

The substantially tube-shaped body 34 is further defined by a proximal end surface 36 at the proximal end 34_{P} of the substantially tube-shaped body 34 and a distal end surface 38 at distal end 34_{D} of the substantially tube-shaped body 34. The substantially tube-shaped body 34 is further defined by an outer surface 40 extending between the proximal end surface 36 and the distal end surface 38. The substantially tube-shaped body 34 is further defined by an inner surface 42 extending between the proximal end surface 36 and the distal end surface 38.

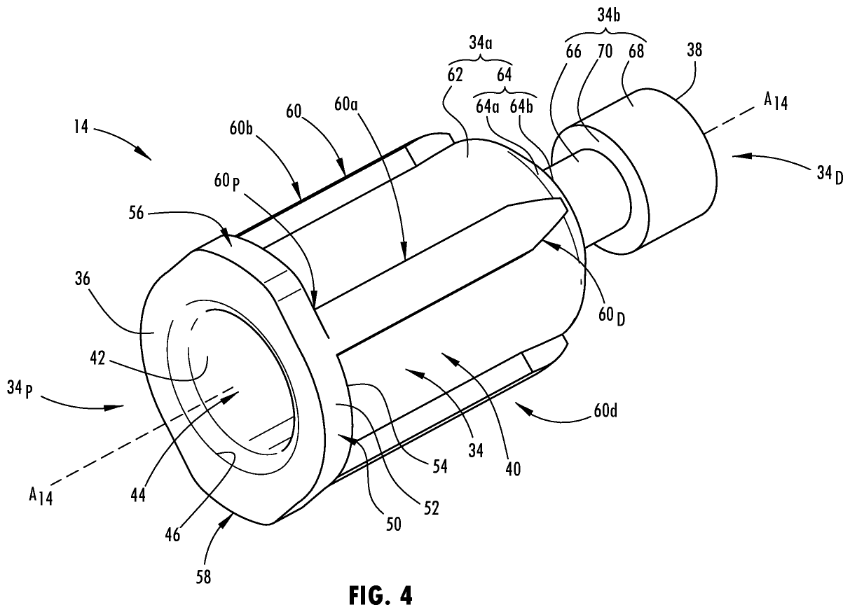

The inner surface 42 further defines a passage 44 extending through the substantially tube-shaped body 34. The proximal end surface 36 defines a proximal opening 46 (see, e.g., FIGS. 4, 7, and 10) that is in fluid communication with the passage 44. The distal end surface 38 defines a distal opening 48 (see, e.g., FIGS. 3, 5, 7, 8B, 9B) that is in fluid communication with the passage 44.

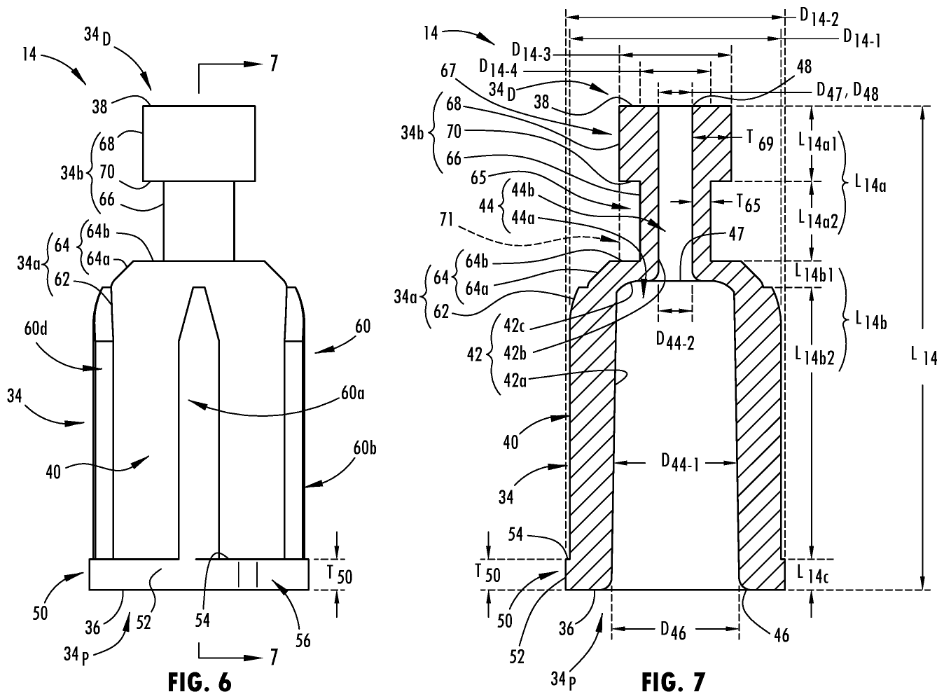

As seen at FIGS. 3-10, a ring portion 50 projects radially outwardly away from a central axis A₁₄-A₁₄ away from the outer surface 40 of the substantially tube-shaped body 34. The ring portion 50 may be alternatively referred to as a barrel-engaging portion that is configured to be connected to a barrel portion I_{B} of an injection gun I (see, e.g., FIG. 58). The barrel-engaging portion 50 is defined by an outer side surface 52 that extends between the proximal end surface 36 and a distal shoulder surface 54. The barrel-engaging portion 50 may be defined by a thickness T₅₀ (see, e.g., FIGS. 6 and 7) extending between the proximal end surface 36 and the distal shoulder surface 54. The barrel-engaging portion 50 may generally define a Luer lock.

The outer surface 40 of the substantially tube-shaped body 34 may define a substantially circular-shaped geometry that defines a first outer diameter D₁₄₋₁ (see, e.g., FIG. 7) of the hub 14. The outer side surface 52 of the barrel-engaging portion 50 may define a substantially circular-shaped geometry that defines a second outer diameter D₁₄₋₂ (see, e.g., FIG. 7) of the hub 14. The second outer diameter D₁₄₋₂ of the hub 14 is greater than the first outer diameter D₁₄₋₁ of the hub 14. The outer surface 40 of the substantially tube-shaped body 34 may further define another substantially circular-shaped geometry that further defines a third outer diameter D₁₄₋₃ (see, e.g., FIG. 7) of the hub 14. The outer surface 40 of the substantially tube-shaped body 34 may yet further define another substantially circular-shaped geometry that further defines a fourth outer diameter D₁₄₋₄ (see, e.g., FIG. 7) of the hub 14. The third outer diameter D₁₄₋₃ of the hub 14 and the fourth outer diameter D₁₄₋₄ of the hub 14 will be described in greater detail in the following disclosure.

As seen at FIGS. 3-5, 8A-8B, 9A-9B, and 10, the substantially circular-shaped geometry of the outer side surface 52 of the barrel-engaging portion 50 is interrupted by a first radially-outward projection or ear 56 and a second radially-outward projection or ear 58 that extend beyond the second outer diameter D₁₄₋₂ of the hub 14. The first radially-outward projection or ear 56 may be arranged opposite of or offset approximately 180° from the second radially-outward projection or ear 58.

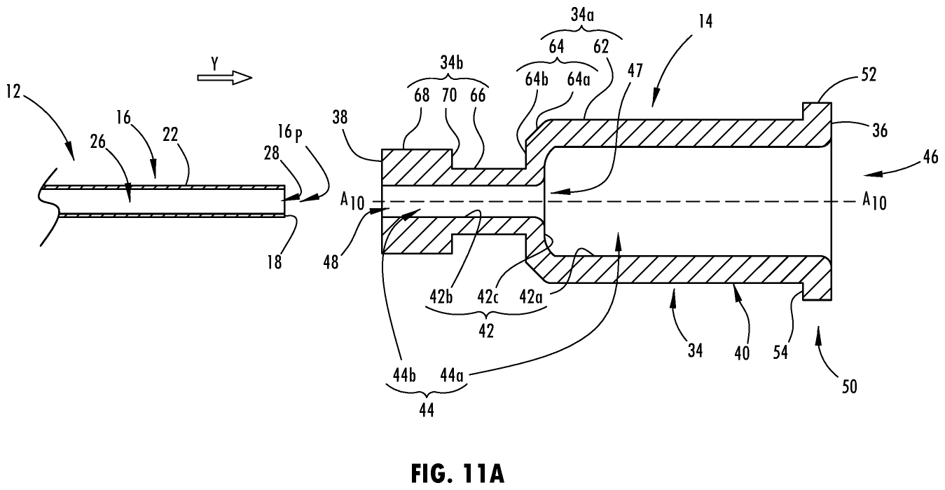

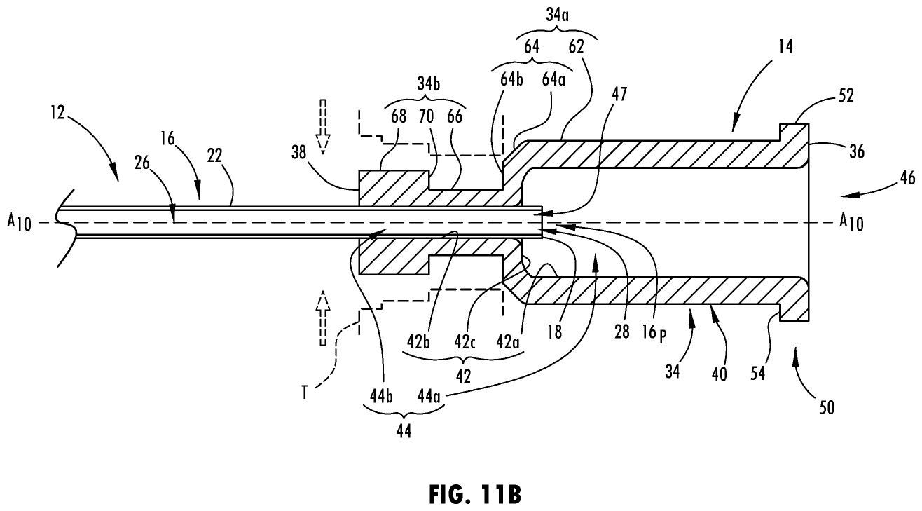

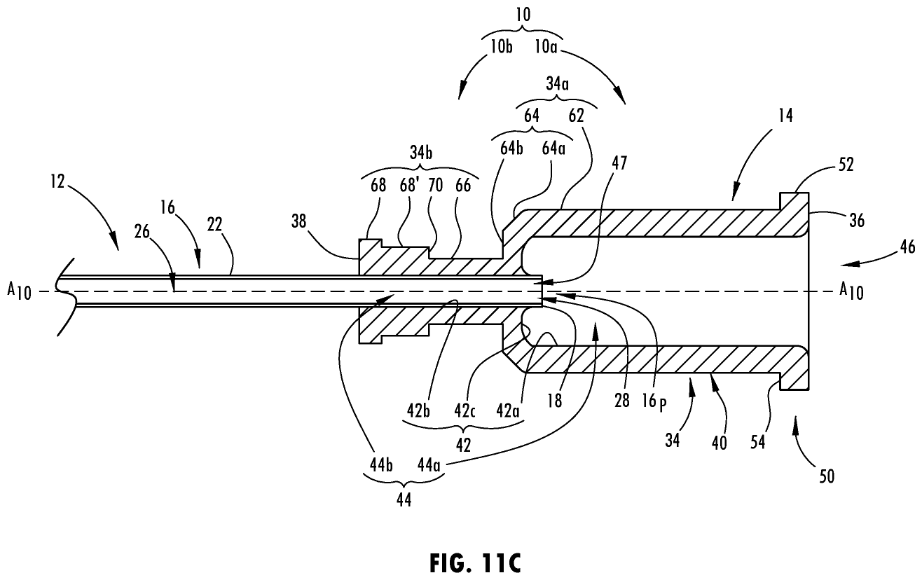

As seen at FIG. 7, the inner surface 42 of the substantially tube-shaped body 34 includes a first inner surface portion 42a, a second inner surface portion 42b, and a third inner surface portion 42c. Each of the first inner surface portion 42a and the second inner surface portion 42b generally circumscribe the central axis A₁₄-A₁₄ of the hub 14. The third inner surface portion 42c connects the first inner surface portion 42a to the second inner surface portion 42b; furthermore, the third inner surface portion 42c may be substantially orthogonal to the central axis A₁₄-A₁₄ of the hub 14. The third inner surface portion 42c may be substantially perpendicular with respect to each of the first inner surface portion 42a and the second inner surface portion 42b; in some implementations, the transition of each of the first inner surface portion 42a and the second inner surface portion 42b to the third inner surface portion 42c may be defines by a curved or arcuate segment. As will be seen in the following disclosure at FIGS. 11B-11C, after material deformation of at least a portion of, for example, the second portion 34b of the substantially tube-shaped body 34 of the hub 14 (e.g., by crimping a portion of, for example, the second portion 34b of the substantially tube-shaped body 34 of the hub 14 after the cannula 12 is interfaced with the hub 14 as seen as FIG. 11B), the curved or arcuate segment joining the second inner surface portion 42b to the third inner surface portion 42c may change in shape as a result of the material shifting or “flowing”, and, as such, a portion of the third inner surface portion 42c that extends from the second inner surface portion 42b may define a frustoconical surface portion (see, e.g., FIG. 11C) surrounding the cannula 12.

The first inner surface portion 42a of the inner surface defines a first passage portion 44a of the passage 44. The second inner surface portion 42b defines a second passage portion 44b of the passage 44.

The first passage portion 44a defines a first passage diameter D₄₄₋₁ (see, e.g., FIG. 7) of the passage 44. The second passage portion 44b defines a second passage diameter D₄₄₋₂ (see, e.g., FIG. 7) of the passage 44. The first passage diameter D₄₄₋₁ is greater than the second passage diameter D₄₄₋₂. The second passage diameter D₄₄₋₂ is approximately equal to but slightly greater than the outer diameter D₁₂ of the cannula 12.

The first passage portion 44a of the passage 44 is in fluid communication with the proximal opening 46, and the second passage portion 44b of the passage 44 is in fluid communication with the distal opening 48; furthermore, the first passage portion 44a is in fluid communication with the second passage portion 44b by way of an intermediate opening 47. Accordingly, the passage 44 permits: (1) passage of a fluid F (see, e.g., FIG. 59C) into the substantially tube-shaped body 34 at the proximal opening 46; (2) through the first passage portion 44a of the passage 44 in a direction from the proximal end 34_{P} of the substantially tube-shaped body 34 and towards the intermediate opening 47 defined by the third inner surface portion 42c; (3) through the intermediate opening 47 that defines a proximal opening of the second passage portion 44b of the passage 44; (4) through the second passage portion 44b of the passage 44 in a direction from the intermediate opening 47 and towards the distal end 34D of the substantially tube-shaped body 34; and (5) out of the distal opening 48.

The proximal opening 46 formed by the proximal end surface 36 may define a substantially circular-shaped geometry that is defined by a proximal opening diameter D₄₆ (see, e.g., FIG. 7) that is substantially similar to the first passage diameter D₄₄₋₁ of the first passage portion 44a. The intermediate opening 47 formed by the third inner surface portion 42c of the inner surface 42 of the substantially tube-shaped body 34 may define a substantially circular-shaped geometry that is defined by an intermediate opening diameter D₄₇ (see, e.g., FIG. 7) that is substantially equal to the second passage diameter D₄₄₋₂. The distal opening 48 formed by the distal end surface 38 may define a substantially circular-shaped geometry that is defined by a distal opening diameter D₄₈ (see, e.g., FIG. 7) that is substantially similar to the second passage diameter D₄₄₋₂. Although some of the dimensions/diameters/geometries are described above to be substantially similar or the same, the view of the hub 14 in the Figures (e.g., at FIG. 7) are exemplary and are not to scale. In some instances, the first passage portion 44a may be formed to include a draft angle (e.g., a 1° draft angle) that, for example, may assist in the removal of the hub 14 from tooling when the hub 14 is formed; accordingly, the first passage diameter D₄₄₋₁ of the first passage portion 44a may progressively decrease in diameter as the first passage diameter D₄₄₋₁ of the first passage portion 44a extends in a direction from the proximal end surface 36 of the hub 14 toward the distal end surface 38 of the hub 14.

Referring to FIGS. 3-6, 8A, 8B, 9A, and 9B, one or more ribs 60 may project radially outwardly away from a central axis A₁₄-A₁₄ away from an outer body surface portion 62 defined by the outer surface 40 of the substantially tube-shaped body 34. The one or more ribs 60 may include, for example, a first rib 60a, a second rib 60b, a third rib 60c, and a fourth rib 60d.

The one or more ribs 60 may increase the structural integrity of the substantially tube-shaped body 34 of the hub 14. In some configurations, the one or more ribs 60 may arise from mold relief features during the manufacturing process of the substantially tube-shaped body 34 of the hub 14. Furthermore, the one or more ribs 60 may be configured to engage packaging (not shown). Engagement of the one or more ribs 60 with the packaging may assist in containing the cannula 12 and the hub 14 shipping and/or assist in engagement/disengagement of the hub 14 with/from the injection gun I.

Each rib 60a, 60b, 60c, 60d of the one or more ribs 60 includes a distal end 60_{D} and a proximal end 60_{P}. The proximal end 60_{P} of each rib 60a, 60b, 60c, 60d of the one or more ribs 60 extends from the distal shoulder surface 54 of the barrel-engaging portion 50. The distal end 60D of each rib 60a, 60b, 60c, 60d of the one or more ribs 60 extends in a direction toward the distal end surface 38 of the substantially tube-shaped body 34 and terminates at, before, or near an outer shoulder surface portion 64 (see, e.g., FIGS. 3-9) defined by the outer surface 40 of the substantially tube-shaped body 34. Each rib 60a, 60b, 60c, 60d of the one or more ribs 60 may define a substantially rectangular body that terminates with a substantially triangular body portion defined by the distal end 60D of each rib 60a, 60b, 60c, 60d of the one or more ribs 60.

The outer shoulder surface portion 64 extends from a distal-most end of the outer body surface portion 62 of the outer surface 40 of the substantially tube-shaped body 34. In some configurations, the outer shoulder surface portion 64 may define a dome-shaped or curved outer shoulder surface portion 64a that peaks or terminates at a substantially flat outer shoulder surface portion 64b.

Referring to FIGS. 3-7, 8A, and 9A, the outer surface 40 of the substantially tube-shaped body 34 may further define: (1) an outer neck surface portion 66 (extending around the neck portion 65 of substantially tube-shaped body 34); (2) an outer head surface portion 68 (extending around the head portion 67 of substantially tube-shaped body 34); and (3) an intermediate surface portion 70. Each of the outer neck surface portion 66 and the outer head surface portion 68 generally circumscribe the central axis A₁₄-A₁₄ of the hub 14. The intermediate surface portion 70 extends between and connects the outer neck surface portion 66 to the outer head surface portion 68; furthermore, the intermediate surface portion 70 may be substantially orthogonal to the central axis A₁₄-A₁₄ of the hub 14.

The outer neck surface portion 66 extends between and connects the outer shoulder surface portion 64 to the intermediate surface portion 70. The outer head surface portion 68 extends between and connects the intermediate surface portion 70 to the distal end surface 38 of the substantially tube-shaped body 34. As seen at FIG. 7, the outer head surface portion 68 may generally define the outer surface of the head portion 67, which head portion 67 extends proximally away from the distal end surface 38 of substantially tube-shaped body 34. Further, the outer neck surface portion 66 may generally define the outer surface of the neck portion 65, which neck portion 65 extends proximally away from the intermediate surface portion 70.

The neck portion 65 is defined by a thickness T₆₅ extending between the second inner surface portion 42b of the inner surface 42 (of the substantially tube-shaped body 34) and the outer neck surface portion 66 of the outer surface 40. The thickness T₆₅ may be uniform and remain substantially constant throughout its length, i.e., the length L₁₄ₐ₂ of the outer neck surface portion 66. However, in other exemplary embodiments described throughout the following disclosure, the thickness T₆₅ may not be uniform and may be non-constant throughout its length. For example: (1) as shown at FIG. 26, where a substantially “conical” neck portion 165 is defined by an outer neck surface portion 166, a thickness T₁₆₅ decreases along a length L₁₁₄ₐ₂ from a maximum thickness T₁₆₅ at a distal end of the neck portion 165 to a minimum thickness T₁₆₅ at a proximal end of the neck portion 165; (2) as shown at FIG. 35, where a substantially “narrow V-shaped” neck portion is defined by a V-shaped notch or groove 265, a thickness T₂₆₅ decreases from a maximum thickness T₂₆₅ at a distal end of the V-shaped notch or groove 265 to a minimum thickness T₂₆₅ at the “valley” of the V-shaped notch or groove 265 and then increases back to maximum thickness T₂₆₅ at a proximal end of the V-shaped notch or groove 265 as the V-shaped notch or groove 265 extends along a length L₂₁₄ₐ₂ of an outer neck surface portion 266 of an outer surface 240 of a substantially tube-shaped body 234 of a hub 214; (3) as shown at FIG. 44, where a substantially “wider V-shaped” neck portion is defined V-shaped notch or groove 365, a thickness T₃₆₅ decreases from a maximum thickness T₃₆₅ at a distal end of the V-shaped notch or groove 365 to a minimum thickness T₃₆₅ at the “valley” of the V-shaped notch or groove 365 and then increases back to maximum thickness T₃₆₅ at a proximal end V-shaped notch or groove 365 as the V-shaped notch or groove 365 extends along a length L₃₁₄ₐ₂ of an outer neck surface portion 366 of an outer surface 340 of a substantially tube-shaped body 334 of a hub 314; and (4) as shown at FIG. 53, where a substantially “dome-and-V-shaped-groove” neck portion is defined by a combination of a dome-shaped surface portion 464a and a V-shaped notch or groove 465, a thickness T₄₆₅ (according to a direction extending from the distal end toward the proximal end of a hub 414) firstly increases along a portion of the length L₄₁₄ₐ₂ defined by the dome-shaped surface portion 464a and then decreases and increases according to the V-shaped notch or groove 465. The “valley” of any of the V-shaped notch or grooves 265, 365, 465 is not limited according to the exemplary implementations seen at FIGS. 35, 44, and 53. Accordingly, if desired, the valley of any of the V-shaped notch or grooves 265, 365, 465 may extend into the material defining the hub 214, 314, 414 such that any of the thicknesses T₂₆₅, T₃₆₅, T₄₆₅ could be approximately equal to a dimension slightly greater than zero millimeters, inches, or the like.

The head portion 67 is defined by a thickness T₆₉ extending between the second inner surface portion 42b of the inner surface 42 (of the substantially tube-shaped body 34) and the outer head surface portion 68 of the outer surface 40. The thickness T₆₉ may be uniform and remain substantially constant throughout its length, i.e., the length L₁₄ₐ₁ of the outer head surface portion 68. In other exemplary embodiments described throughout the following disclosure, other exemplary hubs 114, 214, 314, and 414 may also define necks having a uniform and constant thicknesses T₁₆₅, T₂₆₅, T₃₆₅, and T₄₆₅ (see, e.g., FIGS. 26, 35, 44, and 53). However, other exemplary implementations of any of the thicknesses T₆₅, T₁₆₅, T₂₆₅, T₃₆₅, and T₄₆₅ may not be uniform and may be non-constant throughout its length.

As seen at FIG. 7, the outer head surface portion 68 of the outer surface 40 of the substantially tube-shaped body 34 defines the third outer diameter D₁₄₋₃ of the hub 14. The outer neck surface portion 66 of the outer surface 40 of the substantially tube-shaped body 34 defines the fourth outer diameter D₁₄₋₄ of the hub 14. As seen at FIG. 7, the third outer diameter D₁₄₋₃ is greater than the fourth outer diameter D₁₄₋₄, and the second outer diameter D₁₄₋₂ is greater than the third outer diameter D₁₄₋₃.

As seen at FIGS. 3-7, 8A, and 9A, the outer body surface portion 62 and the outer shoulder surface portion 64 of the outer surface 40 define a first portion 34a of the substantially tube-shaped body 34 of the hub 14. The outer neck surface portion 66, the outer head surface portion 68, and the intermediate surface portion 70 of the outer surface 40 define a second portion 34b of the substantially tube-shaped body 34 of the hub 14.

With reference to FIG. 7, the sub-length portion L₁₄ₐ₁ defines the length of the outer head surface portion 68 of the outer surface 40 of the substantially tube-shaped body 34 and extends between: (1) the intermediate surface portion 70 of the outer surface 40 of the substantially tube-shaped body 34; and (2) the distal end surface 38 of the substantially tube-shaped body 34. The sub-length portion L₁₄ₐ₂ defines the length of the outer neck surface portion 66 of the outer surface 40 of the substantially tube-shaped body 34 and extends between: (1) the intermediate surface portion 70 of the outer surface 40 of the substantially tube-shaped body 34; and (2) the substantially flat outer shoulder surface portion 64b of the outer shoulder surface portion 64 of the outer surface 40 of the substantially tube-shaped body 34. The sub-length portion L₁₄ₐ₂ may be any desirable dimension or in a ratio. In some instances, the length of the sub-length portion L₁₄ₐ₂ may be in a range between approximately 0.25 and 4 times the fourth outer diameter D₁₄₋₄. In other configurations, the length of the sub-length portion L₁₄ₐ₂ may be in a range between approximately 0.50 and 3 times the fourth outer diameter D₁₄₋₄. In yet other configurations, length of the sub-length portion L₁₄ₐ₂ may be in a range between approximately 1 and 2 times the fourth outer diameter D₁₄₋₄ Any of the exemplary the sub-length portion L₁₄ₐ₂ ranges described above may be define a sufficient length that permits a portion of the outer neck surface portion 66 to break, tear, rip, or otherwise fail before the intermediate surface portion 70 touches or is arranged adjacent the outer shoulder surface portion 64; as such, after the cannula 12 pierces the subject S and the cannula 12 is subjected to one or more radial forces X_{R} relative to the central axis A₁₀-A₁₀, the second portion 10b of the hypodermic interface assembly 10 may predictably and controllably separate from the first portion 10a of the hypodermic as seen at FIG. 11B interface assembly 10.

Further, the neck thickness T₆₅ of the neck 65 may be defined by a sufficient thickness that permits the second portion 10b of the hypodermic interface assembly 10 to predictably and controllably separate from the first portion 10a of the hypodermic interface assembly 10. The thickness T₆₅ of the neck 65 may be any desirable dimension or in a ratio. In some instances, the thickness T₆₅ may be in a range between approximately 0.10 and 0.95 times the thickness T₆₉ of the head 67. In other configurations, the thickness T₆₅ may be in a range between approximately 0.20 and 0.80 times the thickness T₆₉ of the head 67. In yet other configurations, the thickness T₆₅ may be in a range between approximately 0.33 and 0.66 times the thickness T₆₉ of the head 67.

While not wishing to be bound by any theory, after the cannula 12 pierces the subject S and is subjected to one or more radial forces X_{R} relative to the central axis A₁₀-A₁₀, a portion of the neck 65 of substantially tube-shaped body 34 may break, tear, rip, or otherwise fail because of deformations in the neck 65, as follows: (1) a first portion of the neck 65 of substantially tube-shaped body 34 may begin to crumple or collapse and (2) a second portion of the neck 65 of substantially tube-shaped body 34 may begin to fold in a direction toward the first portion of the neck 65, e.g., the first portion of the neck 65 and the second portion of neck 65 are opposite portions of the neck 65 (180° apart). These deformations weaken the neck 65, and eventually the neck 65 becomes weak enough to break, tear, rip, or otherwise fail.

In one embodiment, the length of the sub-length portion L₁₄ₐ₁ of sub-length L₁₄ₐ is greater than the length of the sub-length portion L₁₄ₐ₂ of sub-length L₁₄ₐ. For example, the length of the sub-length portion L₁₄ₐ₁ may be about two times (2×) greater than, about three times (3×) greater than, or about four times (4×) greater than, the length of the sub-length portion L₁₄ₐ₂. Further, the length of the sub-length portion L₁₄ₐ₁ of sub-length L₁₄ₐ may be of sufficient length to allow for punching, crimping, swaging or materially deforming a portion or all of the outer head surface portion 68 of the outer surface 40 of the substantially tube-shaped body 34 (see, e.g., reference numeral 68′). Collectively the sub-length portion L₁₄ₐ₁ and the sub-length portion L₁₄ₐ₂ define the sub-length L₁₄ₐ, which defines the length of the second portion 34b of the substantially tube-shaped body 34 of the hub 14.

The sub-length portion L_{14b1} defines the length (or thickness) of the outer shoulder surface portion 64 of the outer surface 40 of the substantially tube-shaped body 34 and extends between the: (1) the substantially flat outer shoulder surface portion 64b of the outer shoulder surface portion 64 of the outer surface 40 of the substantially tube-shaped body 34; and (2) the distal-most end of the outer body surface portion 62 of the outer surface 40 of the substantially tube-shaped body 34. The sub-length portion L_{14b2} defines the length of the outer body surface portion 62 of the outer surface 40 of the substantially tube-shaped body 34 and extends between: (1) the distal-most end of the outer body surface portion 62 of the outer surface 40 of the substantially tube-shaped body 34; and (2) the distal shoulder surface 54 of the barrel-engaging portion 50. Collectively the sub-length portion L_{14b1} and the sub-length portion L_{14b2} define the sub-length L_{14b}, which, along with the sub-length L_{14c} (that defines a thickness of the barrel-engaging portion 50) defines the length of the first portion 34a of the substantially tube-shaped body 34 of the hub 14.

The first portion 34a of the substantially tube-shaped body 34 of the hub 14 may define at least a portion of or all of the first portion 10a of the hypodermic interface assembly 10 that is configured to remain attached to the injection gun I after the cannula 12 is subjected to one or more radial forces X_{R} relative to the central axis A₁₀-A₁₀ extending through the hypodermic interface assembly 10. The second portion 34b of the substantially tube-shaped body 34 of the hub 14 may define a first component of the second portion 10b of the hypodermic interface assembly 10 (with a second component of the second portion 10b of the hypodermic interface assembly 10 being the cannula 12) that is configured to predictably and controllably separate from the first portion 10a of the hypodermic interface assembly 10 after the cannula 12 is subjected to the one or more radial forces X_{R} relative to the central axis A₁₀-A₁₀ extending through the hypodermic interface assembly 10. Accordingly, as will be explained in the following disclosure, upon predictably separating the second portion 34b of the substantially tube-shaped body 34 of the hub 14 from the first portion 34a of the substantially tube-shaped body 34 of the hub 14, a user may easily locate and grasp from the flesh of the animalia S (see, e.g., FIG. 59F) the second portion 34b of the substantially tube-shaped body 34 of the hub 14 in order to remove the cannula 12 (see, e.g., FIG. 59G), which defines the second component of the second portion 10b of the hypodermic interface assembly 10. As such, should the one or more radial forces X_{R} relative to the central axis A₁₀-A₁₀ extending through the hypodermic interface assembly 10 be imparted to the cannula 12 during the course of utilizing the hypodermic interface assembly 10, the cannula 12 would not be lost within the flesh of the animalia S.

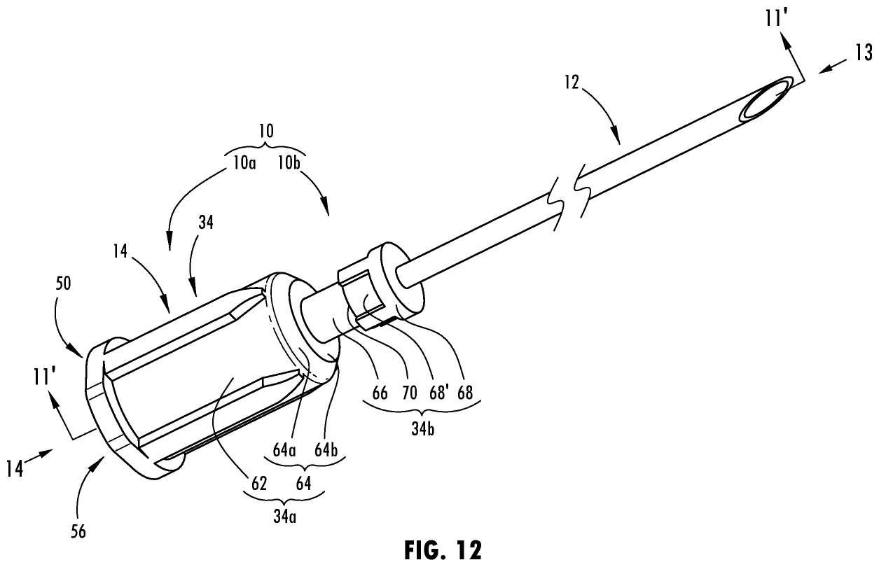

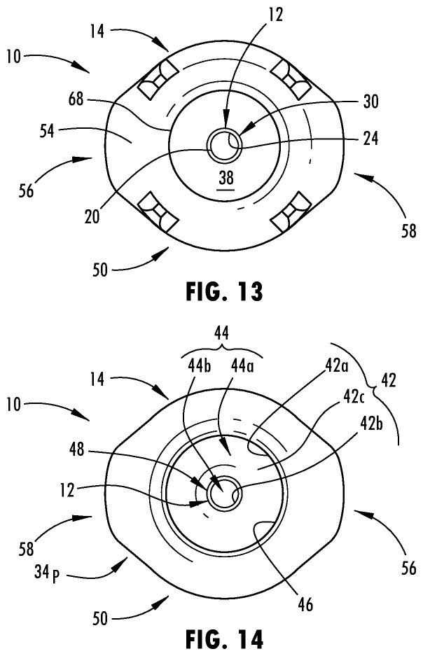

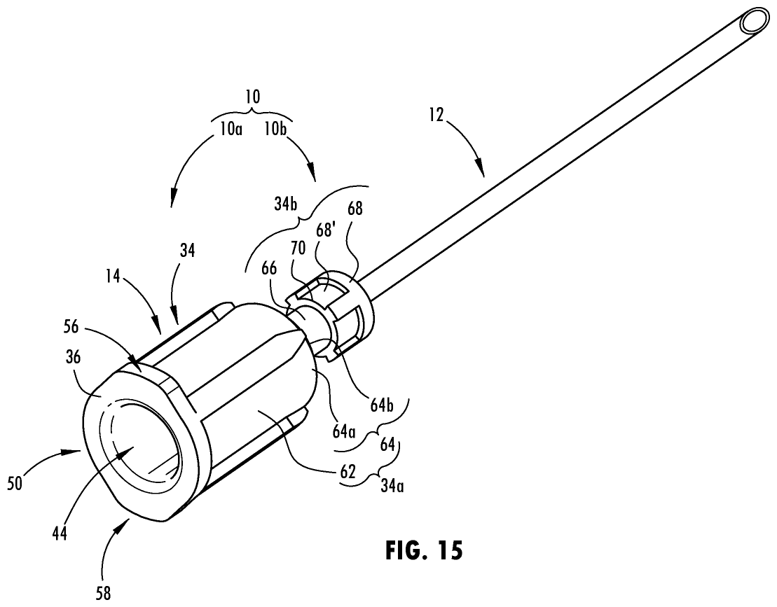

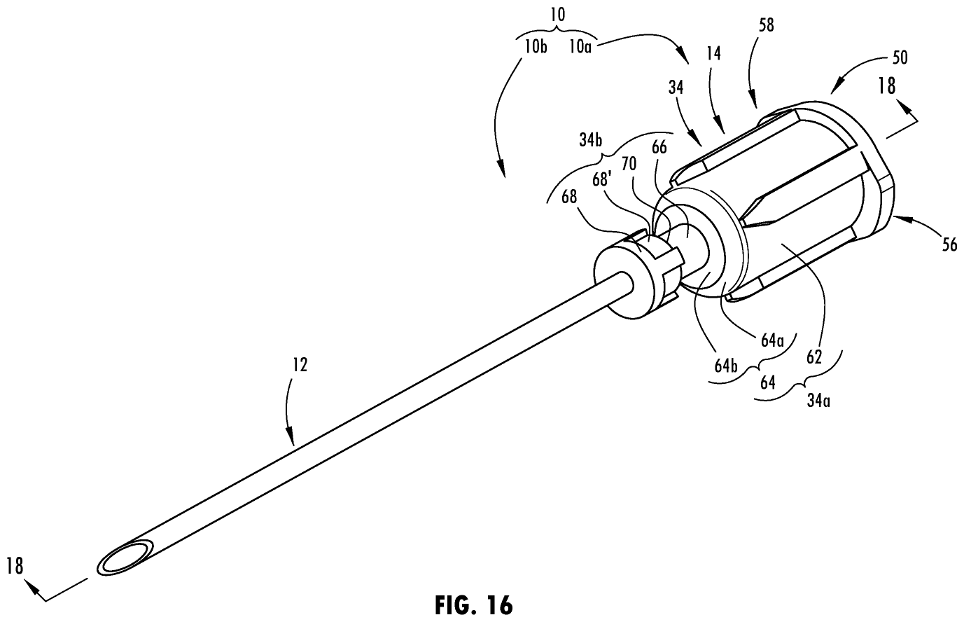

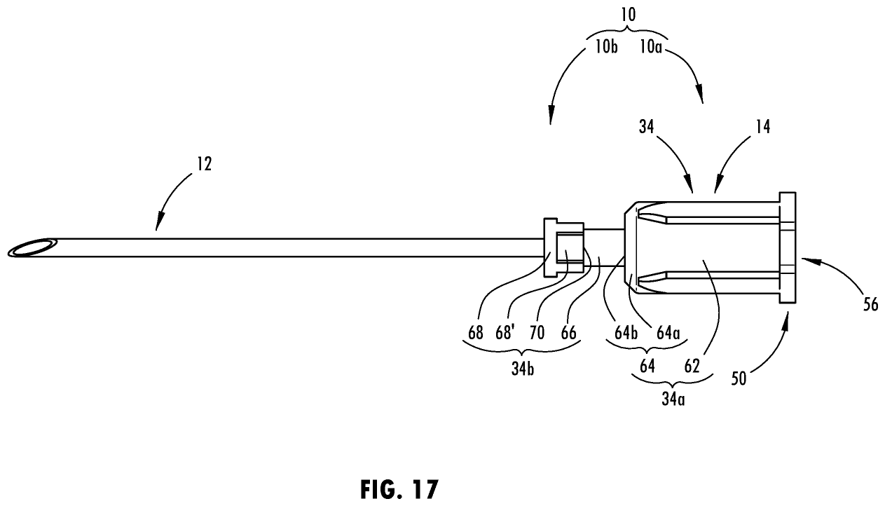

Referring to FIGS. 11A-11C, a method for assembling the hypodermic interface assembly 10, which is shown in assembled form at FIGS. 12-18B and 21A, is described. Firstly, at FIG. 11A, the components (i.e., the cannula 12 and the hub 14 of the hypodermic interface assembly 10 are axially aligned about a central axis A₁₀-A₁₀ (see also FIG. 1). The central axis A₁₀-A₁₀ corresponds to, for example, the central axes A₁₂-A₁₂, A₁₄-A₁₄ of each of the cannula 12 and the hub 14.

As will be described in the following disclosure, the cannula 12 is mechanically joined to any portion of the hub 14 as a result of, for example, material deformation of at least a portion of, for example, the second portion 34b of the substantially tube-shaped body 34 of the hub 14 (e.g., by crimping a portion of, for example, the second portion 34b of the substantially tube-shaped body 34 of the hub 14 after the cannula 12 is interfaced with the hub 14 as seen as FIG. 11B). Although the hypodermic interface assembly 10 is formed by a mechanical connection, the cannula 12 may alternatively or additionally be joined to any portion of the hub 14, such as, for example, the second portion 34b of the substantially tube-shaped body 34 of the hub 14 with, for example, an adhesive (not shown), such as, for example: an acrylic adhesive, a cyanoacrylate adhesive, a ultra-violet (UV) curable adhesive, or the like.

As seen at FIG. 11A, a portion of the cannula 12 including the proximal end surface 18 at the proximal end 16_{P} of the tube-shaped body 16 is shown arranged near the distal opening 48 (that is in fluid communication with the second passage portion 44b of the passage 44 of the hub 14) formed by the distal end surface 38 of the hub 14. The central axis A₁₂-A₁₂ (see, e.g., FIG. 2) of the cannula 12 is axially aligned with the central axis A₁₄-A₁₄ of the hub 14. The central axes A₁₂-A₁₂ and A₁₄-A₁₄ of each of the cannula 12 and the hub 14 correspond to the central axis A₁₀-A₁₀ (see FIG. 1) of the hypodermic interface assembly 10.

As described above, the outer surface 22 of the tube-shaped body 16 of the cannula 12 defines an outer diameter D₁₂ of the cannula 12, and the second passage diameter D₄₄₋₂ that defines the second passage portion 44b of the passage 44 is approximately equal to but slightly greater than the outer diameter D₁₂ of the cannula 12 so that at least a portion of the second passage portion 44b of the passage 44 defined by the second portion 34b of the substantially tube-shaped body 34 of the hub 14 is configured to receive the cannula 12. Then, as seen at FIGS. 11B-11C, the proximal end 16_{P} of the tube-shaped body 16 of the cannula 12 is inserted (according to the direction of the arrow Y as seen at FIG. 11A) through the distal opening 48 formed by the distal end surface 38 of the hub 14 and then disposed within at least a portion of the second passage portion 44b of the passage 44 of the hub 14. In some configurations as seen at, for example, FIGS. 11C and 21A, the cannula 12 may be arranged relative the hub 14 such that the proximal end 16_{P} of the tube-shaped body 16 of the cannula 12 is arranged beyond the third inner surface portion 42c (see, e.g., dashed line P at FIG. 21A) of the inner surface 42 of the substantially tube-shaped body 34 such that a portion of the cannula 12 is arranged within an entirely occupies the second passage portion 44b of the passage 44 of the hub 14 while also being partially disposed within the first passage portion 44a of the passage 44 of the hub 14.

Thereafter, as seen at FIG. 11B, the cannula 12 is arranged within the passage 44 of the hub 14 in order to subsequently, for example, mechanically join the cannula 12 to the hub 14 by, for example, arranging the second portion 34b of the substantially tube-shaped body 34 of the hub 14 within, for example, a crimping tool T. The crimping tool T may punch, crimp, swage or materially deform, for example, all or a portion of the head 67 of the hub 14 in order to mechanically connect a portion of the second inner surface portion 42b of the inner surface 42 of the substantially tube-shaped body 34 of the hub 14 to a portion of the length (see, e.g., sub-length L₁₂ₐ₁ at FIG. 2) of the outer surface 22 of the tube-shaped body 16 of the cannula 12 in a friction-fit relationship, an interference-fit relationship, or a mechanically-coupled relationship.

Accordingly, as seen at FIG. 11C, the cannula 12 may be mechanically joined to the hub 14 as a result of the material deformation of the portion of the hub 14 by the crimping tool T. After the cannula 12 is joined to the hub 14, a ring (not shown) that is formed from a highly visible material may be fitted to one or both of the cannula 12 and the hub 14 in order to assist in visually locating the second portion 10b of the hypodermic interface assembly 10 after the second portion 10b of the hypodermic interface assembly 10 is predictably and controllably separated from the first portion 10a of the hypodermic interface assembly 10.

In some configurations, as seen at FIG. 11C and FIGS. 12 and 15-18B, the crimping tool T may punch, crimp, swage or materially deform, for example, a portion of the outer surface 40 of the substantially tube-shaped body 34. In some implementations, for example, the crumping tool T may punch, crimp, swage or materially deform (see, e.g., reference numeral 68′ along the sub-length portion L₁₄ₐ₁ of sub-length L₁₄ₐ), for example, a portion or all of the outer head surface portion 68 of the outer surface 40 of the substantially tube-shaped body 34. Accordingly, in such implementations, the outer head surface portion 68 of the outer surface 40 of the substantially tube-shaped body 34 may define crimping pockets (see, e.g., reference numeral 68′) that infer material deformation of the outer head surface portion 68 of the outer surface 40 of the substantially tube-shaped body 34, and, as a result, distinguishes a “deformed” hub 14 that is mechanically connected to the cannula 12 from a virgin or “non-deformed” hub 14 (see, e.g., FIGS. 3-10) having an outer head surface portion 68 of the outer surface 40 of the substantially tube-shaped body 34 that does not define crimping pockets 68′.

As described above at FIGS. 11A-11C, a portion of the length (see, e.g., sub-length L₁₂ₐ₁ as seen at FIG. 2) of the outer surface 22 of the tube-shaped body 16 of the cannula 12 is disposed within the second passage portion 44b of the passage 44 of the hub 14 and may be mechanically secured (see, e.g., FIG. 11C) to at least a portion of the second inner surface portion 42b of the inner surface 42 of the substantially tube-shaped body 34 of the hub 14, which may extend along a sub-length of the hub 14 defined by the length L₁₄ₐ₁ (see, e.g., FIG. 7) of the outer head surface portion 68 of the outer surface 40 of the substantially tube-shaped body 34 of the hub 14. Furthermore, with reference to FIGS. 11C and 21A, a portion of the length (see, e.g., sub-length L₁₂ₐ₂ at FIG. 2) of the outer surface 22 of the tube-shaped body 16 of the cannula 12 may be disposed within the second passage portion 44b of the passage 44 of the hub 14, which may extend along a sub-length of the hub 14 defined by the length L₁₄ₐ₂ of the outer neck surface portion 66 of the outer surface 40 of the substantially tube-shaped body 34 of the hub 14. This portion L₁₄ₐ₂ of the length L₁₄ of the hub 14 may, in some implementations, not be materially deformed by the crimping tool T, and, as such the sub-length L₁₂ₐ₂ of the cannula 12 may not be mechanically coupled to the hub 14 along the length L₁₄ₐ₂ of the outer neck surface portion 66 of the outer surface 40 of the substantially tube-shaped body 34 of the hub 14 since the hub 14 may not be materially deformed along the length L₁₄ₐ₂ of the outer neck surface portion 66 of the outer surface 40 of the substantially tube-shaped body 34 of the hub 14. The sub-length L₁₂ₐ₁ and the sub-length L₁₂ₐ₂ define the length portion L₁₂ₐ of the cannula 12.

Furthermore, as seen at FIGS. 11C and 21A, a portion of the length (see, e.g., length portion L_{12b}) of the outer surface 22 of the tube-shaped body 16 of the cannula 12 may be disposed within the first passage portion 44a of the passage 44 of the hub 14, which may extend along a sub-length of the hub 14 defined by a portion of the length L_{14b2} of the outer body surface portion 62 of the outer surface 40 of the substantially tube-shaped body 34. With reference to FIGS. 2, 11C, and 21A a remainder/length portion (see, e.g., length portion L_{12c}) of the outer surface 22 of the tube-shaped body 16 of the cannula 12 may extend beyond the distal end surface 38 of the hub 14 and is not contained within the passage 44 of the hub 14.

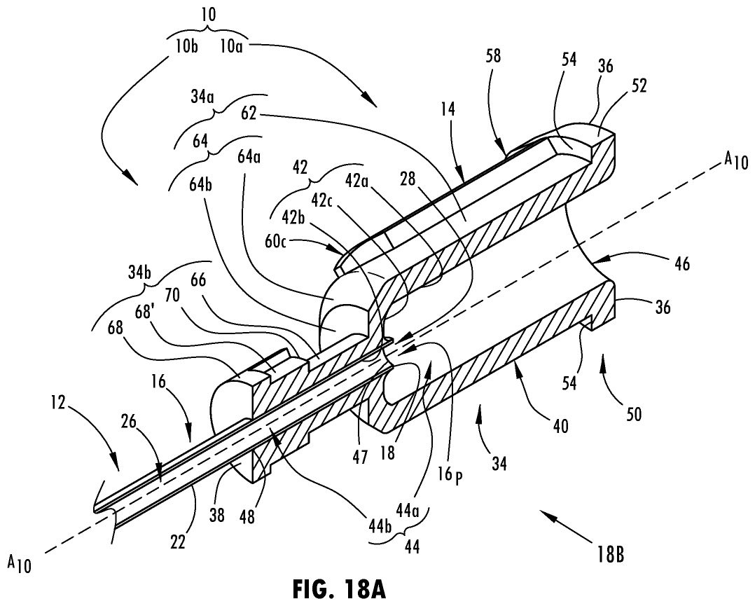

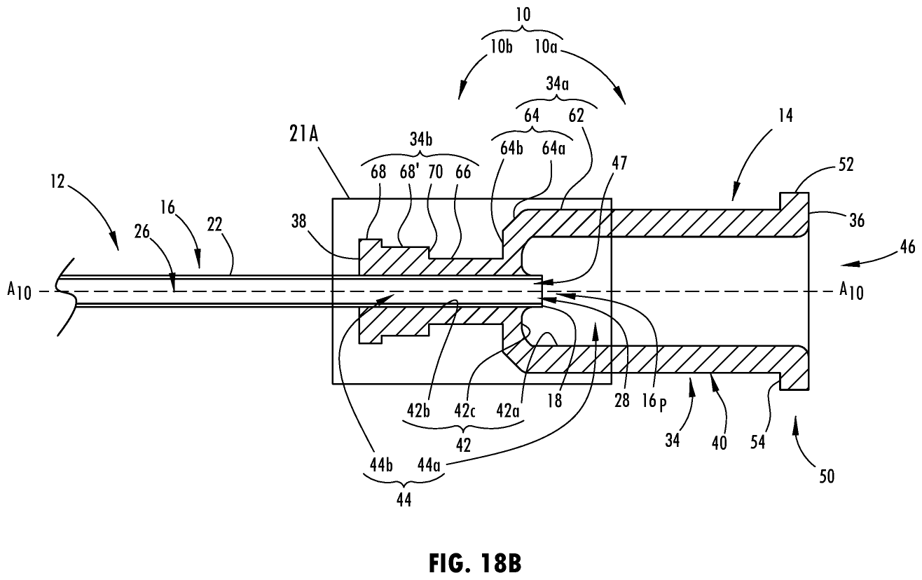

After the cannula 12 is joined to the hub 14, the 10 hypodermic interface assembly 10 may be joined to an injection gun I (see, e.g., FIG. 58). As will be described in the following disclosure at FIGS. 59A-59G, the second portion 10b of the hypodermic interface assembly 10 is configured to predictably and controllably separate from the first portion 10a of the hypodermic interface assembly 10 (see, e.g., FIGS. 18A-18B, 19A-19B, and 20A-20B).

With reference to FIG. 21A, the third inner surface portion 42c of the inner surface 42 of the substantially tube-shaped body 34 may define a curved or frustoconical surface that extends into the first passage portion 44a of the passage 44 of the hub 14. A peak of the curved or frustoconical surface defined by the third inner surface portion 42c of the inner surface 42 of the substantially tube-shaped body 34 is defined generally by a dashed line P that is orthogonal to the central axis A₁₀-A₁₀ of the hypodermic interface assembly 10. Furthermore, as also seen at FIG. 21A, another dashed line B that is orthogonal to the central axis A₁₀-A₁₀ of the hypodermic interface assembly 10 extends across the substantially flat outer shoulder surface portion 64b of the outer shoulder surface portion 64 of the outer surface 40 of the substantially tube-shaped body 34. The dashed line B generally demarcates the first portion 34a of the substantially tube-shaped body 34 of the hub 14 from the second portion 34b of the substantially tube-shaped body 34 of the hub 14. Accordingly the dashed line B may be generally referred to as a “break line” at or substantially at, about, along, or on the location where the second portion 34b of the substantially tube-shaped body 34 of the hub 14 generally separates from the first portion 34a of the substantially tube-shaped body 34 of the hub 14 for forming, respectively, the second portion 10b of the hypodermic interface assembly 10 and the first portion 10a of the hypodermic interface assembly 10. Although the break line B is represented in the Figures as described above, the location of the break line B is exemplary. In other words, the predicable failure (break) of the neck 65 of the hub 14 may occur (a) at or proximate the location where the neck 65 of the hub 14 meets the head 67 of the hub 14, (b) at or proximate the location of the break line B as shown in FIG. 21A, or (c) anywhere between these locations. The location of the cannula 12 relative to the hub 14 also may impact the location of the predicable failure (break) of the neck 65 of the hub 14.

As seen at FIG. 21A, and as described above, the proximal end 16_{P} of the tube-shaped body 16 of the cannula 12 is arranged beyond the third inner surface portion 42c of the inner surface 42 of the substantially tube-shaped body 34 and is disposed within the first passage portion 44a of the passage 44 of the hub 14. Accordingly, the proximal end 16_{P} of the tube-shaped body 16 of the cannula 12 may also be said to be arranged beyond, proximal of, or upstream of both of the peak line P (that defines a proximal-most portion of the third inner surface portion 42c of the inner surface 42 of the substantially tube-shaped body 34) and the break line B. Although the cannula 12 may be arranged relative to the hub 14 as seen at FIG. 21A, the cannula 12 may be arranged relative the hub 14 in other configurations as seen at, for example, FIGS. 21B-21G. It should be noted that although the arrangement of the cannula 12 relative the peak line P and break line B of the hub 14 are only illustrated at FIGS. 21A-21G with respect to the hypodermic interface assembly 10, similar arrangements represented at FIGS. 21A-21G may also be applied for arrangements of the cannulas 112, 212, 312, 412 relative the hubs 114, 214, 314, 414 for the corresponding hypodermic interface assemblies 110, 210, 310, 410.

With reference to FIG. 21B, in some configurations, the proximal end 16_{P} of the tube-shaped body 16 of the cannula 12 may be arranged in alignment with the peak line P (that defines a proximal-most portion of the third inner surface portion 42c of the inner surface 42 of the substantially tube-shaped body 34) while being arranged beyond, proximal of, or upstream of the break line B. Referring to FIG. 21C, in other configurations, the proximal end 16_{P} of the tube-shaped body 16 of the cannula 12 may be arranged distal of or downstream of the peak line P (that defines a proximal-most portion of the third inner surface portion 42c of the inner surface 42 of the substantially tube-shaped body 34) while being arranged beyond, proximal of, or upstream of the break line B. As seen at FIG. 21D, in yet other configurations, the proximal end 16_{P} of the tube-shaped body 16 of the cannula 12 may be arranged distal of or downstream of the peak line P (that defines a proximal-most portion of the third inner surface portion 42c of the inner surface 42 of the substantially tube-shaped body 34) while being arranged in alignment with the break line B.

With reference to FIG. 21E, in some configurations, the proximal end 16_{P} of the tube-shaped body 16 of the cannula 12 may be arranged distal of or downstream of both of the peak line P and the break line B while being arranged within a sub-length of the hub 14 defined by the length L₁₄ₐ₂ of the outer neck surface portion 66 of the outer surface 40 of the substantially tube-shaped body 34 of the hub 14. Referring to FIG. 21F, in some configurations, the proximal end 16_{P} of the tube-shaped body 16 of the cannula 12 may be arranged distal of or downstream of both of the peak line P and the break line B while being arranged in alignment with the intermediate surface portion 70 of the outer surface 40 of the substantially tube-shaped body 34. As seen at FIG. 21G, in yet other configurations, the proximal end 16_{P} of the tube-shaped body 16 of the cannula 12 may be arranged distal of or downstream of both of the peak line P and the break line B while being arranged within a sub-length of the hub 14 defined by the length L₁₄ₐ₁ of the outer head surface portion 68 and/or crimping pockets 68′ that infer material deformation of the outer head surface portion 68 of the outer surface 40 of the substantially tube-shaped body 34 of the hub 14.

By arranging the proximal end 16_{P} of the tube-shaped body 16 of the cannula 12 closer to the distal end surface 38 of the hub 14 (as seen, comparatively at FIGS. 21B-21G with respect to, for example, FIG. 21A), a location of stress concentration, which may be alternatively referred to as a stress riser that influences a stress pattern of the hub 14, applied to the cannula 12 arising from the radial force X_{R} (see, e.g., FIG. 59D) imparted to the cannula 12 is also axially moved away from the first portion 10a of the hypodermic interface assembly 10 and closer to the subject S, which may be defined for example, at a location near or at the distal end surface 38 of the hub 14. Accordingly, depending on the desired rigidly of the hypodermic interface assembly 10 (and, correspondingly, the location of stress concentration applied to the cannula 12), a portion of the tube-shaped body 16 of the cannula 12 may be arranged: (1) upstream of the break line B, thereby traversing the break line B (as seen at, e.g., FIGS. 21A-21C); (2) at the break line B (as seen at, e.g., FIG. 21D); or downstream of the break line B (as seen at, e.g., FIGS. 21E-21G).

Irrespective of the location of the cannula 12 relative to the hub 14, in some configurations, the cannula 12 is secured (e.g., by punching, crimping, swaging, materially deforming 68′, or adhering with adhesive (not shown)) to the hub 14 at a region of the second portion 34b of the substantially tube-shaped body 34 of the hub 14 and not at any of the first portion 34a of the substantially tube-shaped body 34 of the hub 14. That is, the cannula 12 may not: (1) be considered to be a component of the first portion 10a of the hypodermic interface assembly 10; or (2) mechanically joined or connected to the first portion 10a of the hypodermic interface assembly 10. Accordingly, the cannula 12 may: (1) be considered to be a component of the second portion 10b of the hypodermic interface assembly 10; or (2) mechanically joined or connected to the second portion 10b (that also includes the second portion 34b of the substantially tube-shaped body 34 of the hub 14 of the hypodermic interface assembly 10). Therefore, when the hypodermic interface assembly 10 predictably separates at or substantially at, about, along, or on the break line B, the arrangement of the cannula 12 relative the hub 14 results in the proximal end 16_{P} of the tube-shaped body 16 of the cannula 12 being “ripped” out of, or, alternatively, being “migrated away from” the first portion 10a of the hypodermic interface assembly 10 while the tube-shaped body 16 of the cannula 12 remains joined to the second portion 10b of the hypodermic interface assembly 10.

With reference to FIGS. 22-30A, 31-39A, 40-48A, and 49-57A, exemplary hypodermic interface assemblies are shown generally at, respectively reference numerals 110 (see, e.g., FIGS. 22-30A), 210 (see, e.g., FIGS. 31-39A), 310 (see, e.g., FIGS. 40-48A), and 410 (see, e.g., FIGS. 49-57A). Each of these hypodermic interface assemblies 110, 210, 310, 410 include, respectively, a cannula 112, 212, 312, 412 and a hub 114, 214, 314, 414. In view of the substantial similarity in structure and function of the components associated with the hypodermic interface assembly 10 with respect to the hypodermic interface assemblies 110, 210, 310, 410, like reference numerals are used hereinafter and in the drawings to identify like components while like reference numerals containing an ‘incremented century’ prefix are used to identify those components (e.g., for the cannula 12, an ‘incremented century’ prefix including the numbers “1”, “2”, “3”, and “4” has been added to the reference numeral of a corresponding cannula (i.e., see comparatively, e.g., the cannulas 112 (including reference numeral “1” arranged directly in front of the reference numeral “12”), 212 (including reference numeral “2” arranged directly in front of the reference numeral “12”), 312 (including reference numeral “3” arranged directly in front of the reference numeral “12”), and 412 (including reference numeral “4” arranged directly in front of the reference numeral “12”)) associated with the hypodermic interface assemblies 110, 210, 310, 410.

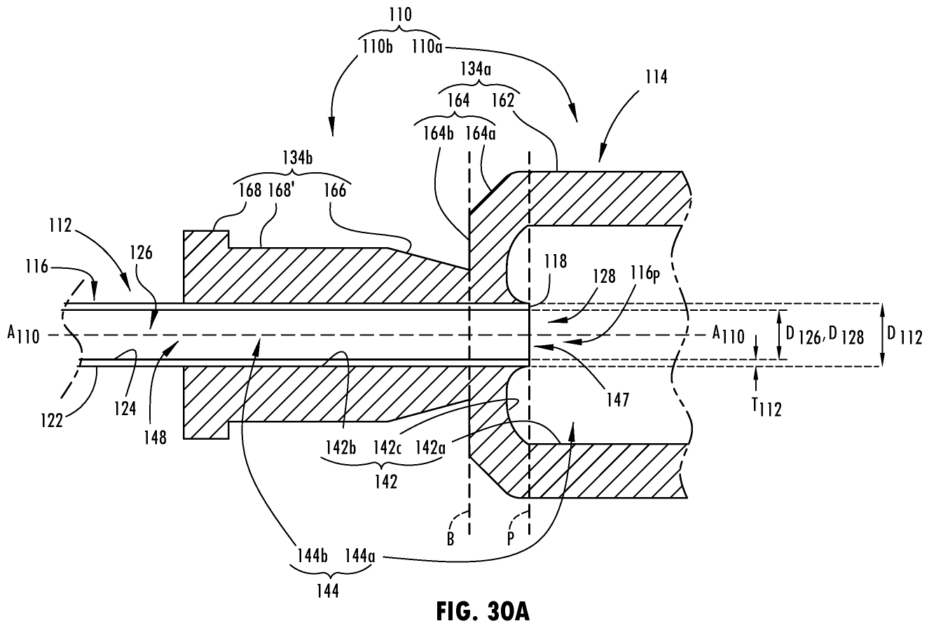

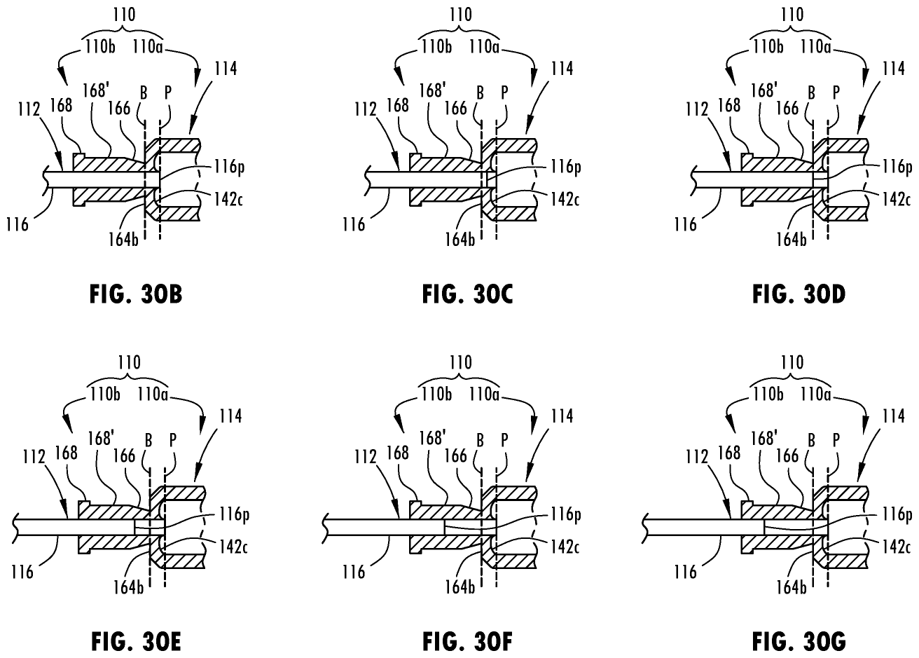

As described above with respect to the exemplary structure of the hub 14 of the hypodermic interface assembly 10, the outer neck surface portion 66 of the outer surface 40 of the substantially tube-shaped body 34 of the hub 14 is substantially perpendicular or orthogonal with respect to both of and connects: (1) the substantially flat outer shoulder surface portion 64b of the outer shoulder surface portion 64 of the outer surface 40 of the substantially tube-shaped body 34; and (2) the intermediate surface portion 70 of the outer surface 40 of the substantially tube-shaped body 34. However, referring to FIGS. 22-30A, although the hypodermic interface assembly 110 is substantially similar to the hypodermic interface assembly 10, the outer neck surface portion 166 of the outer surface 140 of the substantially tube-shaped body 134 of the hub 114 is not substantially perpendicular or orthogonal with respect to the substantially flat outer shoulder surface portion 164b of the outer shoulder surface portion 164 of the outer surface 140 of the substantially tube-shaped body 134. In some configurations, the outer neck surface portion 166 of the outer surface 140 of the substantially tube-shaped body 134 of the hub 114 extends from the substantially flat outer shoulder surface portion 164b of the outer shoulder surface portion 164 of the outer surface 140 of the substantially tube-shaped body 134 at an angle θ₁₆₅. Referring to FIG. 26, the angle θ₁₆₅ may be approximately equal to, for example, 70°; in other configurations, the angle θ₁₆₅ may be any desirable angle ranging between approximately 45° and 85°. Referring also to FIG. 30A, the break line B that defines predictable separation of the second portion 110b of the hypodermic interface assembly 110 from the first portion 110a of the hypodermic interface assembly 110 is seen aligned with and extending across the substantially flat outer shoulder surface portion 164b of the outer shoulder surface portion 164 of the outer surface 140 of the substantially tube-shaped body 134 (as similarly seen above at FIG. 21A). The thickness T₁₆₅ of the neck 165 may be any desirable dimension or in a ratio. In some instances, the thickness T₁₆₅ of the neck 165 may be in a range between approximately 0.10 and 0.95 times a thickness T₁₆₉ of the head 167. In other configurations, the thickness T₁₆₅ of the neck 165 may be in a range between approximately 0.20 and 0.80 times the thickness T₁₆₉ of the head 167. In yet other configurations, the thickness T₁₆₅ of the neck 165 may be in a range between approximately 0.33 and 0.66 times the thickness T₁₆₉ of the head 167.

As seen at FIG. 30A, and as described above, the proximal end 116_{P} of the tube-shaped body 116 of the cannula 112 may arranged beyond the third inner surface portion 142c of the inner surface 142 of the substantially tube-shaped body 134 and is disposed within the first passage portion 144a of the passage 144 of the hub 114. Accordingly, the proximal end 116_{P} of the tube-shaped body 116 of the cannula 112 may also be said to be arranged beyond, proximal of, or upstream of both of the peak line P (that defines a proximal-most portion of the third inner surface portion 142c of the inner surface 142 of the substantially tube-shaped body 134) and the break line B. Although the cannula 112 may be arranged relative to the hub 114 as seen at FIG. 30A, the cannula 112 may be arranged relative the hub 114 in other configurations as seen at, for example, FIGS. 30B-30G.

With reference to FIG. 30B, in some configurations, the proximal end 116_{P} of the tube-shaped body 116 of the cannula 112 may be arranged in alignment with the peak line P (that defines a proximal-most portion of the third inner surface portion 142c of the inner surface 142 of the substantially tube-shaped body 134) while being arranged beyond, proximal of, or upstream of the break line B. Referring to FIG. 30C, in other configurations, the proximal end 116_{P} of the tube-shaped body 116 of the cannula 112 may be arranged distal of or downstream of the peak line P (that defines a proximal-most portion of the third inner surface portion 142c of the inner surface 142 of the substantially tube-shaped body 134) while being arranged beyond, proximal of, or upstream of the break line B. As seen at FIG. 30D, in yet other configurations, the proximal end 116_{P} of the tube-shaped body 116 of the cannula 112 may be arranged distal of or downstream of the peak line P (that defines a proximal-most portion of the third inner surface portion 142c of the inner surface 142 of the substantially tube-shaped body 134) while being arranged in alignment with the break line B.

With reference to FIG. 30E, in some configurations, the proximal end 116_{P} of the tube-shaped body 116 of the cannula 112 may be arranged distal of or downstream of both of the peak line P and the break line B while being arranged within a sub-length of the hub 114 defined by the length L₁₁₄ₐ₂ of the outer neck surface portion 166 of the outer surface 140 of the substantially tube-shaped body 134 of the hub 114. Referring to FIG. 30F, in some configurations, the proximal end 116_{P} of the tube-shaped body 116 of the cannula 112 may be arranged distal of or downstream of both of the peak line P and the break line B while being arranged in alignment with the intermediate surface portion 170 of the outer surface 140 of the substantially tube-shaped body 134. As seen at FIG. 30G, in yet other configurations, the proximal end 116_{P} of the tube-shaped body 116 of the cannula 112 may be arranged distal of or downstream of both of the peak line P and the break line B while being arranged within a sub-length of the hub 114 defined by the length L₁₁₄ₐ₁ of the outer head surface portion 168 and/or crimping pockets 168′ that infer material deformation of the outer head surface portion 168 of the outer surface 140 of the substantially tube-shaped body 134 of the hub 114.

By arranging the proximal end 116_{P} of the tube-shaped body 116 of the cannula 112 closer to the distal end surface 138 of the hub 114 (as seen, comparatively at FIGS. 30B-30G with respect to, for example, FIG. 30A), a location of stress concentration, which may be alternatively referred to as a stress riser that influences a stress pattern of the hub 114, applied to the cannula 112 arising from the radial force X_{R} (see, e.g., FIG. 59D) imparted to the cannula 112 is also axially moved away from the first portion 110a of the hypodermic interface assembly 110 and closer to the subject S, which may be defined for example, at a location near or at the distal end surface 138 of the hub 114. Accordingly, depending on the desired rigidly of the hypodermic interface assembly 110 (and, correspondingly, the location of stress concentration applied to the cannula 112), a portion of the tube-shaped body 116 of the cannula 112 may be arranged: (1) upstream of the break line B, thereby traversing the break line B (as seen at, e.g., FIGS. 30A-30C); (2) at the break line B (as seen at, e.g., FIG. 30D); or downstream of the break line B (as seen at, e.g., FIGS. 30E-30G).

Irrespective of the location of the cannula 112 relative to the hub 114, in some configurations, the cannula 112 is secured (e.g., by punching, crimping, swaging, materially deforming 168′, or adhering with adhesive (not shown)) to the hub 114 at a region of the second portion 134b of the substantially tube-shaped body 134 of the hub 114 and not at any of the first portion 134a of the substantially tube-shaped body 134 of the hub 114. That is, the cannula 112 may not: (1) be considered to be a component of the first portion 110a of the hypodermic interface assembly 110; or (2) mechanically joined or connected to the first portion 110a of the hypodermic interface assembly 110. Accordingly, the cannula 112 may: (1) be considered to be a component of the second portion 110b of the hypodermic interface assembly 110; or (2) mechanically joined or connected to the second portion 110b (that also includes the second portion 134b of the substantially tube-shaped body 134 of the hub 114 of the hypodermic interface assembly 110). Therefore, when second portion 110b of the hypodermic interface assembly 110 predictably separates from first portion 110a of the hypodermic interface assembly 110, the arrangement of the cannula 112 relative the hub 114 results in the proximal end 116_{P} of the tube-shaped body 116 of the cannula 112 being “ripped” out of, or, alternatively, being “migrated away from” the first portion 110a of the hypodermic interface assembly 110 while the tube-shaped body 116 of the cannula 112 remains joined to the second portion 110b of the hypodermic interface assembly 110.

Furthermore, a distal-most end of the outer neck surface portion 166 of the outer surface 140 of the substantially tube-shaped body 134 of the hub 114 does not terminate at an intermediate surface portion of the outer surface 140 of the substantially tube-shaped body 134. That is, the second portion 134b of the substantially tube-shaped body 134 of the hub 114 does not include an intermediate surface portion of the outer surface 140 of the substantially tube-shaped body 134 (see, comparatively, e.g., the intermediate surface portion 70 of the outer surface 40 of the substantially tube-shaped body 34 of the hypodermic interface assembly 10). Instead, the outer neck surface portion 166 of the outer surface 140 of the substantially tube-shaped body 134 of the hub 114 terminates at a proximal-most end of the outer head surface portion 168 of the outer surface 140 of the substantially tube-shaped body 134 of the hub 114.

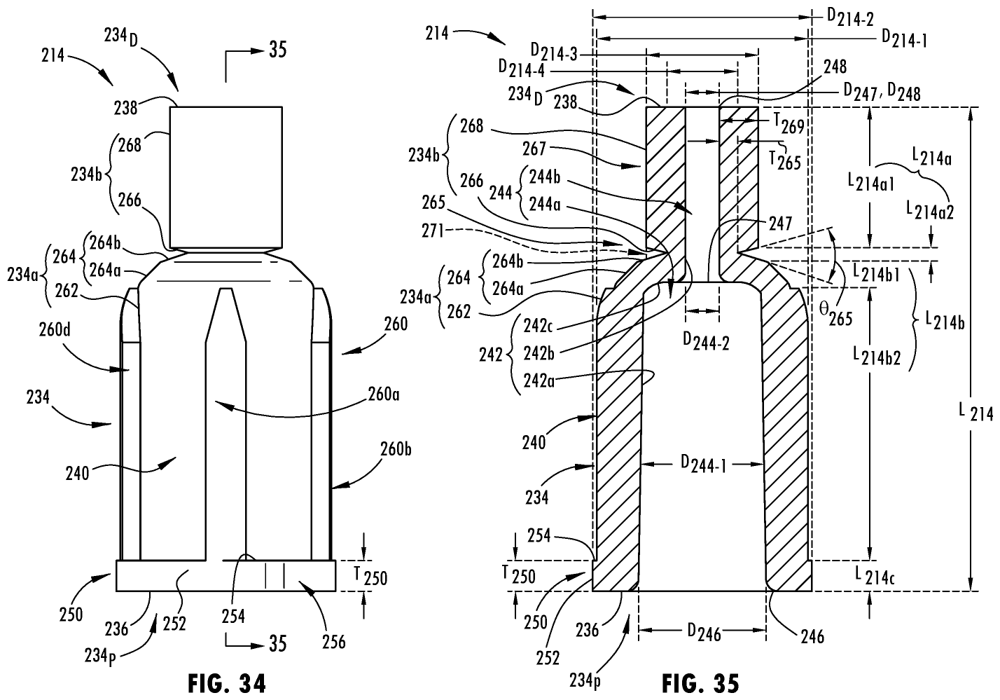

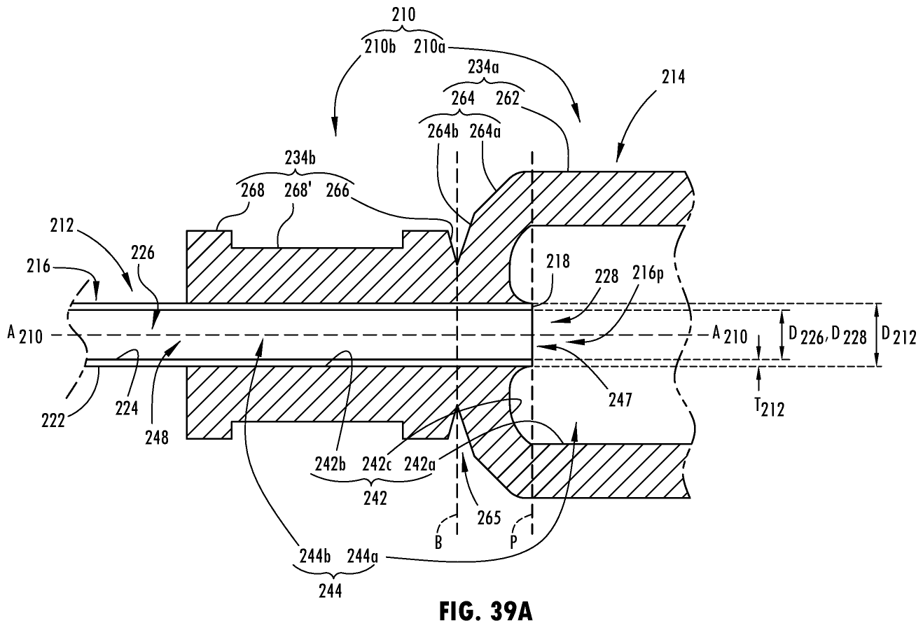

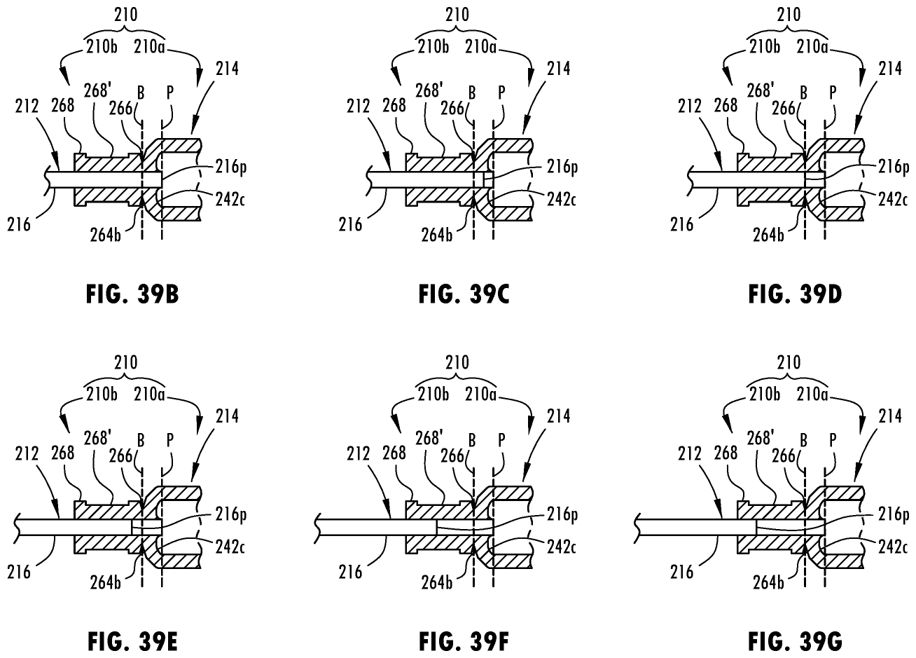

Referring to FIGS. 31-39A, the hypodermic interface assembly 210 is substantially similar to the hypodermic interface assembly 110 with the exception that the substantially flat outer shoulder surface portion 264b of the outer shoulder surface portion 264 of the outer surface 240 of the substantially tube-shaped body 234 is not substantially perpendicular or orthogonal to the central axis A₂₁₀-A₂₁₀ of the hypodermic interface assembly 210. With reference to FIGS. 34-35, in some configurations, the substantially flat outer shoulder surface portion 264b of the outer shoulder surface portion 264 of the outer surface 240 of the substantially tube-shaped body 234 may be symmetrically arranged with respect to an angle of the outer neck surface portion 266 of the outer surface 240 of the substantially tube-shaped body 234 of the hub 214. Accordingly, the substantially flat outer shoulder surface portion 264b and the outer neck surface portion 266 cooperate to form a circumferential V-shaped notch or groove 265 that defines an angle θ₂₆₅ (see, e.g., FIG. 35). The angle θ₂₆₅ may be approximately equal to, for example, 45°. Referring also to FIG. 39A, the break line B that defines predictable separation of the second portion 210b of the hypodermic interface assembly 210 from the first portion 210a of the hypodermic interface assembly 210 is seen aligned with and extends across a valley of the V-shaped notch or groove 265. The thickness T₂₆₅ of the neck 265 may be any desirable dimension or in a ratio. In some instances, the thickness T₂₆₅ of the neck 265 may be in a range between approximately 0.10 and 0.95 times a thickness T₂₆₉ of the head 267. In other configurations, the thickness T₂₆₅ of the neck 265 may be in a range between approximately 0.20 and 0.80 times the thickness T₂₆₉ of the head 267. In yet other configurations, the thickness T₂₆₉ of the neck 265 may be in a range between approximately 0.33 and 0.66 times the thickness T₂₆₉ of the head 267.

As seen at FIG. 39A, and as described above, the proximal end 216_{P} of the tube-shaped body 216 of the cannula 212 is arranged beyond the third inner surface portion 242c of the inner surface 242 of the substantially tube-shaped body 234 and is disposed within the first passage portion 244a of the passage 244 of the hub 214. Accordingly, the proximal end 216_{P} of the tube-shaped body 216 of the cannula 212 may also be said to be arranged beyond, proximal of, or upstream of both of the peak line P (that defines a proximal-most portion of the third inner surface portion 242c of the inner surface 242 of the substantially tube-shaped body 234) and the break line B. Although the cannula 212 may be arranged relative to the hub 214 as seen at FIG. 39A, the cannula 212 may be arranged relative the hub 214 in other configurations as seen at, for example, FIGS. 39B-39G.

With reference to FIG. 39B, in some configurations, the proximal end 216_{P} of the tube-shaped body 216 of the cannula 212 may be arranged in alignment with the peak line P (that defines a proximal-most portion of the third inner surface portion 242c of the inner surface 242 of the substantially tube-shaped body 234) while being arranged beyond, proximal of, or upstream of the break line B. Referring to FIG. 39C, in other configurations, the proximal end 216_{P} of the tube-shaped body 216 of the cannula 212 may be arranged distal of or downstream of the peak line P (that defines a proximal-most portion of the third inner surface portion 242c of the inner surface 242 of the substantially tube-shaped body 234) while being arranged beyond, proximal of, or upstream of the break line B. As seen at FIG. 39D, in yet other configurations, the proximal end 216_{P} of the tube-shaped body 216 of the cannula 212 may be arranged distal of or downstream of the peak line P (that defines a proximal-most portion of the third inner surface portion 242c of the inner surface 242 of the substantially tube-shaped body 234) while being arranged in alignment with the break line B.

With reference to FIG. 39E, in some configurations, the proximal end 216_{P} of the tube-shaped body 216 of the cannula 212 may be arranged distal of or downstream of both of the peak line P and the break line B while being arranged within a sub-length of the hub 214 defined by the length L₂₁₄ₐ₂ of the outer neck surface portion 266 of the outer surface 240 of the substantially tube-shaped body 234 of the hub 214. Referring to FIG. 39F, in some configurations, the proximal end 216_{P} of the tube-shaped body 216 of the cannula 212 may be arranged distal of or downstream of both of the peak line P and the break line B while being arranged in alignment with the intermediate surface portion 270 of the outer surface 240 of the substantially tube-shaped body 234. As seen at FIG. 39G, in yet other configurations, the proximal end 216_{P} of the tube-shaped body 216 of the cannula 212 may be arranged distal of or downstream of both of the peak line P and the break line B while being arranged within a sub-length of the hub 214 defined by the length L₂₁₄ₐ₁ of the outer head surface portion 268 and/or crimping pockets 268′ that infer material deformation of the outer head surface portion 268 of the outer surface 240 of the substantially tube-shaped body 234 of the hub 214.

By arranging the proximal end 216_{P} of the tube-shaped body 216 of the cannula 212 closer to the distal end surface 238 of the hub 214 (as seen, comparatively at FIGS. 39B-39G with respect to, for example, FIG. 39A), a location of stress concentration, which may be alternatively referred to as a stress riser that influences a stress pattern of the hub 214, applied to the cannula 212 arising from the radial force X_{R} (see, e.g., FIG. 59D) imparted to the cannula 212 is also axially moved away from the first portion 210a of the hypodermic interface assembly 210 and closer to the subject S, which may be defined for example, at a location near or at the distal end surface 238 of the hub 214. Accordingly, depending on the desired rigidly of the hypodermic interface assembly 210 (and, correspondingly, the location of stress concentration applied to the cannula 212), a portion of the tube-shaped body 216 of the cannula 212 may be arranged: (1) upstream of the break line B, thereby traversing the break line B (as seen at, e.g., FIGS. 39A-39C); (2) at the break line B (as seen at, e.g., FIG. 39D); or downstream of the break line B (as seen at, e.g., FIGS. 39E-39G).

Irrespective of the location of the cannula 212 relative to the hub 214, in some configurations, the cannula 212 is secured (e.g., by punching, crimping, swaging, materially deforming 268′, or adhering with adhesive (not shown)) to the hub 214 at a region of the second portion 234b of the substantially tube-shaped body 234 of the hub 214 and not at any of the first portion 234a of the substantially tube-shaped body 234 of the hub 214. That is, the cannula 212 may not: (1) be considered to be a component of the first portion 210a of the hypodermic interface assembly 210; or (2) mechanically joined or connected to the first portion 210a of the hypodermic interface assembly 210. Accordingly, the cannula 212 may: (1) be considered to be a component of the second portion 210b of the hypodermic interface assembly 210; or (2) mechanically joined or connected to the second portion 210b (that also includes the second portion 234b of the substantially tube-shaped body 234 of the hub 214 of the hypodermic interface assembly 210). Therefore, when the second portion 210b of the hypodermic interface assembly 210 predictably separates from the first portion 210a of the hypodermic interface assembly 210, the arrangement of the cannula 212 relative the hub 214 results in the proximal end 216_{P} of the tube-shaped body 216 of the cannula 212 being “ripped” out of, or, alternatively, being “migrated away from” the first portion 210a of the hypodermic interface assembly 210 while the tube-shaped body 216 of the cannula 212 remains joined to the second portion 210b of the hypodermic interface assembly 210.

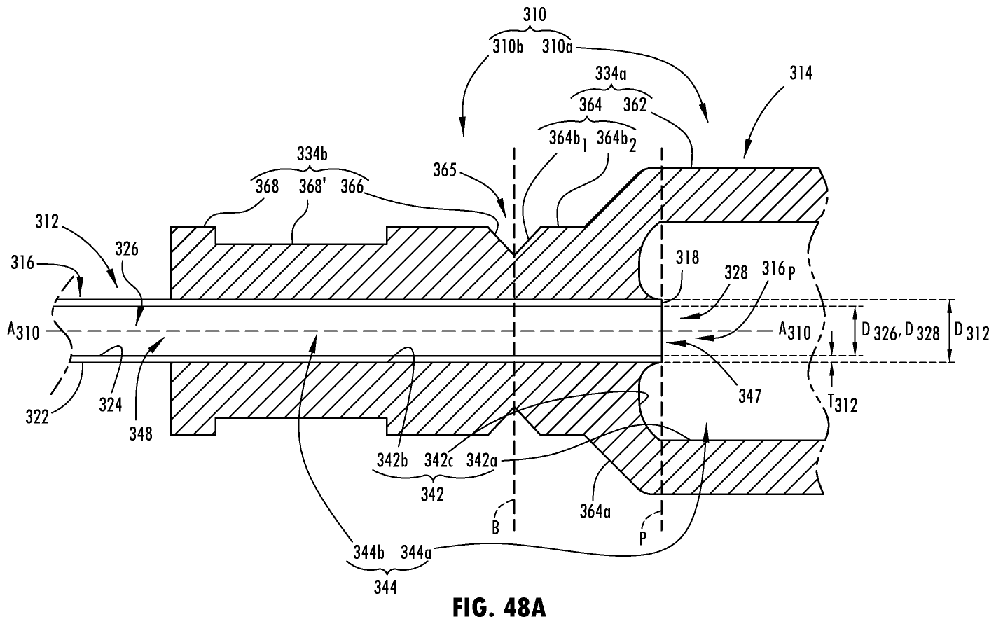

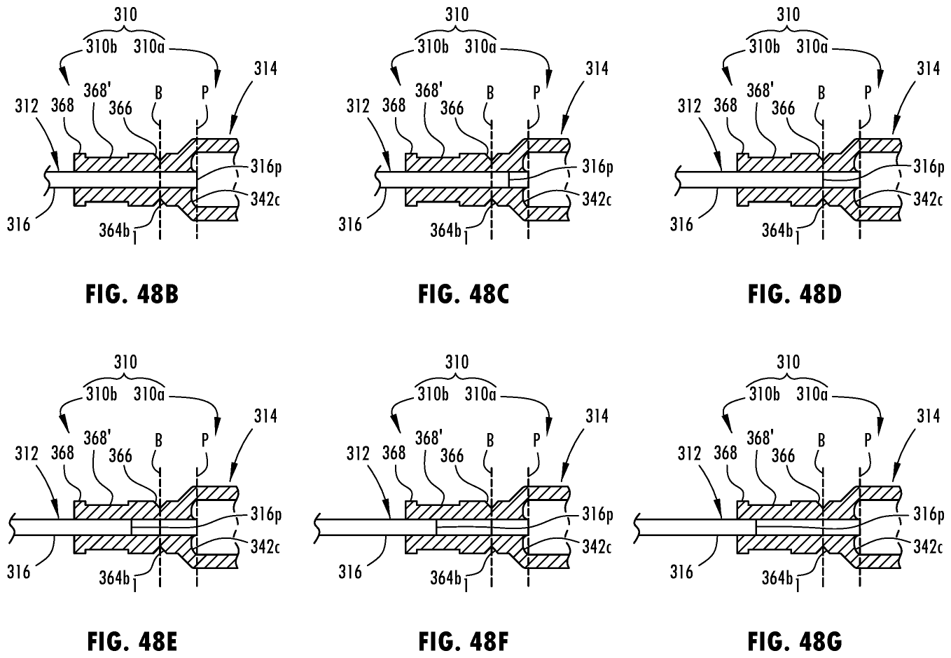

Referring to FIGS. 40-48A, the hypodermic interface assembly 310 is substantially similar to the hypodermic interface assembly 210 such that the hub 314 also includes V-shaped notch or groove 365 that defines an angle θ₃₆₅ (see, e.g., FIG. 44). The angle θ₃₆₅ may, however, be not be as shallow as the V-shaped notch or groove 265, and, as such, may be defined by a larger angle that may be approximately equal to, for example, 90°. Referring also to FIG. 48A, the break line B that defines predictable separation of the second portion 310b of the hypodermic interface assembly 310 from the first portion 310a of the hypodermic interface assembly 310 is seen aligned with and extending across a valley of the V-shaped notch or groove 365.

As seen at FIG. 48A, and as described above, the proximal end 316_{P} of the tube-shaped body 316 of the cannula 312 is arranged beyond the third inner surface portion 342c of the inner surface 342 of the substantially tube-shaped body 334 and is disposed within the first passage portion 344a of the passage 344 of the hub 314. Accordingly, the proximal end 316_{P} of the tube-shaped body 316 of the cannula 312 may also be said to be arranged beyond, proximal of, or upstream of both of the peak line P (that defines a proximal-most portion of the third inner surface portion 342c of the inner surface 342 of the substantially tube-shaped body 334) and the break line B. Although the cannula 312 may be arranged relative to the hub 314 as seen at FIG. 48A, the cannula 312 may be arranged relative the hub 314 in other configurations as seen at, for example, FIGS. 48B-48G.

With reference to FIG. 48B, in some configurations, the proximal end 316_{P} of the tube-shaped body 316 of the cannula 312 may be arranged in alignment with the peak line P (that defines a proximal-most portion of the third inner surface portion 342c of the inner surface 342 of the substantially tube-shaped body 334) while being arranged beyond, proximal of, or upstream of the break line B. Referring to FIG. 48C, in other configurations, the proximal end 316_{P} of the tube-shaped body 316 of the cannula 312 may be arranged distal of or downstream of the peak line P (that defines a proximal-most portion of the third inner surface portion 342c of the inner surface 342 of the substantially tube-shaped body 334) while being arranged beyond, proximal of, or upstream of the break line B. As seen at FIG. 48D, in yet other configurations, the proximal end 316_{P} of the tube-shaped body 316 of the cannula 312 may be arranged distal of or downstream of the peak line P (that defines a proximal-most portion of the third inner surface portion 342c of the inner surface 342 of the substantially tube-shaped body 334) while being arranged in alignment with the break line B.

With reference to FIG. 48E, in some configurations, the proximal end 316_{P} of the tube-shaped body 316 of the cannula 312 may be arranged distal of or downstream of both of the peak line P and the break line B while being arranged within a sub-length of the hub 314 defined by the length L₃₁₄ₐ₂ of the outer neck surface portion 366 of the outer surface 340 of the substantially tube-shaped body 334 of the hub 314. Referring to FIG. 48F, in some configurations, the proximal end 316_{P} of the tube-shaped body 316 of the cannula 312 may be arranged distal of or downstream of both of the peak line P and the break line B while being arranged in alignment with the intermediate surface portion 370 of the outer surface 340 of the substantially tube-shaped body 334. As seen at FIG. 48G, in yet other configurations, the proximal end 316_{P} of the tube-shaped body 316 of the cannula 312 may be arranged distal of or downstream of both of the peak line P and the break line B while being arranged within a sub-length of the hub 314 defined by the length L₃₁₄ₐ₁ of the outer head surface portion 368 and/or crimping pockets 368′ that infer material deformation of the outer head surface portion 368 of the outer surface 340 of the substantially tube-shaped body 334 of the hub 314. The thickness T₃₆₅ of the neck 365 may be any desirable dimension or in a ratio. In some instances, the thickness T₃₆₅ of the neck 365 may be in a range between approximately 0.15 and 0.85 times a thickness T₃₆₉ of the head 367. In other configurations, the thickness T₃₆₅ of the neck 365 may be in a range between approximately 0.25 and 0.75 times the thickness T₃₆₉ of the head 367. In yet other configurations, the thickness T₃₆₅ of the neck 367 may be in a range between approximately 0.33 and 0.66 times the thickness T₃₆₉ of the head 367.

By arranging the proximal end 316_{P} of the tube-shaped body 316 of the cannula 312 closer to the distal end surface 338 of the hub 314 (as seen, comparatively at FIGS. 48B-48G with respect to, for example, FIG. 48A), a location of stress concentration, which may be alternatively referred to as a stress riser that influences a stress pattern of the hub 314, applied to the cannula 312 arising from the radial force X_{R} (see, e.g., FIG. 59D) imparted to the cannula 312 is also axially moved away from the first portion 310a of the hypodermic interface assembly 310 and closer to the subject S, which may be defined for example, at a location near or at the distal end surface 338 of the hub 314. Accordingly, depending on the desired rigidly of the hypodermic interface assembly 310 (and, correspondingly, the location of stress concentration applied to the cannula 312), a portion of the tube-shaped body 316 of the cannula 312 may be arranged: (1) upstream of the break line B, thereby traversing the break line B (as seen at, e.g., FIGS. 48A-48C); (2) at the break line B (as seen at, e.g., FIG. 48D); or downstream of the break line B (as seen at, e.g., FIGS. 48E-48G).

Irrespective of the location of the cannula 312 relative to the hub 314, in some configurations, the cannula 312 is secured (e.g., by punching, crimping, swaging, materially deforming 368′, or adhering with adhesive (not shown)) to the hub 314 at a region of the second portion 334b of the substantially tube-shaped body 334 of the hub 314 and not at any of the first portion 334a of the substantially tube-shaped body 334 of the hub 314. That is, the cannula 312 may not: (1) be considered to be a component of the first portion 310a of the hypodermic interface assembly 310; or (2) mechanically joined or connected to the first portion 310a of the hypodermic interface assembly 310. Accordingly, the cannula 312 may: (1) be considered to be a component of the second portion 310b of the hypodermic interface assembly 310; or (2) mechanically joined or connected to the second portion 310b (that also includes the second portion 334b of the substantially tube-shaped body 334 of the hub 314 of the hypodermic interface assembly 310). Therefore, when the second portion 310b of the hypodermic interface assembly 310 predictably separates from the first portion 310a of the hypodermic interface assembly 310, the arrangement of the cannula 312 relative the hub 314 results in the proximal end 316_{P} of the tube-shaped body 316 of the cannula 312 being “ripped” out of, or, alternatively, being “migrated away from” the first portion 310a of the hypodermic interface assembly 310 while the tube-shaped body 316 of the cannula 312 remains joined to the second portion 310b of the hypodermic interface assembly 310.

Furthermore, the hypodermic interface assembly 310 may be defined by an “extended head” portion. The extended head portion of the hypodermic interface assembly 310 may be defined by a length L₃₁₄ₐ₁ (see FIG. 44) of the outer head surface portion 368 of the outer surface 340 of the substantially tube-shaped body 334 of the hub 314 that may be approximately about, for example, six (6) times longer than a length L₃₁₄ₐ₂ of the outer neck surface portion 366 of the outer surface 340 of the substantially tube-shaped body 334 of the hub 314.

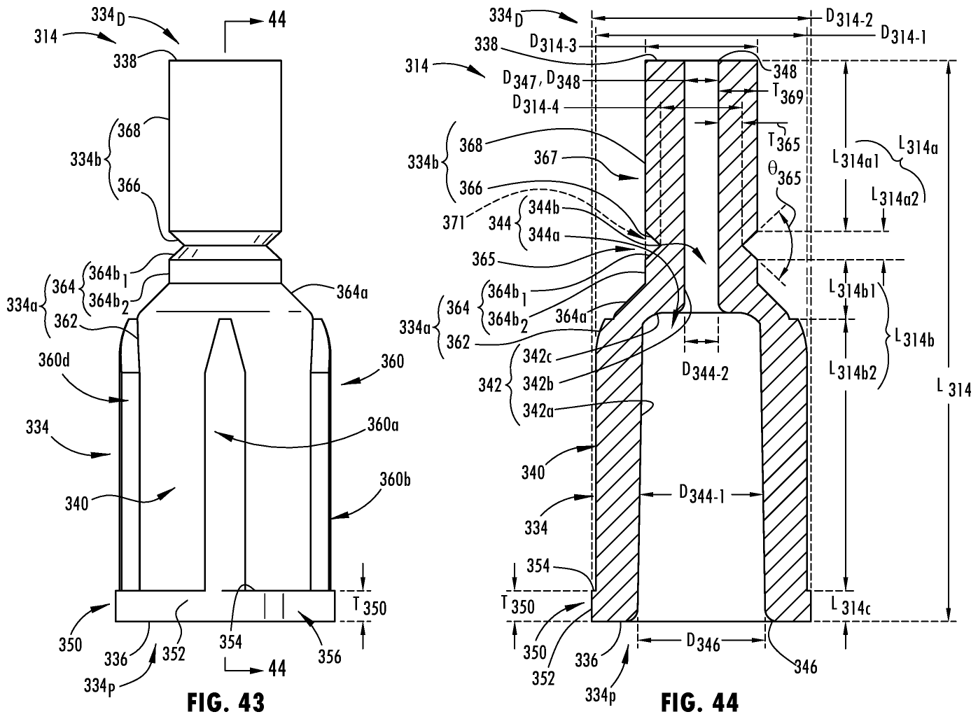

Yet even further, as seen at, for example, FIG. 43, the substantially flat outer shoulder surface portion 364 may be defined by a first substantially flat outer shoulder surface portion 364b₁ and a second substantially flat outer shoulder surface portion 364b₂. The first substantially flat outer shoulder surface portion 364b₁ is arranged substantially similarly with respect to the substantially flat outer shoulder surface portion 264b described above. The second substantially flat outer shoulder surface portion 364b₂, however, circumscribes the central axis A₃₁₀-A₃₁₀ of the hypodermic interface assembly 310 in a substantially concentric fashion and may be axially aligned with the outer head surface portion 368 of the outer surface 340 of the substantially tube-shaped body 334 of the hub 314.

Referring to FIGS. 49-57A, the hypodermic interface assembly 410 is substantially similar to the hypodermic interface assemblies 210, 310 such that the hub 414 also includes V-shaped notch or groove 465 that defines an angle θ₄₆₅ (see, e.g., FIG. 53). The angle θ₄₆₅ may be approximately equal to, for example, 45°; in other configurations, the angle θ₄₆₅ may be any desirable angle ranging between approximately −45° (i.e., an undercut angle) and +120° (i.e., a slope). The thickness T₄₆₅ of the substantially “dome-and-V-shaped-groove” neck portion (defined by a combination of a dome-shaped surface portion 464a and the V-shaped notch or groove 465 extending along the length L₄₁₄ₐ₂) may be any desirable dimension or in a ratio. In some instances, the thickness T₄₆₅ of the “dome-and-V-shaped-groove” neck portion may be in a range between approximately 0.10 and 1.50 times the thickness T₄₆₉ of the head 467. In other configurations, the thickness T₄₆₅ of the “dome-and-V-shaped-groove” neck portion may be in a range between approximately 0.25 and 0.99 times the thickness T₄₆₉ of the head 467. In yet other configurations, the thickness T₄₆₅ of the “dome-and-V-shaped-groove” neck portion may be in a range between approximately 0.33 and 0.66 times the thickness T₄₆₉ of the head 467.

Furthermore, as seen at FIG. 53, the V-shaped notch or groove 465 may define an absence of space or a depth 471 of the hub 414. As also seen in the present disclosure at FIGS. 7, 26, 35, and 44, other exemplary hubs 114, 214, 314, and 414 may also define substantially similar depths at 71, 171, 271, and 371. The depth 471 may be any desirable dimension or in a ratio. In some instances, the depth 471 may be in a range between approximately 0.15 and 0.95 times the outer diameter D₄₁₄₋₃. In other configurations, the depth 471 may be in a range between approximately 0.25 and 0.85 times the outer diameter D₄₁₄₋₃. In yet other configurations, the depth 471 may be in a range between approximately 0.30 and 0.75 times the outer diameter D₄₁₄₋₃. Unlike the hypodermic interface assemblies 210, 310 described above, the V-shaped notch or groove 465 is axially shifted away from the distal end surface 438 of the hub 414 and is arranged axially closer to the proximal end surface 436 of the substantially tube-shaped body 434 of the hub 414. In some configurations, the V-shaped notch or groove 465 extends into the substantially tube-shaped body 434 of the hub 414 between a distal-most end of the outer body surface portion 462 of the outer surface 440 of the substantially tube-shaped body 434 and a proximal-most end of the outer body surface portion 462 of the outer surface 440 of the substantially tube-shaped body 434.

Furthermore, the hypodermic interface assembly 410 does not include: (1) a substantially flat outer shoulder surface portion (see comparatively, e.g., reference numerals 64b, 164b, 264b, 364b); (2) an outer neck surface portion (see comparatively, e.g., reference numerals 66, 166, 266, 366); and (3) an intermediate surface portion (see comparatively, e.g., reference numerals 70). Accordingly, a proximal-most end of the outer head surface portion 468 of the outer surface 440 of the substantially tube-shaped body 434 extends from a distal-most end of the dome-shaped or curved outer shoulder surface portion 464a of the outer surface 440 of the substantially tube-shaped body 434. As such, the first portion 434a of the substantially tube-shaped body 434 of the hub 414 may be defined by the outer body surface portion 462 of the outer surface 440 of the substantially tube-shaped body 434, and the second portion 434b of the substantially tube-shaped body 434 of the hub 414 may be defined by: the dome-shaped or curved outer shoulder surface portion 464a of the outer surface 440 of the substantially tube-shaped body 434 and the outer head surface portion 468 of the outer surface 440 of the substantially tube-shaped body 434.

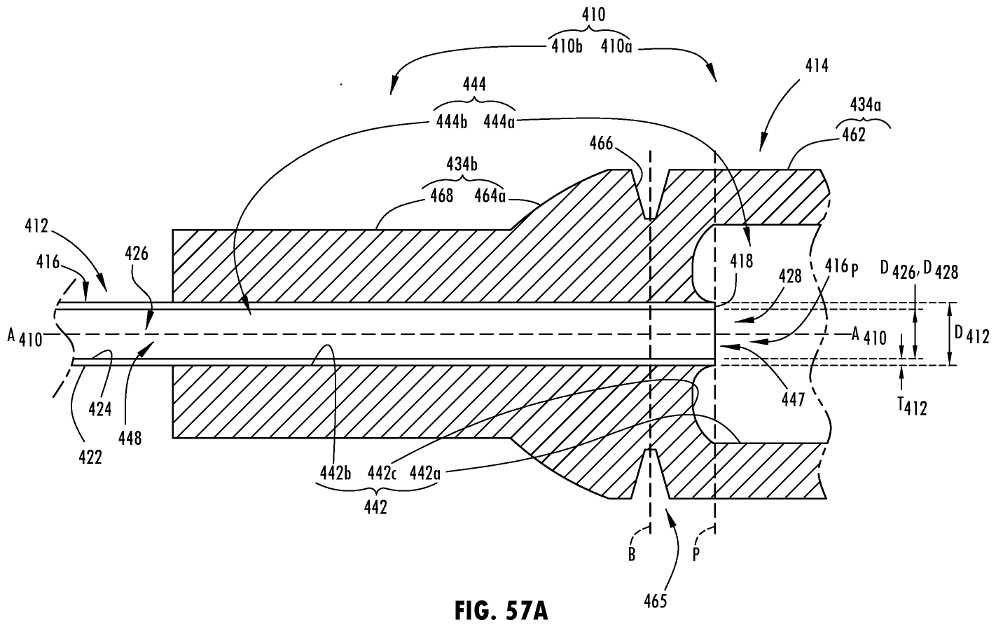

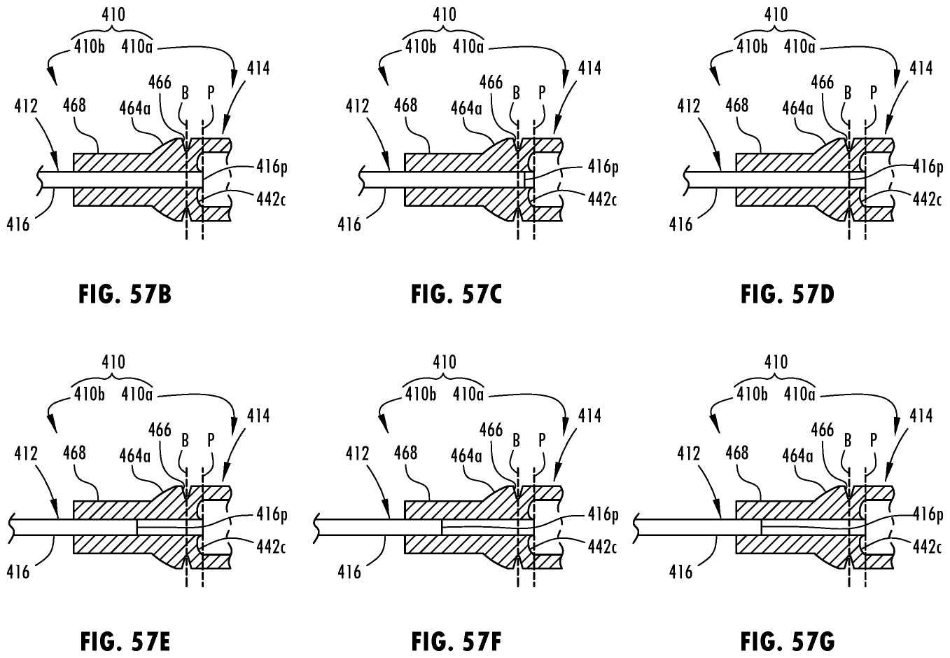

Referring also to FIG. 57A, the break line B that defines predictable separation of the second portion 410b of the hypodermic interface assembly 410 from the first portion 410a of the hypodermic interface assembly 410 is seen aligned with and extending across a valley of the V-shaped notch or groove 465. Because the break line B is arranged proximal of the dome-shaped or curved outer shoulder surface portion 464a of the outer surface 440 of the substantially tube-shaped body 434, and, because the distal-most end of the dome-shaped or curved outer shoulder surface portion 464a of the outer surface 440 of the substantially tube-shaped body 434 extends directly from the proximal-most end of the outer head surface portion 468 of the outer surface 440 of the substantially tube-shaped body 434, unlike the exemplary hypodermic interface assemblies 10, 110, 210, 310 described above, the dome-shaped or curved outer shoulder surface portion 464a of the outer surface 440 of the substantially tube-shaped body 434 may define a portion of the second portion 410b of the hypodermic interface assembly 410.

As seen at FIG. 57A, and as described above, the proximal end 416_{P} of the tube-shaped body 416 of the cannula 412 is arranged beyond the third inner surface portion 442c of the inner surface 442 of the substantially tube-shaped body 434 and is disposed within the first passage portion 444a of the passage 444 of the hub 414. Accordingly, the proximal end 416_{P} of the tube-shaped body 416 of the cannula 412 may also be said to be arranged beyond, proximal of, or upstream of both of the peak line P (that defines a proximal-most portion of the third inner surface portion 442c of the inner surface 442 of the substantially tube-shaped body 434) and the break line B. Although the cannula 412 may be arranged relative to the hub 414 as seen at FIG. 57A, the cannula 412 may be arranged relative the hub 414 in other configurations as seen at, for example, FIGS. 57B-57G.

With reference to FIG. 57B, in some configurations, the proximal end 416_{P} of the tube-shaped body 416 of the cannula 412 may be arranged in alignment with the peak line P (that defines a proximal-most portion of the third inner surface portion 442c of the inner surface 442 of the substantially tube-shaped body 434) while being arranged beyond, proximal of, or upstream of the break line B. Referring to FIG. 57C, in other configurations, the proximal end 416_{P} of the tube-shaped body 416 of the cannula 412 may be arranged distal of or downstream of the peak line P (that defines a proximal-most portion of the third inner surface portion 442c of the inner surface 442 of the substantially tube-shaped body 434) while being arranged beyond, proximal of, or upstream of the break line B. As seen at FIG. 57D, in yet other configurations, the proximal end 416_{P} of the tube-shaped body 416 of the cannula 412 may be arranged distal of or downstream of the peak line P (that defines a proximal-most portion of the third inner surface portion 442c of the inner surface 442 of the substantially tube-shaped body 434) while being arranged in alignment with the break line B.

With reference to FIG. 57E, in some configurations, the proximal end 416_{P} of the tube-shaped body 416 of the cannula 412 may be arranged distal of or downstream of both of the peak line P and the break line B while being arranged within a sub-length of the hub 414 defined by the length L₄₁₄ₐ₁ of the outer head surface portion 468 of the outer surface 440 of the substantially tube-shaped body 434 of the hub 414. Referring to FIG. 57F, in some configurations, the proximal end 416_{P} of the tube-shaped body 416 of the cannula 412 may be arranged distal of or downstream of both of the peak line P and the break line B while also being arranged distal of or downstream of the outer neck surface portion 466 of the outer surface 440 of the substantially tube-shaped body 434. As seen at FIG. 57G, in yet other configurations, the proximal end 416_{P} of the tube-shaped body 416 of the cannula 412 may be arranged distal of or downstream of both of the peak line P and the break line B while being arranged within a sub-length of the hub 414 defined by the length L₄₁₄ₐ₁ of the outer head surface portion 468 and/or crimping pockets 468′ that infer material deformation of the outer head surface portion 468 of the outer surface 440 of the substantially tube-shaped body 434 of the hub 414.

By arranging the proximal end 416_{P} of the tube-shaped body 416 of the cannula 412 closer to the distal end surface 438 of the hub 414 (as seen, comparatively at FIGS. 57B-57G with respect to, for example, FIG. 57A), a location of stress concentration, which may be alternatively referred to as a stress riser that influences a stress pattern of the hub 414, applied to the cannula 412 arising from the radial force X_{R} (see, e.g., FIG. 59D) imparted to the cannula 412 is also axially moved away from the first portion 410a of the hypodermic interface assembly 410 and closer to the subject S, which may be defined for example, at a location near or at the distal end surface 438 of the hub 414. Accordingly, depending on the desired rigidly of the hypodermic interface assembly 410 (and, correspondingly, the location of stress concentration applied to the cannula 412), a portion of the tube-shaped body 416 of the cannula 412 may be arranged: (1) upstream of the break line B, thereby traversing the break line B (as seen at, e.g., FIGS. 57A-57C); (2) at the break line B (as seen at, e.g., FIG. 57D); or downstream of the break line B (as seen at, e.g., FIGS. 57E-57G).

Irrespective of the location of the cannula 412 relative to the hub 414, in some configurations, the cannula 412 is secured (e.g., by punching, crimping, swaging, materially deforming 468′, or adhering with adhesive (not shown)) to the hub 414 at a region of the second portion 434b of the substantially tube-shaped body 434 of the hub 414 and not at any of the first portion 434a of the substantially tube-shaped body 434 of the hub 414. That is, the cannula 412 may not: (1) be considered to be a component of the first portion 410a of the hypodermic interface assembly 410; or (2) mechanically joined or connected to the first portion 410a of the hypodermic interface assembly 410. Accordingly, the cannula 412 may: (1) be considered to be a component of the second portion 410b of the hypodermic interface assembly 410; or (2) mechanically joined or connected to the second portion 410b (that also includes the second portion 434b of the substantially tube-shaped body 434 of the hub 414 of the hypodermic interface assembly 410). Therefore, when the second portion 410b of the hypodermic interface assembly 410 predictably separates from the first portion 410a of the hypodermic interface assembly 410, the arrangement of the cannula 412 relative the hub 414 results in the proximal end 416_{P} of the tube-shaped body 416 of the cannula 412 being “ripped” out of, or, alternatively, being “migrated away from” the first portion 410a of the hypodermic interface assembly 410 while the tube-shaped body 416 of the cannula 412 remains joined to the second portion 410b of the hypodermic interface assembly 410.

Referring now to FIGS. 58 and 59A-59G, a methodology for utilizing any of the hypodermic interface assemblies 10 (see, e.g., FIGS. 11C-18B and 21A-21G), 110 (see, e.g., FIGS. 22-30), 210 (see, e.g., FIGS. 31-39), 310 (see, e.g., FIGS. 40-48), and 410 (see, e.g., FIGS. 49-57) is shown. Although FIGS. 58 and 59A-59G show a methodology for utilizing the hypodermic interface assembly 10, any of the other hypodermic interface assemblies 110, 210, 310, 410 described in the present disclosure may also be utilized in a substantially similar manner as seen at FIGS. 58 and 59A-59G. Accordingly, although components of the hypodermic interface assembly 10 (such as, for example, the cannula 12 and the hub 14) are represented at FIGS. 58 and 59A-59G, any of the components (e.g., respectively, the cannula 112, 212, 312, 412 and the hub 114, 214, 314, 414) of the other hypodermic interface assemblies 110, 210, 310, 410 may operate and function in a substantially similar manner as the cannula 12 and the hub 14 and the hypodermic interface assembly 10.

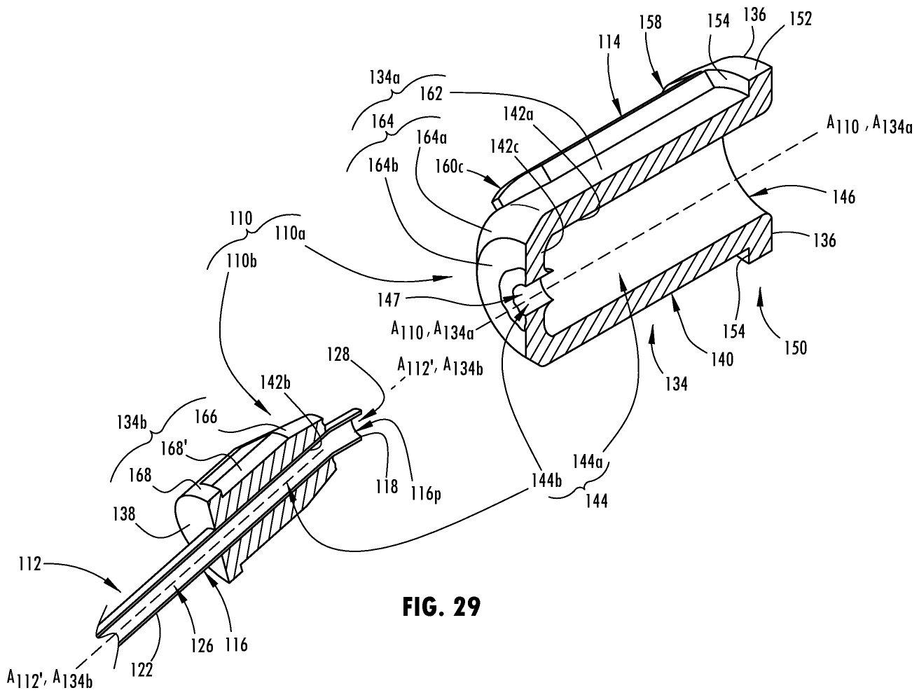

As described above, the design of the hypodermic interface assembly 10 promotes predictable and controlled separation (see, e.g., FIGS. 20A-20B and 59E) of the cannula 12 and the second portion 34b of the substantially tube-shaped body 34 of the hub 14 (that collectively define the second portion 10b of the hypodermic interface assembly 10) relative to the first portion 34a of the substantially tube-shaped body 34 of the hub 14 (that defines the first portion 10a of the hypodermic interface assembly 10). Furthermore, in a substantially similar manner, the design of any of the hypodermic interface assemblies 110, 210, 310, 410 also promotes predictable and controlled separation (see, e.g., respectively FIGS. 29, 38, 47, 56, and 59E) of the cannula 112, 212, 312, 412 and the second portion 134b, 234b, 334b, 434b of the substantially tube-shaped body 134, 234, 334, 434 of the hub 114, 214, 314, 414 (that collectively define the second portion 110b, 210b, 310b, 410b of the hypodermic interface assembly 110, 210, 310, 410) relative to the first portion 134a, 234a, 334a, 434a of the substantially tube-shaped body 134, 234, 334, 434 of the hub 114, 214, 314, 414 (that defines the first portion 110a, 210a, 310a, 410a of the hypodermic interface assembly 110, 210, 310, 410).

In some instances, predictable and controlled separation of the second portion 10b of the hypodermic interface assembly 10 from the first portion 10a of the hypodermic interface assembly 10 may occur after the cannula 12 pierces the subject S (see, e.g., FIGS. 59A-59B). The subject S may be, for example, animalia, such as a human or non-human (i.e., an animal such as, for example, pig or swine). In other examples, the subject S may be an inanimate object. The predicable and controlled separation of the second portion 10b of the hypodermic interface assembly 10 from the first portion 10a of the hypodermic interface assembly 10 mitigates separation of the cannula 12 from the entirety of the hub 14, which may otherwise undesirably result in the cannula 12 being broken-off and subsequently lost (or makes it difficult to easily locate the broken-off cannula) within the flesh of the animalia.

Referring to FIG. 58, the hypodermic interface assembly 10 is shown connected to an injecting device I, such as, for example, an injection gun. The hypodermic interface assembly 10 may be connected to a barrel portion I_{B} of the injection gun I by arranging, for example, the first radially-outward projection or ear 56 and the second radially-outward projection or ear 58 extending from the of the barrel-engaging portion 50 that extends from the outer surface 40 of the substantially tube-shaped body 34 of the hub 14 in corresponding recesses (not shown) formed by the barrel portion I_{B} of the injection gun I and then, for example, quarter-turn locking the hypodermic interface assembly 10 for removably-securing the first radially-outward projection or ear 56 and the second radially-outward projection or ear 58 extending from the of the barrel-engaging portion 50 to the barrel portion I_{B} of the injection gun I.

The injection gun I may include a fluid container C that contains a fluid F (see also, e.g., FIG. 59C). The fluid F may be metered from: (1) the container C; (2) through the injection gun I; (3) into the hypodermic interface assembly 10; and (4) out of the hypodermic interface assembly 10 and into the flesh of the subject S. The injection gun I may be actuated when a user U presses, for example, an actuator IA such as, for example, a trigger in order to cause movement of the fluid F as described above. The injection gun I may be powered in any desirable manner such as, for example: battery powered; air powered; manually powered; or a combination thereof.

Referring to FIG. 59A, the user may grasp the injection gun I and position the sharp piercing tip 32 formed by the distal end surface 20 of the tube-shaped body 16 of the cannula 12 near the outer surface S_{S} of the subject S, which may define the skin or hide of the subject S. Referring to FIGS. 59A-59B, the user U may impart an axial force according to the direction of the arrow X_{A} to the injection gun I along the central axis A₁₀-A₁₀ extending through the hypodermic interface assembly 10 such that the sharp piercing tip 32 formed by the distal end surface 20 of the tube-shaped body 16 of the cannula 12 axially pierces the outer surface S_{S} of the subject S.

Referring to FIGS. 11C and 59C, after the outer surface S_{S} of the subject S has been axially pierced by the cannula 12, the user U may optionally actuate the actuator I_{A} in order to cause movement of the fluid F from: (1) the container C; (2) through the injection gun I; (3) into the hypodermic interface assembly 10; and (4) out of the hypodermic interface assembly 10 and into the flesh of the subject S. In an example, the fluid F may firstly enter the hypodermic interface assembly 10 from the injection gun I at the passage 44 formed by the substantially tube-shaped body 34 of the hub 14 by way of the proximal opening 46 formed by the proximal end surface 36 of the substantially tube-shaped body 34 of the hub 14. Then, the fluid F may secondly enter the passage 26 extending through the tube-shaped body 16 of the cannula 12 by way of the proximal opening 28 formed by the proximal end surface 18 of the body 16 of the cannula 12. Then, thirdly, the fluid F may exit the passage 44 formed by the substantially tube-shaped body 34 of the hub 14 by way of the distal opening 48 formed by the distal end surface 38 of the substantially tube-shaped body 34 of the hub 14. Thereafter, fourthly, the fluid F may exit the passage 26 extending through the tube-shaped body 16 of the cannula 12 by way of the distal opening 30 formed by the distal end surface 20 of the body 16 of the cannula 12.

The fluid F may be any desirable composition that is intended to be delivered to the animalia S. In some instances, the fluid F may be a medicament, a pharmaceutical, a vaccine, an anesthetic, or the like. Accordingly, the fluid F may not include any type of fluid that is not intended to be injected into animalia S. Although the hypodermic interface assembly 10 also may be utilized for injecting fluid F into animalia S, the hypodermic interface assembly 10 may be utilized for removing fluid F (e.g., blood) from animalia S. Therefore, it will be appreciated that the hypodermic interface assembly 10 may deliver or receive fluid F.

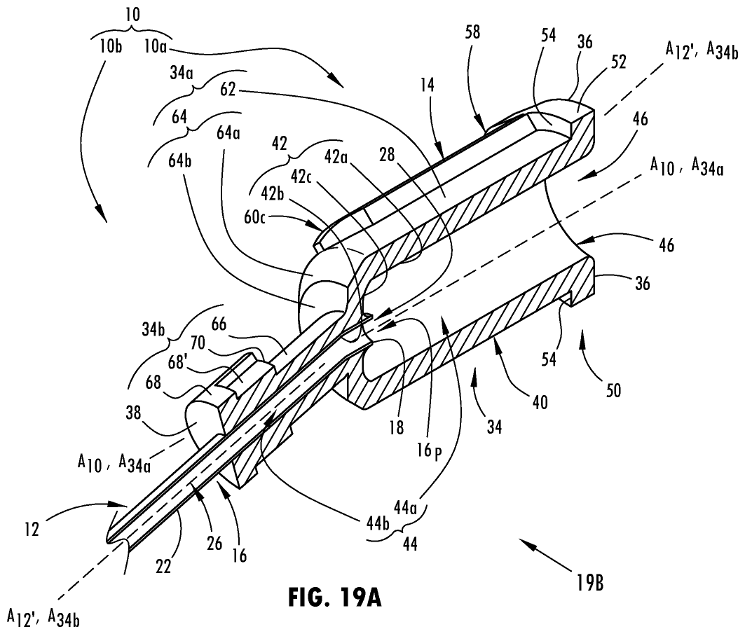

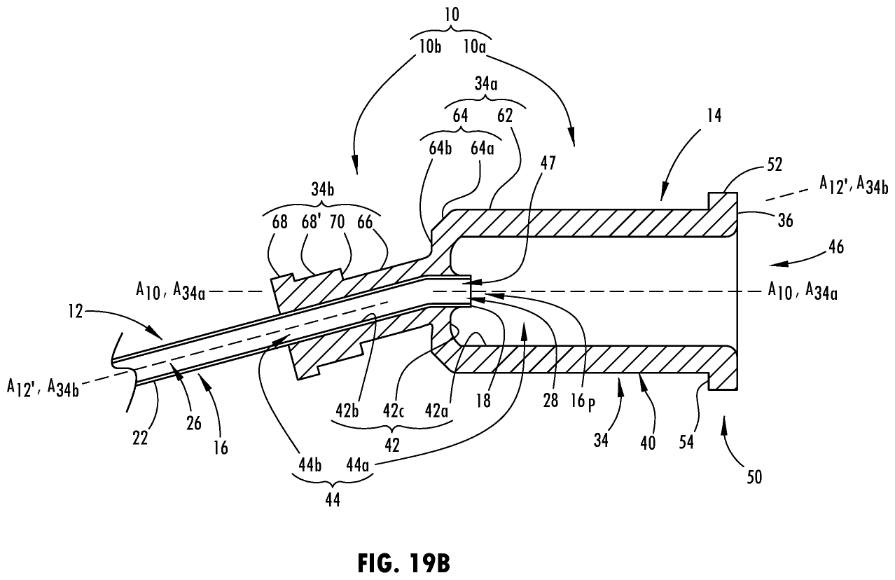

Referring to FIGS. 19A-19B and 59D, after the outer surface S_{S} of the subject S has been axially pierced by the cannula 12, the subject S may experience discomfort as a result of pain arising from the outer surface S_{S} being pierced by the sharp piercing tip 32 formed by the distal end surface 20 of the tube-shaped body 16 of the cannula 12. Accordingly, if the user U is sufficiently grasping the injection gun I, any movement of the subject S may result in the cannula 12 being subjected to one or more radial forces X_{R} relative the central axis A₁₀-A₁₀ extending through the hypodermic interface assembly 10 that may cause the cannula 12 to bend or warp, such that the central axis A₁₂-A₁₂ extending through the axial center of the tube-shaped body 16 of the cannula 12 is not coincident with the central axis A₁₀-A₁₀ extending through the hypodermic interface assembly 10.

Because the hub 14 may be formed from a non-flexible or substantially rigid material (e.g., metal), any stresses imparted to the cannula 12 arising from the one or more radial forces X_{R} is transmitted from the cannula 12 to the hub 14, and, as such, any stresses transmitted from the cannula 12 to the hub 14 are directed to and concentrated at a predetermined portion or region of the hub 14. The predetermined portion or region of the hub 14 that receives the concentrated stresses is generally defined by a portion or region of the hub 14 where the break line B (see, e.g., the break lines B of FIGS. 21A-21G) traverses the hub 14 of the hypodermic interface assembly 10. With respect to the hypodermic interface assemblies 110, 210, 310, 410, the predetermined portion or region of the hub 114, 214, 314, 414 that receives the concentrated stresses is generally defined by a portion or region of the hub 114, 214, 314, 414 where the break line B (see, e.g., respectively, the break lines B of FIGS. 30, 39, 48, 57) traverses the hub 114, 214, 314, 414 of the hypodermic interface assemblies 110, 210, 310, 410.

As described above, the break line B generally demarcates the substantially tube-shaped body 34 of the hub 14 into two portions defined by the first portion 34a of the substantially tube-shaped body 34 of the hub 14 and the second portion 34b of the substantially tube-shaped body 34 of the hub 14. It should be noted that the break line B, which is not an arbitrary region of the hub 14, arises from the design of the hub 14 according to one or a combination of: (1) a selected material that defines the hub 14; (2) the shape or profile of the outer surface 40 of the substantially tube-shaped body 34 of the hub 14; and (3) one or more changes in thicknesses, lengths, widths, dimensions, or shape of the substantially tube-shaped body 34 of the hub 14 relative the central axis A₁₄-A₁₄ of the hub 14 that defines an absence of material defining the substantially tube-shaped body 34 of the hub 14 or a pre-weakened portion of the material defining the substantially tube-shaped body 34 of the hub 14. Accordingly, the exemplary implementations of the hub 14, 114, 214, 314, 414 provided in the present disclosure are not arbitrary design choices and are intentionally configured in order to predictably and controllably separate the second portion 34b of the substantially tube-shaped body 34 of the hub 14 from the first portion 34a of the substantially tube-shaped body 34 of the hub 14, and, as a result, providing for predictable and controlled separation of the second portion 10b of the hypodermic interface assembly 10 from the first portion 10a of the hypodermic interface assembly 10.

As seen at FIG. 59D, as a result of stresses transmitted from the cannula 12 to the hub 14 being directed to and concentrated at a predetermined portion or region of the hub 14, the second portion 34b of the substantially tube-shaped body 34 of the hub 14 may be permitted to also bend or deviate with the cannula 12 away from the central axis A₁₀-A₁₀ extending through the hypodermic interface assembly 10 (see, e.g., the axes A₁₂-A₁₂, A_{34b}-A_{34b} of the cannula 12 and the second portion 34b of the substantially tube-shaped body 34 of the hub 14). Accordingly, the axes A₁₂-A₁₂, A_{34b}-A_{34b} of the cannula 12 and the second portion 34b of the substantially tube-shaped body 34 of the hub 14 generally deviate away from the axis A₃₄ₐ-A₃₄ₐ of the first portion 34a of the substantially tube-shaped body 34 of the hub 14, and axis A₃₄ₐ-A₃₄ₐ may remain coincident with the central axis A₁₀-A₁₀ extending through the hypodermic interface assembly 10.

Referring to FIGS. 20A-20B and 59E (see also, respectively, FIGS. 29, 38, 47, 56 with respect to the hypodermic interface assemblies 110, 210, 310, 410), the stresses transmitted from the cannula 12 to the hub 14 that were directed to and concentrated at the predetermined portion or region of the hub 14 continues to bend the second portion 34b of the substantially tube-shaped body 34 of the hub 14 relative the first portion 34a of the substantially tube-shaped body 34 of the hub 14 until the structural integrity of the material defining the hub 14 fails. As a result, the second portion 34b of the substantially tube-shaped body 34 of the hub 14 predictably and controllably separates from the first portion 34a of the substantially tube-shaped body 34 of the hub 14 (and, as a result, the second portion 10b of the hypodermic interface assembly 10 predictably and controllably separates from the first portion 10a of the hypodermic interface assembly 10 at or substantially at, about, along, or on break line B).

With reference to FIGS. 21A-21C and 59D-59E, because the proximal end 16_{P} of the tube-shaped body 16 of the cannula 12 is arranged within a portion of the passage 44 defined by first portion 34a of the substantially tube-shaped body 34 of the hub 14 (i.e., the proximal end 16_{P} of the tube-shaped body 16 of the cannula 12 is arranged upstream of the break line B), the proximal end 16_{P} of the tube-shaped body 16 of the cannula 12 is configured to be “ripped” out of the first portion 10a of the hypodermic interface assembly 10 when the second portion 34b (that is joined to the cannula 12) of the substantially tube-shaped body 34 of the hub 14 predictably and controllably separates from the first portion 34a of the substantially tube-shaped body 34 of the hub 14. In some instances, the proximal end 16_{P} of the tube-shaped body 16 of the cannula 12 that is configured to be “ripped” out of the first portion 10a of the hypodermic interface assembly 10 may be deformed, bent, or warped.

Alternatively, with respect to the exemplary implementations of the hypodermic interface assembly 10 represented at FIG. 21D, the proximal end surface 18 of the body 16 of the cannula 12 may be substantially aligned with the break line B, and, as such, the proximal end 16_{P} of the tube-shaped body 16 of the cannula 12 is not arranged within the portion of the passage 44 defined by first portion 34a of the substantially tube-shaped body 34 of the hub 14. Accordingly, the proximal end 16_{P} of the tube-shaped body 16 of the cannula 12 may not be configured to be “ripped” out of the first portion 10a of the hypodermic interface assembly 10 as described above according to the exemplary implementations of the hypodermic interface assembly 10 at FIGS. 21A-21C. Rather, the proximal end 16_{P} of the tube-shaped body 16 of the cannula 12 may be configured to be “migrate away from” the first portion 10a of the hypodermic interface assembly 10 and may not be deformed, bent, or warped when the second portion 10b of the hypodermic interface assembly 10 predictably and controllably separates from the first portion 10a of the hypodermic interface assembly 10.

Furthermore, with respect to the exemplary implementations of the hypodermic interface assembly 10 represented at FIGS. 21E-21Q the proximal end surface 18 of the body 16 of the cannula 12 may be arranged downstream of the break line B, and, as such, the proximal end 16_{P} of the tube-shaped body 16 of the cannula 12 is not arranged within the portion of the passage 44 defined by first portion 34a of the substantially tube-shaped body 34 of the hub 14. Accordingly, the proximal end 16_{P} of the tube-shaped body 16 of the cannula 12 may not be configured to be “ripped” out of the first portion 10a of the hypodermic interface assembly 10 as described above according to the exemplary implementations of the hypodermic interface assembly 10 at FIGS. 21A-21C. Rather, the proximal end 16_{P} of the tube-shaped body 16 of the cannula 12 may be configured to be “migrate away from” the first portion 10a of the hypodermic interface assembly 10 and may not be deformed, bent, or warped when the second portion 10b of the hypodermic interface assembly 10 predictably and controllably separates from the first portion 10a of the hypodermic interface assembly 10.

Irrespective of which exemplary arrangement of the cannula 12 relative to the hub 14 (as described at FIGS. 21A-21G) is utilized, the cannula 12 does not break when the second portion 10b of the hypodermic interface assembly 10 predictably and controllably separates from the first portion 10a of the hypodermic interface assembly 10. That is, the only component of the hypodermic interface assembly 10 that “breaks” is the hub 14 when the second portion 34b (that is non-separably joined to the cannula 12) of the substantially tube-shaped body 34 of the hub 14 predictably and controllably separates from the first portion 34a of the substantially tube-shaped body 34 of the hub 14). In other words, in some instances, the design of the hypodermic interface assembly 10 contributes to the cannula 12 remaining intact (i.e., not broken) when the second portion 10b of the hypodermic interface assembly 10 predictably and controllably separates from the first portion 10a of the hypodermic interface assembly 10. To the extent that any portion of the cannula 12 is structurally compromised, such structural compromise may potentially occur when the cannula is “ripped” out of the first portion 10a of the hypodermic interface assembly 10 as described above according to the exemplary implementations of the hypodermic interface assembly 10 at FIGS. 21A-21C.

As seen at FIG. 59E, because the second portion 34b of the substantially tube-shaped body 34 of the hub 14 is non-separably joined to the cannula 12, the user U, may easily identify a location of the animalia S where the cannula 12 is impaled within the flesh of the animalia S. The location of the animalia S where the cannula 12 is impaled within the flesh of the animalia S is easily identifiable as a result of, for example, the second portion 34b of the substantially tube-shaped body 34 of the hub 14 of the second component of the second portion 10b of the hypodermic interface assembly 10 resting upon the skin S_{S} or hide of the animalia S (while the cannula 12 is not visible to the user U since the cannula 12 is contained within and obscured by the flesh of the animalia S.

Thereafter, as seen at FIG. 59F, the user U may pinch or grasp the second portion 10b of the hypodermic interface assembly 10 and apply a pulling force to the second portion 10b of the hypodermic interface assembly 10 (that also includes the impaled cannula 12). As seen at FIG. 59Q as a result of the pulling force to the second portion 10b of the hypodermic interface assembly 10 by the user U, the cannula 12 is removed from the flesh of the animalia S such that the cannula 12 otherwise is not lost or would undesirably remain within the flesh of the animalia S.

A number of implementations have been described. Nevertheless, it will be understood that various modifications may be made without departing from the spirit and scope of the disclosure. Accordingly, other implementations are within the scope of the following claims. For example, the actions recited in the claims can be performed in a different order and still achieve desirable results.

The terminology used herein is for the purpose of describing particular exemplary configurations only and is not intended to be limiting. As used herein, the singular articles “a,” “an,” and “the” may be intended to include the plural forms as well, unless the context clearly indicates otherwise. The terms “comprises,” “comprising,” “including,” and “having,” are inclusive and therefore specify the presence of features, steps, operations, elements, and/or components, but do not preclude the presence or addition of one or more other features, steps, operations, elements, components, and/or groups thereof. The method steps, processes, and operations described herein are not to be construed as necessarily requiring their performance in the particular order discussed or illustrated, unless specifically identified as an order of performance. Additional or alternative steps may be employed.

When an element or layer is referred to as being “on,” “engaged to,” “connected to,” “attached to,” or “coupled to” another element or layer, it may be directly on, engaged, connected, attached, or coupled to the other element or layer, or intervening elements or layers may be present. In contrast, when an element is referred to as being “directly on,” “directly engaged to,” “directly connected to,” “directly attached to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The terms first, second, third, etc. may be used herein to describe various elements, components, regions, layers and/or sections. These elements, components, regions, layers and/or sections should not be limited by these terms. These terms may be only used to distinguish one element, component, region, layer or section from another region, layer or section. Terms such as “first,” “second,” and other numerical terms do not imply a sequence or order unless clearly indicated by the context. Thus, a first element, component, region, layer or section discussed below could be termed a second element, component, region, layer or section without departing from the teachings of the example configurations.

What is claimed is:

1. A hypodermic interface assembly comprising:
a cannula; and
a hub defined by a first portion and a second portion,
wherein the second portion of the hub is breakably-connected to the first portion of the hub,
wherein the cannula is joined to the second portion of the hub,
wherein the second portion of the hub is defined by an outer head surface portion having a first geometry,
wherein the second portion of the hub is defined by an outer neck surface portion having a second geometry,
wherein the first geometry of the outer head surface portion is greater than the second geometry of the outer neck or groove surface portion.

2. The hypodermic interface assembly of claim 1, wherein the outer neck surface portion defines a substantially cylindrical neck portion.

3. The hypodermic interface assembly of claim 1, wherein the outer neck surface portion defines a substantially conical neck portion.

4. The hypodermic interface assembly of claim 1, wherein the outer neck surface portion defines a first surface portion that contributes to forming a V-shaped groove.

5. The hypodermic interface assembly of claim 1, wherein the cannula is disposed within a hub passage extending through the first portion and the second portion of the hub, wherein the hub passage includes:
a first hub passage portion extending through the first portion of the hub; and
a second hub passage portion extending through the second portion of the hub, wherein an outer side surface of the cannula is secured to an inner surface portion of an inner surface of the hub that defines the hub passage, wherein the inner surface portion that is defined by the inner surface of the hub extends through the second portion of the hub.

6. The hypodermic interface assembly of claim 5, wherein a proximal of the cannula is arranged within and extends into the first hub passage portion extending through the first portion of the hub.

7. The hypodermic interface assembly of claim 5, wherein all of the second hub passage portion extending through the second portion of the hub contains a portion of a length of the cannula that extends from a proximal end of the cannula.

8. The hypodermic interface assembly of claim 5, wherein a portion of the second hub passage portion extending through the second portion of the hub contains a portion of a length of the cannula that extends from a proximal end of the cannula.

9. A hypodermic interface assembly comprising:
a first hypodermic interface assembly portion that is defined by:
a first portion of a hub body of a hub; and
a second hypodermic interface assembly portion that is frangibly-connected to and separable from the first hypodermic interface assembly portion, wherein the second hypodermic interface assembly portion is defined by:
a cannula; and
a second portion of the hub body of the hub, wherein the cannula is joined to the second portion of the hub body of the hub.

10. The hypodermic interface assembly of claim 9,
wherein the second portion of the hub body of the hub is defined by an outer head surface portion having a first geometry and an outer neck surface portion having a second geometry.

11. The hypodermic interface assembly of claim 10, wherein the first geometry of the outer head surface portion is greater than the second geometry of the outer neck or groove surface portion.

12. The hypodermic interface assembly of claim 10, wherein the outer neck surface portion defines a substantially cylindrical neck portion.

13. The hypodermic interface assembly of claim 10, wherein the outer neck surface portion defines a substantially conical neck portion.

14. The hypodermic interface assembly of claim 10, wherein the outer neck surface portion defines a first surface portion that contributes to forming a V-shaped groove.

15. The hypodermic interface assembly of claim 10, wherein at least a portion of a thickness of a neck of the hub extending along at least a portion of a length of the outer neck surface portion is defined by a ratio ranging between approximately 0.10 and 0.95 times a thickness of a head of the hub defined by the outer head surface.

16. The hypodermic interface assembly of claim 9, wherein the cannula is disposed within a hub passage extending through the hub body of the hub, wherein the hub passage includes:
a first hub passage portion extending through the first portion of the hub body of the hub; and
a second hub passage portion extending through the second portion of the hub body of the hub, wherein an outer side surface of the cannula is secured to an inner surface portion of an inner surface of the hub body of the hub that defines the hub passage, wherein the inner surface portion that is defined by the inner surface of the hub body of the hub extends through the second portion of the hub body of the hub.

17. The hypodermic interface assembly of claim 16, wherein a proximal end of the cannula is arranged within and extends into the first hub passage portion extending through the first portion of the hub body of the hub.

18. The hypodermic interface assembly of claim 16, wherein all of the second hub passage portion extending through the second portion of the hub body of the hub contains a portion of a length of the cannula that extends from a proximal end of the cannula.

19. The hypodermic interface assembly of claim 16, wherein a portion of the second hub passage portion extending through the second portion of the hub body of the hub contains a portion of a length of the cannula that extends from a proximal end of the cannula.

20. A hypodermic interface assembly comprising:
a cannula;
a hub defined by a first portion and a second portion,
wherein the second portion of the hub is breakably-connected to the first portion of the hub,
wherein the cannula is joined to the second portion of the hub
wherein the second portion of the hub is defined by an outer head surface portion having a first geometry,
wherein the second portion of the hub is separated from the a first portion of the hub by a groove surface portion having a second geometry that is less than a portion of the first geometry of the outer head surface portion.

21. The hypodermic interface assembly of claim 20, wherein at least a portion of a thickness of a neck of the hub extending along at least a portion of a length of the hub is defined by a ratio ranging between approximately 0.10 and 1.50 times a thickness of a head of the hub defined by the outer head surface portion.

22. A method comprising:
providing a hub that is defined by a first portion and a second portion, wherein the second portion of the hub is breakably-connected to the first portion of the hub; and
non-separably joining a cannula to the second portion of the hub.

23. The method of claim 22 further comprising:
separably joining the first portion of the hub to an injection gun; and
inserting the cannula into the flesh of a subject.

24. The method of claim 23 further comprising:
subjecting one or both of the cannula and the hub to one or more radial forces relative to a central axis extending through the cannula and the hub for mechanically-separating the first portion of the hub and the second portion of the hub,
whereby the first portion of the hub remains separably joined to the injection gun, the cannula is removably disposed within the flesh of the subject, and the second portion of the hub is disposed adjacent an outer surface of the flesh of the subject.

25. The method of claim 24 further comprising:
locating the second portion of the hub that is disposed adjacent the outer surface of the flesh of the subject;
grasping the second portion of the hub that is disposed adjacent the outer surface of the flesh of the subject; and
applying a force to the second portion of the hub to remove the cannula from the flesh of the subject.

26. The method of claim 24 further comprising:
separating the first portion of the hub from the injection gun.
